SEQUENCE LISTING (21 sequences)

SEQ ID NO: 1 - DNA, 435 bp, synthetic construct
  source 1..435: mol_type = other DNA
atgttggtattgttgccagacgaagtttcaggtttagagcagttggaatccatcataaactttgagaagttgacagaatggacaagttctaacgtcatggaagaaaggaagataaaggtctacttaccaagaatgaagatggaagagaagtataacttgacttcagtattgatggcaatgggcataactgatgtctttagttcatctgccaatttgtctggaattagttctgctgaatccttgaagatttctcaagctgttcatgctgctcatgcagagattaacgaagctggtcgtgaggtagttggatcagcagaagctggagtagatgcagcttccgtatccgaagagtttcgtgccgaccatcccttcttattctgtatcaagcatatcgccactaatgccgtattgttctttggtaggtgcgtctcacct

SEQ ID NO: 2 - DNA, 195 bp, synthetic construct
  source 1..195: mol_type = other DNA
atggcaataccaccaaagaagaaccaagataagacagaaattccaacaatcaacaccattgcaagtggcgaaccaacatcaactcctactaccgaagcagtcgaatcaacagttgcaactttggaagattctccagaggttattgagtcaccacccgaaatcaatactgtgcaagttacctcaactgctgtctaa

SEQ ID NO: 3 - DNA, 426 bp, synthetic construct
  source 1..426: mol_type = other DNA
atgatgtcctttgtcagtttgttgttagtaggaatcttgttccatgcaactcaagctgaacaattgacaaagtgcgaagtattcagagagttgaaggacttgaaaggttacggtggtgtaagtttaccagaatgggtgtgtactgcctttcacacttctggttatgacacacaggctatagtccagaataacgattccaccgaatatggtttgtttcagatcaacaataagatttggtgtaaagatgaccagaaccctcactcatccaatatctgtaacataagttgcgacaagttcttagatgacgatttgacagatgacattatgtgcgtcaagaagatattggataaagtcggtatcaactattggttagcacacaaagcattatgttcagagaaattggatcagtggttatgcgagaagttg

SEQ ID NO: 4 - DNA, 669 bp, synthetic construct
  source 1..669: mol_type = other DNA
atgaaattcttcatattcacctgcttattggcagtggccttagctaaacacaagatggaacatgtatcctcctccgaagaaccaatcaatatctcccaagaaatctataagcaagagaagaatatggctattcatccaagaaaggagaagttatgcactacaagttgtgaagaagtagttagaaatgctgacgaagaagaatactctataagaagttcttctgaggaaagtgcagaggttgcaccagaagaggtgaagattactgttgatgacaagcactatcagaaggctttgaacgaaatcaaccagttctaccagaagttccctcaatacttacaatacttataccaaggaccaatcgtcttgaacccttgggaccaagtcaagagaaatgcaggtcctttcactccaaccgtgaacagagaacaattgtctacatccgaagagaatagtaagaagacaattgacatggaaagtacagaagtctttactaagaagactaagttgactgaagaggagaagaatcgtttgaacttcttgaagaagatcagtcaatactaccagaagtttgcttggcctcaatacttaaagacagtggatcaacatcagaaggctatgaagccctggacacaacctaagactaacgctattccttatgtcagatactta

SEQ ID NO: 5 - DNA, 9634 bp, synthetic construct
  source 1..9634: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatgaaattcttcatattcacctgcttattggcagtggccttagctaaacacaagatggaacatgtatcctcctccgaagaaccaatcaatatctcccaagaaatctataagcaagagaagaatatggctattcatccaagaaaggagaagttatgcactacaagttgtgaagaagtagttagaaatgctgacgaagaagaatactctataagaagttcttctgaggaaagtgcagaggttgcaccagaagaggtgaagattactgttgatgacaagcactatcagaaggctttgaacgaaatcaaccagttctaccagaagttccctcaatacttacaatacttataccaaggaccaatcgtcttgaacccttgggaccaagtcaagagaaatgcaggtcctttcactccaaccgtgaacagagaacaattgtctacatccgaagagaatagtaagaagacaattgacatggaaagtacagaagtctttactaagaagactaagttgactgaagaggagaagaatcgtttgaacttcttgaagaagatcagtcaatactaccagaagtttgcttggcctcaatacttaaagacagtggatcaacatcagaaggctatgaagccctggacacaacctaagactaacgctattccttatgtcagatacttataaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgttggtattgttgccagacgaagtttcaggtttagagcagttggaatccatcataaactttgagaagttgacagaatggacaagttctaacgtcatggaagaaaggaagataaaggtctacttaccaagaatgaagatggaagagaagtataacttgacttcagtattgatggcaatgggcataactgatgtctttagttcatctgccaatttgtctggaattagttctgctgaatccttgaagatttctcaagctgttcatgctgctcatgcagagattaacgaagctggtcgtgaggtagttggatcagcagaagctggagtagatgcagcttccgtatccgaagagtttcgtgccgaccatcccttcttattctgtatcaagcatatcgccactaatgccgtattgttctttggtaggtgcgtctcaccttaagcatgctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcgcgccaccatgatgtcctttgtcagtttgttgttagtaggaatcttgttccatgcaactcaagctgaacaattgacaaagtgcgaagtattcagagagttgaaggacttgaaaggttacggtggtgtaagtttaccagaatgggtgtgtactgcctttcacacttctggttatgacacacaggctatagtccagaataacgattccaccgaatatggtttgtttcagatcaacaataagatttggtgtaaagatgaccagaaccctcactcatccaatatctgtaacataagttgcgacaagttcttagatgacgatttgacagatgacattatgtgcgtcaagaagatattggataaagtcggtatcaactattggttagcacacaaagcattatgttcagagaaattggatcagtggttatgcgagaagttgtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggcaataccaccaaagaagaaccaagataagacagaaattccaacaatcaacaccattgcaagtggcgaaccaacatcaactcctactaccgaagcagtcgaatcaacagttgcaactttggaagattctccagaggttattgagtcaccacccgaaatcaatactgtgcaagttacctcaactgctgtctaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 6 - DNA, 1606 bp, synthetic construct
  source 1..1606: mol_type = other DNA
atgggtttgagtggtgccactatctcagcaccattaggctgctgtgttttaagatgcggcgctgtgggtggtggaaaagcattgaaagctgatgctgaaaggtggagaagagctggttggtccagaagggtaggtggtccaaaagtgagatgcgtggcaaccgagaaacatgatgaaactgcagcagtgggtgcagctgttggcgtcgattttgctgatgaggaagactatagaaaaggaggtggaggagaattgttgtatgttcagatgcaaagtacaaagcctatggagagtcaatctaagatagcttctaagttgtctccaatcagtgatgaaaacactgtcttggacttagttattataggatgtggcccagcaggtttatcattggcttctgaatcagcaaagaaaggtttgactgttggcttaatcggtcctgacttgccttttacaaataactatggtgtttgggaggatgaattcaaagatttgggtttggaatcctgtattgaacacgtatggaaagatactatagtttatttggataataataagcctattttgattggaagatcttatggcagagtacacagagatttattacacgaggaattattaaagagatgttacgaagccggtgtcacatacttaaactctaaagtggataagataattgaaagtccagacggccatagagtagtttgttgtgataaaggtagggaaatcatatgtaggttggcaatcgtcgcttcaggagccgccagtggtaggttattggaatacgaggtcggcggccccagagtttgtgtccagacagcttatggagtagaggtagaagtcgagaacaatccctatgacccatccttaatggtctttatggattacagagattgcttcaaagaagaattttctcatacagaacaagaaaaccccactttcttgtatgcaatgcctatgtcaccaaccagagtgttctttgaagagacctgcttagcatctaaagatgcaatgtcatttgatttattaaagaaaagattgatgtacagattgaatgctatgggaataaggatattgaaggtgtatgaagaagaatggagttacatcccagttggtggttcattgcctaacacagaccaaaagaatttagcttttggtgctgctgcaagtatggttcatcctgccaccggatattccgttgttaggtccttgtccgaagcacctaggtacgcttcagtaatttcagacattttaggtaatagagttcccgctgaatacatgttgggaaactcccaaaattactcaccatcaatgttagcctggaggaccttgtggccacaagagagaaagaggcagaggtctttcttcttattcggattagccttgattattcaattaaacaatgagggtattcaaactttcttcgaggccttctttagagtaccaagatggatgtggagaggtttcttaggttctacattgtcttctgttgacttgatcttattttctttctatatgttcgccattgccccaaatcagttgagaatgaatttagttagacatttgttatctgaccctacaggttcctctatgattaagacttatttaa

SEQ ID NO: 7 - DNA, 1024 bp, synthetic construct
  source 1..1024: mol_type = other DNA
atgacccagagttctgctttacatcagcaaccaagaccagtatccacaggctataggtctgttcccagagagttcgttgatcctcctcctgcatggaatcccaccgttgctttgttcttgggaggatatggcttagccgcttttactatttggggttggttcttgggtggtttgcccttaccagtattattgtgcactggattcttggctttacatttggaaggtactgtgattcatgacgcatgtcataacgctgctcatcctaataggtggttaaatcaagccatgggtcatggaagtgctttgttgttaggattctctttccccgttttcactagagtgcacttggagcatcatgctcacgtcaatgaccccaagaacgacccagatcatattgtttcaacttttggcccattgtggttaattgcccctagattcttttatcacgaatggttcttctttcagaggagattatggaggaggtgggaattgatgcaatggggattggagagatccgtatttgtggtcattgtattatctgcagcaagatttgagttcttgccattcattttcaactgttggtttgctcctgcattgatggtcggtgtgactttaggtttgttctttgattacttgccacataggccatttacatcaagaaatagatggacaaatgctagaatatatcctggtaggttgatgaactggttgataatgggccaaaattaccacttagttcaccatttatggccatcaataccatggtttgaatacaaacctgcatatgaagccacaaagccattgttagattctaaaggtagtccacaaagattaggtatattcgagacaaggagagatggctataacttcttatacgatatattagttggtgttagatcacacaagagaagaaggggtaaaatgagaagggccgcaaggttcatgccaatgagatccttccaaagacactggttaggtttcgtcgatagaatcgccatcaaaaccgaacctagaagacctttaa

SEQ ID NO: 8 - DNA, 2160 bp, synthetic construct
  source 1..2160: mol_type = other DNA
gccaccatggcggttgcaggatcgaccatgaacctgcatctcacttcatctccatacaagacagttccatcactctgtaaattcaccagaaaacagttccgattaaaggcctctgcaacgaatccagacgctgaagatgggaagatgatgtttaaaaacgataaacccaatttgaaggtcgaattcgctggggagaaaccggtgacaccattactggataccattaattaccctgtgcacatgaaaaacctcaccactcaggatcttgagcaattagcagcagaacttagacaagatattgtatattcagtagcgaatacaggtggtcatttgagttcaagtttaggtgttgttgaattgtctgttgctttacaccatgttttcaacaccccagatgacaagatcatttgggacgttggtcaccaggcatacccacataagattttgaccggaagaaggtcaaagatgcacaccataagaaaaacttctggtttagctggttttcctaaacgagatgaaagtgctcatgatgcttttggtgctggacatagttctacaagcatctctgctggactaggtatggctgtcggtagagatttattagggaaaaccaacaacgtgatatcggtgatcggagatggcgccatgacggccggacgagcatatgaggcgataaataatgcaggatttcttgattcaaatctaatcgtcgttttaaacgacaacaagcaagtttcattaccgactgccacgttggacggacctgcaactcccgtcggggctctcagcggcgctttatccaaattgcaagccagtaccaagttccgtaagcttcgtgaagccgccaagagcattactaaacaaattggacctcaagcacatgaagtggcggcgaaagtcgacgaatacgcaagaggtatgattagtgctagcgggtcgactttattcgaggaactcggattatactacatcggtcccgtcgatggtcacaatgttgaagatttagtcaacatttttgaaaaagtcaagtcaatgcccgcacccggaccggttctaatccacatcgtgaccgaaaaaggcaaaggttaccctcctgctgaagccgctgctgaccgtatgcacggagttgtgaagtttgatgttccaactggaaaacaattcaagacaaaatcaccgacactttcgtatactcagtattttgctgaatcacttataaaagaagctgaagctgataacaagattgtcgcgatacacgccgccatgggaggcggtaccggactcaattacttccagaagaagtgtcctgaacgttgttttgatgtcggtatcgcggaacaacacgcagttactttcgccgcgggtttagccaccgaaggtcttaaaccattttgcgcgatctattcgtcgtttttgcaacgaggatacgatcaagtggtgcatgacgttgatctacaaaagttaccggttcggtttgcgatggaccgagctggtttagtcggggctgatggaccgacacattgtggtgcgtttgacataacctacatggcgtgtctaccaaacatggtggtgatggctccagccgatgaagccgaattgatgcacatggttgcaacggctgcagccattgacgacagaccgagttgctttcggttcccaagaggcaatggcattggtgcaccacttcctcctaataacaaagggattcccatagaggttggtaaaggaagaatattacttgaaggaactcgtgttgcgatattgggatacggttcgatagttcaagaatgtctaggtgcggctagcttgcttcaagcccataacgtgtctgcaaccgtagccgatgcgcggttctgcaaaccgttagacaccggactgattagacgattagccaacgagcatgaagtcttacttaccgtagaggaaggctcgattggtggatttggatcacacgttgctcactttctaagcttaaatggtctcttagatggaaaacttaagcttagagcaatgactcttcctgataaatacattgatcatggtgcaccacaagatcagcttgaagaagccggtctttcttcaaaacatatttgttcatctcttttatcacttttgggaaaacctaaagaagcacttcaatacaaatcaataatgtaatctaga

SEQ ID NO: 9 - DNA, 10563 bp, synthetic construct
  source 1..10563: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatgggtttgagtggtgccactatctcagcaccattaggctgctgtgttttaagatgcggcgctgtgggtggtggaaaagcattgaaagctgatgctgaaaggtggagaagagctggttggtccagaagggtaggtggtccaaaagtgagatgcgtggcaaccgagaaacatgatgaaactgcagcagtgggtgcagctgttggcgtcgattttgctgatgaggaagactatagaaaaggaggtggaggagaattgttgtatgttcagatgcaaagtacaaagcctatggagagtcaatctaagatagcttctaagttgtctccaatcagtgatgaaaacactgtcttggacttagttattataggatgtggcccagcaggtttatcattggcttctgaatcagcaaagaaaggtttgactgttggcttaatcggtcctgacttgccttttacaaataactatggtgtttgggaggatgaattcaaagatttgggtttggaatcctgtattgaacacgtatggaaagatactatagtttatttggataataataagcctattttgattggaagatcttatggcagagtacacagagatttattacacgaggaattattaaagagatgttacgaagccggtgtcacatacttaaactctaaagtggataagataattgaaagtccagacggccatagagtagtttgttgtgataaaggtagggaaatcatatgtaggttggcaatcgtcgcttcaggagccgccagtggtaggttattggaatacgaggtcggcggccccagagtttgtgtccagacagcttatggagtagaggtagaagtcgagaacaatccctatgacccatccttaatggtctttatggattacagagattgcttcaaagaagaattttctcatacagaacaagaaaaccccactttcttgtatgcaatgcctatgtcaccaaccagagtgttctttgaagagacctgcttagcatctaaagatgcaatgtcatttgatttattaaagaaaagattgatgtacagattgaatgctatgggaataaggatattgaaggtgtatgaagaagaatggagttacatcccagttggtggttcattgcctaacacagaccaaaagaatttagcttttggtgctgctgcaagtatggttcatcctgccaccggatattccgttgttaggtccttgtccgaagcacctaggtacgcttcagtaatttcagacattttaggtaatagagttcccgctgaatacatgttgggaaactcccaaaattactcaccatcaatgttagcctggaggaccttgtggccacaagagagaaagaggcagaggtctttcttcttattcggattagccttgattattcaattaaacaatgagggtattcaaactttcttcgaggccttctttagagtaccaagatggatgtggagaggtttcttaggttctacattgtcttctgttgacttgatcttattttctttctatatgttcgccattgccccaaatcagttgagaatgaatttagttagacatttgttatctgaccctacaggttcctctatgattaagacttatttaactttataatgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgacccagagttctgctttacatcagcaaccaagaccagtatccacaggctataggtctgttcccagagagttcgttgatcctcctcctgcatggaatcccaccgttgctttgttcttgggaggatatggcttagccgcttttactatttggggttggttcttgggtggtttgcccttaccagtattattgtgcactggattcttggctttacatttggaaggtactgtgattcatgacgcatgtcataacgctgctcatcctaataggtggttaaatcaagccatgggtcatggaagtgctttgttgttaggattctctttccccgttttcactagagtgcacttggagcatcatgctcacgtcaatgaccccaagaacgacccagatcatattgtttcaacttttggcccattgtggttaattgcccctagattcttttatcacgaatggttcttctttcagaggagattatggaggaggtgggaattgatgcaatggggattggagagatccgtatttgtggtcattgtattatctgcagcaagatttgagttcttgccattcattttcaactgttggtttgctcctgcattgatggtcggtgtgactttaggtttgttctttgattacttgccacataggccatttacatcaagaaatagatggacaaatgctagaatatatcctggtaggttgatgaactggttgataatgggccaaaattaccacttagttcaccatttatggccatcaataccatggtttgaatacaaacctgcatatgaagccacaaagccattgttagattctaaaggtagtccacaaagattaggtatattcgagacaaggagagatggctataacttcttatacgatatattagttggtgttagatcacacaagagaagaaggggtaaaatgagaagggccgcaaggttcatgccaatgagatccttccaaagacactggttaggtttcgtcgatagaatcgccatcaaaaccgaacctagaagacctttaaagagataaggatccgaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 10 - DNA, 13407 bp, synthetic construct
  source 1..13407: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctaggatgggtttgagtggtgccactatctcagcaccattaggctgctgtgttttaagatgcggcgctgtgggtggtggaaaagcattgaaagctgatgctgaaaggtggagaagagctggttggtccagaagggtaggtggtccaaaagtgagatgcgtggcaaccgagaaacatgatgaaactgcagcagtgggtgcagctgttggcgtcgattttgctgatgaggaagactatagaaaaggaggtggaggagaattgttgtatgttcagatgcaaagtacaaagcctatggagagtcaatctaagatagcttctaagttgtctccaatcagtgatgaaaacactgtcttggacttagttattataggatgtggcccagcaggtttatcattggcttctgaatcagcaaagaaaggtttgactgttggcttaatcggtcctgacttgccttttacaaataactatggtgtttgggaggatgaattcaaagatttgggtttggaatcctgtattgaacacgtatggaaagatactatagtttatttggataataataagcctattttgattggaagatcttatggcagagtacacagagatttattacacgaggaattattaaagagatgttacgaagccggtgtcacatacttaaactctaaagtggataagataattgaaagtccagacggccatagagtagtttgttgtgataaaggtagggaaatcatatgtaggttggcaatcgtcgcttcaggagccgccagtggtaggttattggaatacgaggtcggcggccccagagtttgtgtccagacagcttatggagtagaggtagaagtcgagaacaatccctatgacccatccttaatggtctttatggattacagagattgcttcaaagaagaattttctcatacagaacaagaaaaccccactttcttgtatgcaatgcctatgtcaccaaccagagtgttctttgaagagacctgcttagcatctaaagatgcaatgtcatttgatttattaaagaaaagattgatgtacagattgaatgctatgggaataaggatattgaaggtgtatgaagaagaatggagttacatcccagttggtggttcattgcctaacacagaccaaaagaatttagcttttggtgctgctgcaagtatggttcatcctgccaccggatattccgttgttaggtccttgtccgaagcacctaggtacgcttcagtaatttcagacattttaggtaatagagttcccgctgaatacatgttgggaaactcccaaaattactcaccatcaatgttagcctggaggaccttgtggccacaagagagaaagaggcagaggtctttcttcttattcggattagccttgattattcaattaaacaatgagggtattcaaactttcttcgaggccttctttagagtaccaagatggatgtggagaggtttcttaggttctacattgtcttctgttgacttgatcttattttctttctatatgttcgccattgccccaaatcagttgagaatgaatttagttagacatttgttatctgaccctacaggttcctctatgattaagacttatttaactttataatgattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgacccagagttctgctttacatcagcaaccaagaccagtatccacaggctataggtctgttcccagagagttcgttgatcctcctcctgcatggaatcccaccgttgctttgttcttgggaggatatggcttagccgcttttactatttggggttggttcttgggtggtttgcccttaccagtattattgtgcactggattcttggctttacatttggaaggtactgtgattcatgacgcatgtcataacgctgctcatcctaataggtggttaaatcaagccatgggtcatggaagtgctttgttgttaggattctctttccccgttttcactagagtgcacttggagcatcatgctcacgtcaatgaccccaagaacgacccagatcatattgtttcaacttttggcccattgtggttaattgcccctagattcttttatcacgaatggttcttctttcagaggagattatggaggaggtgggaattgatgcaatggggattggagagatccgtatttgtggtcattgtattatctgcagcaagatttgagttcttgccattcattttcaactgttggtttgctcctgcattgatggtcggtgtgactttaggtttgttctttgattacttgccacataggccatttacatcaagaaatagatggacaaatgctagaatatatcctggtaggttgatgaactggttgataatgggccaaaattaccacttagttcaccatttatggccatcaataccatggtttgaatacaaacctgcatatgaagccacaaagccattgttagattctaaaggtagtccacaaagattaggtatattcgagacaaggagagatggctataacttcttatacgatatattagttggtgttagatcacacaagagaagaaggggtaaaatgagaagggccgcaaggttcatgccaatgagatccttccaaagacactggttaggtttcgtcgatagaatcgccatcaaaaccgaacctagaagacctttaaagagataaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccatggcggttgcaggatcgaccatgaacctgcatctcacttcatctccatacaagacagttccatcactctgtaaattcaccagaaaacagttccgattaaaggcctctgcaacgaatccagacgctgaagatgggaagatgatgtttaaaaacgataaacccaatttgaaggtcgaattcgctggggagaaaccggtgacaccattactggataccattaattaccctgtgcacatgaaaaacctcaccactcaggatcttgagcaattagcagcagaacttagacaagatattgtatattcagtagcgaatacaggtggtcatttgagttcaagtttaggtgttgttgaattgtctgttgctttacaccatgttttcaacaccccagatgacaagatcatttgggacgttggtcaccaggcatacccacataagattttgaccggaagaaggtcaaagatgcacaccataagaaaaacttctggtttagctggttttcctaaacgagatgaaagtgctcatgatgcttttggtgctggacatagttctacaagcatctctgctggactaggtatggctgtcggtagagatttattagggaaaaccaacaacgtgatatcggtgatcggagatggcgccatgacggccggacgagcatatgaggcgataaataatgcaggatttcttgattcaaatctaatcgtcgttttaaacgacaacaagcaagtttcattaccgactgccacgttggacggacctgcaactcccgtcggggctctcagcggcgctttatccaaattgcaagccagtaccaagttccgtaagcttcgtgaagccgccaagagcattactaaacaaattggacctcaagcacatgaagtggcggcgaaagtcgacgaatacgcaagaggtatgattagtgctagcgggtcgactttattcgaggaactcggattatactacatcggtcccgtcgatggtcacaatgttgaagatttagtcaacatttttgaaaaagtcaagtcaatgcccgcacccggaccggttctaatccacatcgtgaccgaaaaaggcaaaggttaccctcctgctgaagccgctgctgaccgtatgcacggagttgtgaagtttgatgttccaactggaaaacaattcaagacaaaatcaccgacactttcgtatactcagtattttgctgaatcacttataaaagaagctgaagctgataacaagattgtcgcgatacacgccgccatgggaggcggtaccggactcaattacttccagaagaagtgtcctgaacgttgttttgatgtcggtatcgcggaacaacacgcagttactttcgccgcgggtttagccaccgaaggtcttaaaccattttgcgcgatctattcgtcgtttttgcaacgaggatacgatcaagtggtgcatgacgttgatctacaaaagttaccggttcggtttgcgatggaccgagctggtttagtcggggctgatggaccgacacattgtggtgcgtttgacataacctacatggcgtgtctaccaaacatggtggtgatggctccagccgatgaagccgaattgatgcacatggttgcaacggctgcagccattgacgacagaccgagttgctttcggttcccaagaggcaatggcattggtgcaccacttcctcctaataacaaagggattcccatagaggttggtaaaggaagaatattacttgaaggaactcgtgttgcgatattgggatacggttcgatagttcaagaatgtctaggtgcggctagcttgcttcaagcccataacgtgtctgcaaccgtagccgatgcgcggttctgcaaaccgttagacaccggactgattagacgattagccaacgagcatgaagtcttacttaccgtagaggaaggctcgattggtggatttggatcacacgttgctcactttctaagcttaaatggtctcttagatggaaaacttaagcttagagcaatgactcttcctgataaatacattgatcatggtgcaccacaagatcagcttgaagaagccggtctttcttcaaaacatatttgttcatctcttttatcacttttgggaaaacctaaagaagcacttcaatacaaatcaataatgtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 11 - DNA, 1557 bp, synthetic construct
  source 1..1557: mol_type = other DNA
atgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgattta

SEQ ID NO: 12 - DNA, 2199 bp, synthetic construct
  source 1..2199: mol_type = other DNA
atgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataa

SEQ ID NO: 13 - DNA, 735 bp, synthetic construct
  source 1..735: mol_type = other DNA
atgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaa

SEQ ID NO: 14 - DNA, 1842 bp, synthetic construct
  source 1..1842: mol_type = other DNA
atggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaa

SEQ ID NO: 15 - DNA, 1467 bp, synthetic construct
  source 1..1467: mol_type = other DNA
atgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaa

SEQ ID NO: 16 - DNA, 2904 bp, synthetic construct
  source 1..2904: mol_type = other DNA
atggcaaaccgtaagctggagaagatggcatctatcgacgttcacctgcgtcagctggttccaggtaaggtaagcgaggacgacaaactggttgaatacgacgctctgctgctggatcgtttcctggacattctgcaagacctgcacggtgaagacctgcgtgagactgtacaagagctgtacgaacattctgccgaatacgagggtaagcacgaaccgaagaaactggaagagctgggtagcgtgctgacctctctggacccaggtgattccattgttatcgctaaagcattcagccacatgctgaacctggctaacctggctgaagaagtgcagatcgcctaccgtcgtcgtatcaagaagctgaagaaaggtgacttcgttgatgagtcctctgctaccaccgagagcgacctggaggagactttcaagaaactggttggcgacctgaacaaatctccagaagaaatcttcgacgctctgaagaaccagactgtggatctggtactgactgcgcatccgacccaaagcgtacgtcgcagcctgctgcagaaacacggtcgtatccgtgactgcctggctcagctgtacgcgaaagacatcactccagatgacaaacaagaactggatgaagccctgcaacgtgaaatccaagcagcatttcgcaccgacgagatcaaacgtactccaccgactccacaggacgaaatgcgtgcaggtatgtcttacttccacgagactatctggaaaggtgtgccgaagttcctgcgtcgtgttgataccgctctgaagaacatcggtatcgaagaacgcgtgccgtacaacgctccgctgattcagtttagctcctggatgggtggtgatcgtgatggcaatccacgcgtaactccagaggtgactcgtgacgtgtgtctgctggctcgtatgatggctgcaaccatgtacttcaaccagattgaggacctgatgttcgagatgtccatgtggcgttgcaacgacgagctgcgtgcacgtgcagatgaagtacacgctaactctcgtaaagatgctgctaaacactacatcgagttctggaaatccatcccgaccaccgagccgtaccgtgttatcctgggtgacgttcgtgacaaactgtatcatacccgtgaacgtgcacaccaactgctgtctaacggtcacagcgatgtaccagttgaagcgaccttcatcaacctggagcaattcctggaaccgctggaactgtgctaccgttccctgtgttcttgtggtgatcgtccgatcgcagacggttccctgctggacttcctgcgtcaagtgtccaccttcggtctgagcctggttcgcctggacatccgtcaggagagcgaccgtcacactgacgtgctggacgccatcactacccacctggacatcggctcctatcgtgaatggtctgaagaacgtcgccaggagtggctgctgtctgaactgtctggtaaacgtccgctgttcggctccgacctgccgaagaccgaggaaatcgctgatgtgctggataccttccacgtgatcgcagaactgccagcagacagctttggtgcatacatcatcagcatggctactgctccatccgatgttctggctgttgaactgctgcaacgtgaatgccgtgtgaagcaaccgctgcgtgttgtaccactgtttgagaaactggcagacctggaggctgctccagctgccgttgctcgtctgttcagcgttgattggtacaagaatcgtatcaatggtaaacaggaggttatgatcggctattccgacagcggtaaagacgctggtcgtctgtctgctgcttggcagctgtacaaagcgcaggaagaactggttaaggtggcgaaagaatacggtgttaagctgaccatgtttcatggtcgtggtggtactgtaggtcgtggtggtggtccaacccacctggcgatcctgtctcaaccaccggataccatcaacggctctctgcgtgttaccgttcagggtgaagttatcgaacagagcttcggtgaggagcacctgtgcttccgtaccctgcaacgcttcaccgcagcaactctggaacacggtatgcgtccaccaatctctccgaaaccggaatggcgtgcactgctggatgagatggctgtggttgctaccgaagaataccgtagcgtagtgttccaggaaccacgtttcgttgaatactttcgcctggccactccagagctggaatacggtcgtatgaacattggctctcgtccaagcaaacgcaaaccgtccggtggcatcgaatccctgcgtgcaatcccgtggatcttcgcgtggactcagactcgcttccacctgccagtgtggctgggtttcggttccgctattcgtcacgtgatcgagaaagacgttcgcaacctgcacatgctgcaagacatgtaccaacactggccattcttccgtgttactatcgacctgatcgaaatggtgttcgctaaaggtgatccaggtattgcagcgctgtacgataagctgctggtgtctgaagagctgtggccattcggtgagaaactgcgtgctaacttcgaagagactaagaagctgatcctgcaaaccgctggccacaaagacctgctggaaggtgatccatatctgaaacaacgtctgcgtctgcgtgactcttacatcaccaccctgaatgtatgtcaggcgtacaccctgaaacgtatccgtgacccgtcctaccacgttaccctgcgtccgcatatctccaaagaaatcgctgagagcagcaaaccagctaaggagctgatcgaactgaacccgacctctgagtacgcaccaggcctggaggacactctgattctgaccatgaaaggcattgcagcaggtctgcagaacactggttaa

SEQ ID NO: 17 - DNA, 1869 bp, synthetic construct
  source 1..1869: mol_type = other DNA
atgccaccacagctgcaaaacggtctgaacctgagcgctaaagtggttcagggcagcctggattccctgccacaggctgttcgtgagttcctggagaacaacgcagagctgtgccaaccggatcacattcacatctgcgatggttccgaagaggagaacggtcgtctgctgggtcagatggaggaagaaggcatcctgcgtcgcctgaagaagtacgataactgttggctggctctgaccgatccacgtgatgtggctcgcatcgagtccaagactgttatcgttacccaagagcaacgcgatactgtgccgatcccgaagactggcctgagccagctgggtcgctggatgtctgaagaggactttgagaaagcgttcaacgctcgctttccaggttgcatgaaaggtcgtaccatgtacgtgatcccgttcagcatgggtccactgggttctccactgtctaagatcggcattgagctgactgactctccatacgttgttgcttctatgcgtatcatgacccgtatgggcactccagttctggaagcggttggtgatggcgagtttgtgaaatgtctgcactccgtgggctgtccactgccactgcagaaaccactggttaacaactggccatgcaacccagagctgaccctgatcgctcacctgccagatcgtcgtgaaatcatctcctttggttctggttatggtggtaactctctgctgggcaagaaatgcttcgctctgcgtatggcgagccgtctggcaaaggaagagggctggctggctgaacacatgctgattctgggcattaccaatccggagggtgagaagaagtacctggcagctgccttcccgtccgcttgtggtaagaccaatctggccatgatgaacccgtccctgccaggctggaaggtagagtgcgttggcgacgacattgcctggatgaagttcgatgcgcaaggccatctgcgtgccatcaatccggagaacggtttctttggtgttgctccaggtacttccgtaaagactaacccgaatgccatcaagaccatccagaagaacaccatcttcaccaacgtagcggaaacttccgacggtggcgtttactgggagggcattgatgaaccactggccagcggtgttactatcacctcctggaagaacaaagagtggagcagcgaagatggcgagccatgtgcccacccgaactctcgcttctgcactccagcctctcagtgtccaatcattgacgctgcgtgggagtctccagaaggcgttccgattgagggtatcatcttcggtggtcgtcgtccagcaggtgtgccactggtttacgaagctctgtcttggcagcacggtgtattcgttggtgctgcaatgcgctctgaggccaccgctgctgctgaacacaaaggcaagatcatcatgcacgatccgtttgcgatgcgtccgttcttcggttacaacttcggcaagtacctggctcactggctgagcatggctcagcatccagctgcgaaactgccgaagatttttcacgtaaactggtttcgtaaagacaaagaaggcaagttcctgtggccaggtttcggtgagaacagccgtgttctggagtggatgttcaatcgtatcgacggtaaagcatccaccaaactgactccgatcggctacatcccgaaggaggacgcactgaacctgaaaggtctgggtcatatcaacatgatggagctgttcagcatcagcaaagagttctgggagaaagaggttgaggacatcgagaagtacctggaggaccaggtgaacgcagacctgccgtgcgaaatcgaacgtgagatcctggcactgaaacagcgtatctctcaaatgtaa

SEQ ID NO: 18 - DNA, 17840 bp, synthetic construct
  source 1..17840: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggccaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttataattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataataacttaagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 19 - DNA, 23788 bp, synthetic construct
  source 1..23788: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttcctagggccaccatgactgaacttggttctgatactttgccctctgtgggccagtggcaacaaattctgactgcgccacccgtctggcaggtcaaggtgcagactgcatcctggacaagtgccgcgctggcgctgcgcgatcagggcgggcgtttgctgggattgtggggagaggcgggccatgaaaatgcatttttttacatacatgcgctgggccttggttcaaccggatatttgtggatcactctgacggtgcaatccagcgagggacaatttccctcgctggccgacatttttcccgctgcctcgcggatggagcgggccctgtacgatcttacgggaatcagggcggcggggaatgccgatacccggccatggctccgccatcagtgctggccagcggggatttttccactgcgggagggacaactggatggcagttccttcggcgtggatggtgatgccgattatccttttcaattaatcgatagcgtggatgtccaccagattccagtgggaccggtacacgcgggcaccattgagcccggacactttcgtttttcgtgtgtcggtgaacaaattctgcgtctggaagaacgattgggatacacccacaagggcgtcgaacgccttttccaagatcgggatgtgtttgcgggtgcgcgtctggcaggacgcatcagtggtgacactacagcgggttatgcctgggcctacagcatggcggtggaatccattgcccgatgcgaaatcccgcctcgtgcagccgctttgcgtgctgtttgcctggaacgggagcgcatggccaaccatctgggtgatcttgccgctctggggaatgacgctggcttcgccttctgtcagtcacaattcctgttcatcaaggaaagcctgctgcgcgaaaaccaggaaattttcggacaccgctatttgatggactgcattattcccggtggtgtagcttttgatctgaactccgcgcaaacagccgcgattcagcgtaacagtgaatcctggcagcgcaccgtgcagcgtttggacctcctcttgcaggagcactccggtctgcgggatcgtctggtgggtacaggaaaaattgtgccaccccgtgctgcagcactcggtatggcggggcttgccggccgcgccagtggtcaggcctgggatttgcgggtccagtttccccatgcaccttatcagaatctggatgtttccatgcagctttccccggaaggtgacgtggccgcacgctggcaattgcgctttcgggaattctatgagtctctgcgtctgcagacccagttgctgttggatctacccagtggagatgtcagcgcaaccattccggaaagcatccccgatggcgaaggcttgggcattgtcgaagcatggcgcggcgaggtctttgtcgccctatctgtggaaaacgcccgtattaaacgctgccatccccacgatccctcgtggaccctctggccggtccttgaggaggccgtgctgaacgatattgttgccgactttccgctgatcaataaatcgttcaatttgagttacagcggacatgatttataattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggcgcgccaccatgtcgactcctgacaatcacggcaagaaagcccctcaatttgctgcgttcaaaccgctaaccacggtacagaacgccaacgactgttgctgcgacggcgcatgttccagcacgccaactctctctgaaaacgtctccggcacccgctatagctggaaagtcagcggcatggactgcgccgcctgtgcgcgcaaggtagaaaatgccgtgcgccagcttgcaggcgtgaatcaggtgcaggtgttgttcgccaccgaaaaactggtggtcgatgccgacaatgacattcgtgcacaagttgaatctgcgctgcaaaaagcaggctattccctgcgcgatgaacaggccgccgaagaaccgcaagcatcacgcctgaaagagaatctgccgctgattacgctaatcgtgatgatggcaatcagctggggtctggagcagttcaatcatccgttcgggcaactggcgtttatcgcgaccacgctggttgggctgtacccgattgctcgtcaggcattacggttgatcaaatccggcagctacttcgccattgaaaccttaatgagcgtagccgctattggtgcactgtttattggcgcaacggctgaagctgcgatggtgttgctgctgtttttgattggtgaacgactggaaggctgggccgccagccgcgcgcgtcagggcgttagcgcgttaatggcgctgaaaccagaaaccgccacgcgcctgcgtaagggtgagcgggaagaggtggcgattaacagcctgcgccctggcgatgtgattgaagtcgccgcaggtgggcgtttgcctgccgacggtaaactgctctcaccgtttgccagttttgatgaaagcgccctgaccggcgaatccattccggtggagcgcgcaacgggcgataaagtccctgctggtgccaccagcgtagaccgtctggtgacgttggaagtgctgtcagaaccgggagccagcgccattgaccggattctgaaactgatcgaagaagccgaagagcgtcgcgctcccattgagcggtttatcgaccgtttcagccgtatctatacgcccgcgattatggccgtcgctctgctggtgacgctggtgccaccgctgctgtttgccgccagctggcaggagtggatttataaagggctgacgctgctgctgattggctgcccgtgtgcgttagttatctcaacgcctgcggcgattacctccgggctggcggcggcagcgcgtcgtggggcgttgattaaaggcggagcggcgctggaacagctgggtcgtgttactcaggtggcgtttgataaaaccggtacgctgaccgtcggtaaaccgcgcgttaccgcgattcatccggcaacgggtattagtgaatctgaactgctgacactggcggcggcggtcgagcaaggcgcgacgcatccactggcgcaagccatcgtacgcgaagcacaggttgctgaactcgccattcccaccgccgaatcacagcgggcgctggtcgggtctggcattgaagcgcaggttaacggtgagcgcgtattgatttgcgctgccgggaaacatcccgctgatgcatttactggtttaattaacgaactggaaagcgccgggcaaacggtagtgctggtagtacgtaacgatgacgtgcttggtgtcattgcgttacaggataccctgcgcgccgatgctgcaactgccatcagtgaactgaacgcgctgggcgtcaaaggggtgatcctcaccggcgataatccacgcgcagcggcggcaattgccggggagctggggctggagtttaaagcgggcctgttgccggaagataaagtcaaagcggtgaccgagctgaatcaacatgcgccgctggcgatggtcggtgacggtattaacgacgcgccagcgatgaaagctgccgccatcgggattgcaatgggtagcggcacagacgtggcgctggaaaccgccgacgcagcattaacccataaccacctgcgcggcctggtgcaaatgattgaactggcacgcgccactcacgccaatatccgccagaacatcactattgcgctggggctgaaagggatcttcctcgtcaccacgctgttagggatgaccgggttgtggctggcagtgctggcagatacgggggcgacggtgctggtgacagcgaatgcgttaagattgttgcgcaggagataataacttaagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggaattcatgacgaaaacgcgctcgaacatggcgcgctacggcaccagcctggccatcgtgctgggcgtgcacgtggtcgccgtggtgctgacgctcaactggtcggtgccccaggccatcgagctgccgccggcagcgatgatggtcgagttggcgccgttgccggagcccgcgccaccgccaccgcccaaggccgcgcccaagccaccggcagaggtcgaggagccgccgctgcccaagctggtggaggcccccaagccgaagatcgccatcgccaagccgcccaagcccaaggccaagccgcagccgcccaagcctgagaaaaagcctgagccgccgaaggacgaaccaccggccaaggacgatgtggcggataccccgccaagcaacgcgcagccgcagaaatcggccgcaccggcaccgagcatcgcctccaacagcaatgccctgcccagctggcagagcgacctgctgcgccacctggccaagtacaagaagtacccggaagacgctcgccgtcgcggcctgcagggcatcaaccgcctgcgcttcgtggtcgacgccgagggcaaggtagtctcgtactcgctggccggaggctcgggcagcgcggcgctggaccgggcgaccctggaaatgatccgtcgcgcaggctccgtaccgaagccgccagcggagctgttgaacaatggcacgatcgaagtcgtggcgccgttcgtctattccctggaccgacgctaagtttaaaccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaacccgggccaccatggcaaaccgtaagctggagaagatggcatctatcgacgttcacctgcgtcagctggttccaggtaaggtaagcgaggacgacaaactggttgaatacgacgctctgctgctggatcgtttcctggacattctgcaagacctgcacggtgaagacctgcgtgagactgtacaagagctgtacgaacattctgccgaatacgagggtaagcacgaaccgaagaaactggaagagctgggtagcgtgctgacctctctggacccaggtgattccattgttatcgctaaagcattcagccacatgctgaacctggctaacctggctgaagaagtgcagatcgcctaccgtcgtcgtatcaagaagctgaagaaaggtgacttcgttgatgagtcctctgctaccaccgagagcgacctggaggagactttcaagaaactggttggcgacctgaacaaatctccagaagaaatcttcgacgctctgaagaaccagactgtggatctggtactgactgcgcatccgacccaaagcgtacgtcgcagcctgctgcagaaacacggtcgtatccgtgactgcctggctcagctgtacgcgaaagacatcactccagatgacaaacaagaactggatgaagccctgcaacgtgaaatccaagcagcatttcgcaccgacgagatcaaacgtactccaccgactccacaggacgaaatgcgtgcaggtatgtcttacttccacgagactatctggaaaggtgtgccgaagttcctgcgtcgtgttgataccgctctgaagaacatcggtatcgaagaacgcgtgccgtacaacgctccgctgattcagtttagctcctggatgggtggtgatcgtgatggcaatccacgcgtaactccagaggtgactcgtgacgtgtgtctgctggctcgtatgatggctgcaaccatgtacttcaaccagattgaggacctgatgttcgagatgtccatgtggcgttgcaacgacgagctgcgtgcacgtgcagatgaagtacacgctaactctcgtaaagatgctgctaaacactacatcgagttctggaaatccatcccgaccaccgagccgtaccgtgttatcctgggtgacgttcgtgacaaactgtatcatacccgtgaacgtgcacaccaactgctgtctaacggtcacagcgatgtaccagttgaagcgaccttcatcaacctggagcaattcctggaaccgctggaactgtgctaccgttccctgtgttcttgtggtgatcgtccgatcgcagacggttccctgctggacttcctgcgtcaagtgtccaccttcggtctgagcctggttcgcctggacatccgtcaggagagcgaccgtcacactgacgtgctggacgccatcactacccacctggacatcggctcctatcgtgaatggtctgaagaacgtcgccaggagtggctgctgtctgaactgtctggtaaacgtccgctgttcggctccgacctgccgaagaccgaggaaatcgctgatgtgctggataccttccacgtgatcgcagaactgccagcagacagctttggtgcatacatcatcagcatggctactgctccatccgatgttctggctgttgaactgctgcaacgtgaatgccgtgtgaagcaaccgctgcgtgttgtaccactgtttgagaaactggcagacctggaggctgctccagctgccgttgctcgtctgttcagcgttgattggtacaagaatcgtatcaatggtaaacaggaggttatgatcggctattccgacagcggtaaagacgctggtcgtctgtctgctgcttggcagctgtacaaagcgcaggaagaactggttaaggtggcgaaagaatacggtgttaagctgaccatgtttcatggtcgtggtggtactgtaggtcgtggtggtggtccaacccacctggcgatcctgtctcaaccaccggataccatcaacggctctctgcgtgttaccgttcagggtgaagttatcgaacagagcttcggtgaggagcacctgtgcttccgtaccctgcaacgcttcaccgcagcaactctggaacacggtatgcgtccaccaatctctccgaaaccggaatggcgtgcactgctggatgagatggctgtggttgctaccgaagaataccgtagcgtagtgttccaggaaccacgtttcgttgaatactttcgcctggccactccagagctggaatacggtcgtatgaacattggctctcgtccaagcaaacgcaaaccgtccggtggcatcgaatccctgcgtgcaatcccgtggatcttcgcgtggactcagactcgcttccacctgccagtgtggctgggtttcggttccgctattcgtcacgtgatcgagaaagacgttcgcaacctgcacatgctgcaagacatgtaccaacactggccattcttccgtgttactatcgacctgatcgaaatggtgttcgctaaaggtgatccaggtattgcagcgctgtacgataagctgctggtgtctgaagagctgtggccattcggtgagaaactgcgtgctaacttcgaagagactaagaagctgatcctgcaaaccgctggccacaaagacctgctggaaggtgatccatatctgaaacaacgtctgcgtctgcgtgactcttacatcaccaccctgaatgtatgtcaggcgtacaccctgaaacgtatccgtgacccgtcctaccacgttaccctgcgtccgcatatctccaaagaaatcgctgagagcagcaaaccagctaaggagctgatcgaactgaacccgacctctgagtacgcaccaggcctggaggacactctgattctgaccatgaaaggcattgcagcaggtctgcagaacactggttaagcatgctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagccgcggccaccatgccaccacagctgcaaaacggtctgaacctgagcgctaaagtggttcagggcagcctggattccctgccacaggctgttcgtgagttcctggagaacaacgcagagctgtgccaaccggatcacattcacatctgcgatggttccgaagaggagaacggtcgtctgctgggtcagatggaggaagaaggcatcctgcgtcgcctgaagaagtacgataactgttggctggctctgaccgatccacgtgatgtggctcgcatcgagtccaagactgttatcgttacccaagagcaacgcgatactgtgccgatcccgaagactggcctgagccagctgggtcgctggatgtctgaagaggactttgagaaagcgttcaacgctcgctttccaggttgcatgaaaggtcgtaccatgtacgtgatcccgttcagcatgggtccactgggttctccactgtctaagatcggcattgagctgactgactctccatacgttgttgcttctatgcgtatcatgacccgtatgggcactccagttctggaagcggttggtgatggcgagtttgtgaaatgtctgcactccgtgggctgtccactgccactgcagaaaccactggttaacaactggccatgcaacccagagctgaccctgatcgctcacctgccagatcgtcgtgaaatcatctcctttggttctggttatggtggtaactctctgctgggcaagaaatgcttcgctctgcgtatggcgagccgtctggcaaaggaagagggctggctggctgaacacatgctgattctgggcattaccaatccggagggtgagaagaagtacctggcagctgccttcccgtccgcttgtggtaagaccaatctggccatgatgaacccgtccctgccaggctggaaggtagagtgcgttggcgacgacattgcctggatgaagttcgatgcgcaaggccatctgcgtgccatcaatccggagaacggtttctttggtgttgctccaggtacttccgtaaagactaacccgaatgccatcaagaccatccagaagaacaccatcttcaccaacgtagcggaaacttccgacggtggcgtttactgggagggcattgatgaaccactggccagcggtgttactatcacctcctggaagaacaaagagtggagcagcgaagatggcgagccatgtgcccacccgaactctcgcttctgcactccagcctctcagtgtccaatcattgacgctgcgtgggagtctccagaaggcgttccgattgagggtatcatcttcggtggtcgtcgtccagcaggtgtgccactggtttacgaagctctgtcttggcagcacggtgtattcgttggtgctgcaatgcgctctgaggccaccgctgctgctgaacacaaaggcaagatcatcatgcacgatccgtttgcgatgcgtccgttcttcggttacaacttcggcaagtacctggctcactggctgagcatggctcagcatccagctgcgaaactgccgaagatttttcacgtaaactggtttcgtaaagacaaagaaggcaagttcctgtggccaggtttcggtgagaacagccgtgttctggagtggatgttcaatcgtatcgacggtaaagcatccaccaaactgactccgatcggctacatcccgaaggaggacgcactgaacctgaaaggtctgggtcatatcaacatgatggagctgttcagcatcagcaaagagttctgggagaaagaggttgaggacatcgagaagtacctggaggaccaggtgaacgcagacctgccgtgcgaaatcgaacgtgagatcctggcactgaaacagcgtatctctcaaatgtaactcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggagctcaccatggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaaggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagggatccgccaccatgtctcaatccgtagaatcacggactcgaatcaaaaacgaacgttacgaatcaggtgttatcccttacgcaaaaatgggatactgggatgctgattacgttatcaaagatactgacgtacttgcaatgttccgtatgacacctcaaaaaggtgttgacccagttgaatgtgcagccgcaatcgcaggtgaatcttcaacagcaacttggacagttgtatggacagaccttctaacagcatgtgatctttaccgtgcaaaagcataccgtgttgacccagttcctggtgcaactgatcaatactttgcttacatcgcatacgaattagacctatttgaagaaggttctttagctaacttaacagcatcaattattggtaacgtattcggattcaaagcggtaaatgctttaagattagaagatatgcgtcttccaattgcatacctaaaaactttccaaggtccagcaactggtgtaattgtagaacgtgaaagattagacaaatatggtcgtcctttattaggtgcaacagttaaaccaaaattaggtctatctggtaaaaactatggtcgtgtagtttacgaaggtcttaaaggtggtctagatttccttaaagatgatgaaaacattaactctcaaccattcatgagatggaaagagcgtttcttattcggtatcgaaggtgtaaaccgtgctgctgctgctgctggtgaagtaaaaggtcactacttcaacgttactgctggtacaatggaagatatgtatgaacgtgctgaattctgtaaagaaatcggtagtgtaatctgtatgatcgaccttgtaatcggatatactgctattcaaagtatggcgatctgggcacgtaaaaacagtatgattcttcacttacaccgtgctggtaactctacatactcacgtcaaaaaacacatggtatgaacttccgtgtaatttgtaagtggatgcgtatggcaggtgttgaccacatacacgcaggtacagttgtaggtaaactagaaggggatcctttaatggttaaaggtttctataacacattattagaaacacaaacagatgtaaatcttgtacaaggtcttttctttgctcaagattgggcagcacttaacaaatgtatgccagttgcttcaggtggtattcactgtggtcaaatgcaccaacttattaactacttaggtgacgacgtagtattacaatttggtggtggtactattggtcacccagatggtatccaagctggtgctactgcaaaccgtgtagcacttgaatgtatggttgtagcacgtaacgaaggtcgtgactatgtaacagagggtcctcaaattcttcgtaatgcagctaagagctgtggacctcttcaaacagcattagatttatggaaagacattactttcaactatgcttctactgatacagctgacttcgtagaaactgcaacagctaacaagtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

SEQ ID NO: 20 - DNA, 890 bp, synthetic construct
  source 1..890: mol_type = other DNA
ggattcaaagaggagaaatactagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagctgcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaagctt

SEQ ID NO: 21 - DNA, 717 bp, synthetic construct
  source 1..717: mol_type = other DNA
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

(19) United States
(12) Patent Application Publication
(10) Pub. No.: US 2026/0226482 A1
(43) Pub. Date: Aug. 6, 2026

(54) PROTEIN DEVICES AND COMPOSITIONS FOR SUBLINGUAL ABSORPTION AND INGESTION

(71) Applicant: Bio Capital Holdings, LLC, Houston, TX (US)

(72) Inventor: Raul CUERO RENGIFO, Cypress, TX (US)

(21) Appl. No.: 19/424,257

(22) Filed: Dec. 18, 2025

Related U.S. Application Data
(60) Provisional application No. 63/736,782, filed on Dec. 20, 2024.

(51) Int. Cl. C12N 15/66; A61K 31/711

(52) U.S. Cl. CPC C12N 15/66; A61K 31/711; C12N 2800/102; C12N 2800/22

(57) ABSTRACT

Disclosed herein are DNA constructs and biological devices comprising S. cerevisiae cells and extracts and lysates produced therefrom, wherein the DNA constructs encode genes for complete proteins including, but not limited to, casein, ovalbumin or a fragment thereof, lactalbumin, and glycomacropeptide (GMP). The extracts and lysates from these devices can be used alone in oral dosage forms for nutritional supplementation or can be used in combination with other lysates and extracts containing vitamins such as carotenoids, organic electrolytes, flavorants such as steviol glycosides, and the like. Also disclosed herein are oral dosage forms including sublingual and buccal dosage forms comprising the disclosed extracts and lysates. In some aspects, the disclosed lysates and extracts can be encapsulated in hydrogel microparticles in order to facilitate absorption. In some aspects, the disclosed extracts and lysates can be added to food and beverage products to improve the nutritional profiles of the same.

### CROSS-REFERENCE TO RELATED APPLICATIONS

This application claims the benefit of U.S. Provisional Application No. 63/736,782, filed on Dec. 20, 2024, which is incorporated herein by reference in its entirety.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The sequence listing in written computer readable format (CRF) as a text file named “930201-8170_Sequence_Listing.xml” created on Dec. 11, 2024, and having a size of 114,948 bytes, is incorporated by reference in its entirety.

### BACKGROUND

All proteins synthesized in humans are made of 20 amino acids. Out of these 20, 9 are classified as essential amino acids because they cannot be synthesized by humans and must be supplied from exogenous diet. Proteins rich in the 9 essential amino acids are known as complete proteins; supplemental nutrition products frequently include one or more complete proteins.

Certain individuals may need supplemental nutrition for a variety of medical reasons. For people with neurological or other conditions that impair swallowing or otherwise cause dysphagia (e.g., stroke, amyotrophic lateral sclerosis, and/or Parkinson's disease), forms of supplemental nutrition capable of being sublingually absorbed may be particularly important. However, individuals with gastrointestinal disease or reduced stomach capacity due to bariatric surgery, may benefit from additional proteins and vitamins in non-bulky or low-volume forms. Cancer patients and others experiencing nausea may benefit from nutrition sources that can be absorbed sublingually. Additionally, some individuals with dietary restrictions such as vegans may wish for complete protein sources without consuming products produced by animals, and some athletes may wish for additional protein for performance purposes without consuming bulky foods and large volumes of liquid.

In addition to amino acids, other compounds such as vitamins, coenzymes, and minerals may be of benefit to consumers requiring supplemental nutrition. For example, antioxidants such as, for example, vitamins A and C may help to prevent the breakdown of proteins, or may stimulate the development of lipids and carbohydrates.

It would be advantageous to develop new supplemental forms of nutrition that include all 9 essential amino acids and, optionally, additional vitamins and minerals. It would further be advantageous if these nutritional supplements could be absorbed in the oral cavity through digestion by saliva proteases or through sublingual absorption. It would also be advantageous if these nutritional supplements could be consumed alone or could be added to other food and beverage products. It would further be advantageous if the proteins and/or other nutritional compounds in these nutrition supplements could be produced inexpensively and quickly, in high volumes. The present invention addresses these needs.

### SUMMARY

In one aspect, disclosed herein are DNA constructs and biological devices comprising S. cerevisiae cells and extracts and lysates produced therefrom, wherein the DNA constructs encode genes for complete proteins including, but not limited to, casein, ovalbumin or a fragment thereof, lactalbumin, and glycomacropeptide (GMP). The extracts and lysates from these devices can be used alone in oral dosage forms for nutritional supplementation or can be used in combination with other lysates and extracts containing vitamins such as carotenoids, organic electrolytes, flavorants such as steviol glycosides, and the like. Also disclosed herein are oral dosage forms including sublingual and buccal dosage forms comprising the disclosed extracts and lysates. In some aspects, the disclosed lysates and extracts can be encapsulated in hydrogel microparticles in order to facilitate absorption. In some aspects, the disclosed extracts and lysates can be added to food and beverage products to improve the nutritional profiles of the same.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

Many aspects of the present disclosure can be better understood with reference to the following drawings, which are incorporated in and constitute a part of this specification. The components in the drawings are not necessarily to scale, emphasis instead being placed upon clearly illustrating the principles of the present disclosure.

FIGS. 1A-1B show, respectively, linear and circular maps of an exemplary biological device for producing complete proteins including casein, an ovalbumin fragment, lactalbumin, and glycomacropeptide.

FIGS. 2A-2B show, respectively, linear and circular maps of an exemplary biological device for producing carotenoids useful in the disclosed compositions.

FIGS. 3A-3B show, respectively, linear and circular maps of a second exemplary biological device for producing carotenoids useful in the disclosed compositions.

FIGS. 4A-4B show, respectively, linear and circular maps of an exemplary biological device for producing organic electrolytes useful in the disclosed compositions.

FIGS. 5A-5B show, respectively, linear and circular maps of a second exemplary biological device for producing organic electrolytes useful in the disclosed compositions.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are biological devices and methods to produce four different complete proteins, containing the nine essential amino acids for high nutritional value. In one aspect, the proteins and supplements containing the same can be broken down by saliva sublingually. In one aspect, it is known that saliva contains protease enzymes including, but not limited to, cathepsins, serine proteases, peptidase, and others, that are able to break down proteins. Also disclosed herein are protein particles having nanoparticle-scale sizes, facilitating sublingual absorption. Although the extracts and lysates disclosed herein can be sublingually absorbed, in one aspect, they can also be ingested and absorbed in the gastrointestinal tract, either alone or as components of foods and beverages to which they are added. In another aspect, in addition to being used as a nutritional supplement for individuals in need thereof, the present extracts and lysates can be used by athletes or others wishing to build muscle strength, or can be used to enhance health such as for the anti-inflammatory properties of the proteins and vitamins supplied by the extracts and lysates, or can be used to support the immune system. In some aspects, the extracts and lysates contain vitamin C and can aid in collagen production, which may also be useful to support healthy joint function.

Disclosed herein are DNA constructs containing the following genetic components:
(a) a gene that encodes casein;
(b) a gene that encodes ovalbumin or a fragment thereof;
(c) a gene that encodes lactalbumin;
(d) a gene that encodes glycomacropeptide (GMP).

The DNA constructs may variously encode genes encoding reporter proteins, genes encoding resistance to one or more antibiotics, and the like, and may include regulatory sequences including promoters, terminators, ribosomal binding sites, LAC operons, or other components necessary for the replication of and expression of the genes encoded by the DNA constructs inside microbial hosts such as, for example, Saccharomyces cerevisiae, Escherichia coli, and other microorganisms. Also disclosed are vectors including the DNA constructs and biological devices consisting of host cells that include one or more copies of the vectors. In some aspects, the DNA constructs can include salivary enzymes such as, for example, cathepsins, serine proteases, peptidases, and combinations thereof.

Described herein are microbial cultures and extracts containing complete proteins and methods of making and using thereof. In one aspect, the method of making a disclosed culture or extract includes the steps of (a) making a DNA construct containing genes for producing a casein, ovalbumin or a fragment thereof, lactalbumin, and GMP, (b) introducing the DNA construct into host microbial cells via transformation or transfection, and (c) culturing the microbial cells to produce the protein-containing cultures and extracts. The cultures are grown in standard media for host cells such as, for example, S. cerevisiae. The compositions of these cultures and extracts can be tailored to have specific properties such as, for example, the ability to provide nutrition in the form of protein, vitamins, and/or minerals to patients in need thereof.

Before the present compounds, compositions, articles, devices, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In this specification and in the claims that follow, reference will be made to a number of terms that shall defined to have the following meanings:

It must be noted that, as used in the specification and the appended claims, the singular forms “a,” “an,” and “the” include plural referents unless the context clearly dictates otherwise. Thus, for example, reference to “a metabolite” includes mixtures of two or more such metabolites, and the like.

“Optional” or “optionally” means that the subsequently described event or circumstance can or cannot occur, and that the description includes instances where the event or circumstance occurs and instances where it does not. For example, the phrase “a microorganism is optionally genetically modified” means that the microorganism may or may not be genetically modified.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

As used herein, the term “about” is used to provide flexibility to a numerical range endpoint by providing that a given numerical value may be “a little above” or “a little below” the endpoint without affecting the desired result. For purposes of the parent disclosure, “about” refers to a range extending from 10% below the numerical value to 10% above the numerical value. For example, if the numerical value is 10, “about 10” means between 9 and 11, inclusive of the endpoints 9 and 11.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed that while specific reference to each various individual and collective combination and permutation of these compounds may not be explicitly disclosed, each is specifically contemplated and described herein. For example, if a complete protein is disclosed and discussed and a number of different additional vitamins are discussed, each and every combination and permutation of complete protein and additional vitamin that is possible is specifically contemplated unless specifically indicated to the contrary. For example, if a class of molecules A, B, and C are disclosed as well as a class of molecules D, E, and F, and an example of a combination molecule, A-D, is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the subgroup of A-E, B-F, and C-E is specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed, it is understood that each of these additional steps can be performed with any specific embodiment or combination of elements of the disclosed methods, and that each such combination is specifically contemplated and should be considered disclosed.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

“Carotenoids” as used herein are red, yellow, and orange hydrophobic pigments. Carotenoids are important plant metabolites from numerous agricultural and other crops including, but not limited to, tomatoes, carrots, and watermelon. In one aspect, the processes disclosed herein are useful for increasing the production of carotenoids from plants, plant calluses, and/or microorganisms engineered to produce carotenoids. In one aspect, carotenoids can be useful in numerous aspects of human health including maintaining vision and fighting cancer. Lycopene is one example of a carotenoid. In one aspect, the biological devices disclosed in U.S. Pat. No. 9,828,609 and international patent application publication WO2019/055326 can be used to produce carotenoids including, but not limited to, lycopene.

“Essential amino acids” as used herein include those that humans cannot produce metabolically either at all, or not in sufficient amounts to satisfy their nutritional needs. Essential amino acids, in one aspect, are required to be supplied by dietary sources. In a further aspect, the disclosed nutritional compositions include at least one complete protein containing all nine essential amino acids. The nine essential amino acids include histidine, isoleucine, leucine, lysine, methionine, phenylalanine, threonine, tryptophan, and valine.

“Nicotinamide adenine dinucleotide” or “NAD” is a coenzyme. In some aspects, NAD may be phosphorylated to form NADP. Through redox reactions, NAD switches from an oxidized (NAD(P)⁺) to a reduced (NAD(P)H) form and back, and is thus a key component of many electron transfer reactions in the body. In one aspect, NAD is important to many metabolic reactions including, but not limited to, production of lycopene, in post-translational modification of proteins, in protein production, and the like. In one aspect, the nutritional compositions disclosed herein include NAD or NADP. Further in this aspect, the NAD(P) can enhance absorption of the proteins or other molecules in the compositions, or can act as a carrier for the same. In an alternative aspect, the NAD(P) can be taken separately to enhance the effects of the nutritional compositions.

### DNA Constructs and Biological Devices

In one aspect, cells transformed with a DNA construct can be used in the methods described herein. It is understood that one way to define the variants and derivatives of the genetic components and DNA constructs described herein is in terms of homology/identity to specific known sequences. Those of skill in the art readily understand how to determine the homology of two nucleic acids. For example, the homology can be calculated after aligning two sequences so that the homology is at its highest level. Another way of calculating homology can be performed according to published algorithms (see Zuker, M., Science, 244:48-52, 1989; Jaeger et al, Proc. Natl. Acad. Sci. USA, 86:7706-7710, 1989; Jaeger et al, Methods Enzymol., 183:281-306, 1989, which are herein incorporated by reference for at least material related to nucleic acid alignment).

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of mutations and homology can be combined together in any combination, such as embodiments that have at least about 70%, about 75%, about 80%, about 85%, about 90%, about 95%, or about 99% homology to a particular sequence wherein the variants are conservative or silent mutations. It is understood that any of the sequences described herein can be a variant or derivative having the homology values listed above.

In some aspects, genes of interest can be spliced into suitable vectors using restriction enzymes and/or other techniques known in the art. Further in this aspect, synthesis and/or isolation of the genes of interest prior to inclusion in the disclosed constructs may result in the addition of C-terminal and/or N-terminal sequence data including, but not limited to, restriction enzyme recognition sites, linking bases, short segments of chromosomal DNA (including introns or portions of introns if the sequences originate from eukaryotic cells), transposons, nucleotide repeats, regulatory sequences, and/or other material that do not contribute to the known structure of the expressed protein, or are not part of the expressed protein's active site. In one aspect, presence of these remnants may lead to somewhat reduced homology with respect to gene sequence, but the DNA constructs encoding the same can still produce proteins having the desired sequence, active site, and function.

In another aspect, many eukaryotic genes include introns and mRNAs produced during transcription of the same can be spliced differently, producing several transcript variants from the same gene but having slightly different sequences (i.e., reduced levels of homology). In one aspect, different transcript variants can produce proteins having the same active site but differing in another way (e.g. in C-terminal or N-terminal sequence, affecting assembly of protein subunits or other folding processes, cellular localization of the peptides or proteins, or activity level of the peptides or proteins produced due to differential regulation, or the like.

In one aspect, a database such as, for example, GenBank, can be used to determine the sequences of genes and/or regulatory regions of interest, the species from which these elements originate, and related homologous sequences.

In one aspect, the nucleic acids used in the DNA constructs described herein can be amplified using polymerase chain reaction (PCR) prior to being ligated into a plasmid or other vector. Typically, PCR-amplification techniques make use of primers, or short, chemically-synthesized oligonucleotides that are complementary to regions on each respective strand flanking the DNA or nucleotide sequence to be amplified. A person having ordinary skill in the art will be able to design or choose primers based on the desired experimental conditions. In general, primers should be designed to provide for both efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the skilled practitioner with primer design. Primers can optionally incorporate restriction enzyme recognition sites at their 5′ ends to assist in later ligation into plasmids or other vectors.

PCR can be carried out using purified DNA, unpurified DNA that is integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase, such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or genes. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In a further aspect, the plasmid can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of that coding sequence.

As used herein, “expression” refers to transcription and/or accumulation of an mRNA derived from a gene or DNA fragment. Expression may also be used to refer to translation of mRNA into a peptide, polypeptide, or protein.

### Protein-Producing DNA Constructs

In one aspect, provided herein are DNA constructs having at least the following genetic components:
(a) a gene that encodes casein;
(b) a gene that encodes ovalbumin or a fragment thereof;
(c) a gene that encodes lactalbumin; and
(d) a gene that encodes glycomacropeptide (GMP).

Each component of the DNA constructs is described in detail below. The components can be present in any order.

In one aspect, the DNA constructs disclosed herein incorporate a gene that encodes an ovalbumin. In a further aspect, ovalbumin is the main protein component of egg white. Although ovalbumin shows some homology to serpins, it is typically characterized as a storage protein and does not have serine protease inhibitory activity as do other serpins. Chicken ovalbumin has 385 amino acids and may adopt different isoforms depending on phosphorylation level; sequences of ovalbumin from other avian species may vary slightly. Heating of ovalbumin causes an irreversible conformational change. Ovalbumin contains all nine essential amino acids. In some aspects, only a fragment of ovalbumin is used, such as, for example, that fraction represented by amino acids 242 through 377 in the full ovalbumin sequence. In a further aspect, this fragment is especially rich in essential amino acids.

In one aspect, the gene that encodes an ovalbumin fragment is isolated from domestic chicken. In a further aspect, the gene that encodes an ovalbumin fragment has SEQ ID NO. 1 or at least 70% homology thereto, at least 75% homology thereto, at least 80% homology thereto, at least 85% homology thereto, at least 90% homology thereto, at least 95% homology thereto, or at least 99% homology thereto.

Other sequences encoding ovalbumin, a fragment thereof, or related or homologous genes can be identified in a database such as, for example, GenBank. In one aspect, the gene that encodes an ovalbumin fragment is isolated from Gallus gallus and can be identified by the GI number MF321665.1 in the GenBank database. In another aspect, sequences useful herein include those with GI numbers listed in Table 1:

TABLE 1
Ovalbumin

| Source Organism | Sequence Description | GI Number |
|---|---|---|
| Gallus gallus | ovalbumin | MF321665.1 |
| Gallus gallus | ovalbumin | MF321601.1 |
| Gallus gallus | ovalbumin | MF321599.1 |
| Gallus gallus | ovalbumin | MF321727.1 |
| Gallus gallus | ovalbumin | MF321477.1 |
| Gallus gallus | ovalbumin | MF321560.1 |
| Gallus gallus | ovalbumin | MF321582.1 |
| Gallus gallus | ovalbumin | MF321711.1 |
| Gallus gallus | ovalbumin | MF321714.1 |
| Gallus gallus | ovalbumin | MF321597.1 |
| Gallus gallus | ovalbumin | MF321480.1 |
| Gallus gallus | ovalbumin | MF321465.1 |
| Gallus gallus | ovalbumin | MF321687.1 |
| Gallus gallus | ovalbumin | MF321625.1 |
| Gallus gallus | ovalbumin | MF321659.1 |
| Gallus gallus | ovalbumin | MF321611.1 |
| Gallus gallus | ovalbumin | MF321579.1 |
| Gallus gallus | ovalbumin | MF321571.1 |
| Gallus gallus | ovalbumin | MF321525.1 |
| Gallus gallus | ovalbumin | MF321698.1 |
| Gallus gallus | ovalbumin | MF321568.1 |
| Gallus gallus | ovalbumin | MF321617.1 |
| Gallus gallus | ovalbumin | MF321529.1 |
| Gallus gallus | ovalbumin | MF321646.1 |
| Gallus gallus | ovalbumin | MF321694.1 |
| Gallus gallus | ovalbumin | MF321493.1 |
| Gallus gallus | ovalbumin | MF321491.1 |
| Gallus gallus | ovalbumin | MF321712.1 |
| Gallus gallus | ovalbumin | MF321649.1 |
| Gallus gallus | ovalbumin | MF321657.1 |
| Gallus gallus | ovalbumin | MF321685.1 |
| Gallus gallus | ovalbumin | MF321499.1 |
| Gallus gallus | ovalbumin | MF321459.1 |
| Gallus gallus | ovalbumin | MF321596.1 |
| Gallus gallus | ovalbumin | MF321719.1 |
| Gallus gallus | ovalbumin | MF321700.1 |
| Gallus gallus | ovalbumin | MF321546.1 |
| Gallus gallus | ovalbumin | MF321451.1 |
| Gallus gallus | ovalbumin | MF321725.1 |
| Gallus gallus | ovalbumin | MF321600.1 |
| Gallus gallus | ovalbumin | MF321676.1 |
| Gallus gallus | ovalbumin | MF321682.1 |
| Gallus gallus | ovalbumin | MF321447.1 |
| Gallus gallus | ovalbumin | MF321717.1 |
| Gallus gallus | ovalbumin | MF321728.1 |
| Gallus gallus | ovalbumin | MF321524.1 |
| Gallus gallus | ovalbumin | MF321501.1 |
| Gallus gallus | ovalbumin | MF321684.1 |
| Gallus gallus | ovalbumin | MF321595.1 |
| Gallus gallus | ovalbumin | MF321713.1 |
| Gallus gallus | ovalbumin | MF321552.1 |
| Gallus gallus | ovalbumin | MF321628.1 |
| Gallus gallus | ovalbumin | MF321589.1 |
| Gallus gallus | ovalbumin | MF321527.1 |
| Gallus gallus | ovalbumin | MF321475.1 |
| Gallus gallus | ovalbumin | MF321695.1 |
| Gallus gallus | ovalbumin | MF321515.1 |
| Gallus gallus | ovalbumin | MF321683.1 |
| Gallus gallus | ovalbumin | MF321466.1 |
| Gallus gallus | ovalbumin | MF321456.1 |
| Gallus gallus | ovalbumin | MF321490.1 |
| Gallus gallus | ovalbumin | MF321543.1 |
| Gallus gallus | ovalbumin | MF321631.1 |
| Gallus gallus | ovalbumin | MF321645.1 |
| Gallus gallus | ovalbumin | NM_205152.3 |
| Gallus gallus | ovalbumin | MH360741.1 |
| Gallus gallus | ovalbumin | MF321530.1 |
| Gallus gallus | ovalbumin | MF321635.1 |
| Gallus gallus | ovalbumin | MF321644.1 |
| Gallus gallus | ovalbumin | MF321492.1 |
| Gallus gallus | ovalbumin | MF321545.1 |
| Gallus gallus | ovalbumin | MF321606.1 |
| Gallus gallus | ovalbumin | MF321630.1 |
| Gallus gallus | ovalbumin | MF321487.1 |
| Gallus gallus | ovalbumin | MF321476.1 |
| Gallus gallus | ovalbumin | MF321636.1 |
| Gallus gallus | ovalbumin | MF321488.1 |
| Gallus gallus | ovalbumin | MF321584.1 |
| Gallus gallus | ovalbumin | MF321669.1 |
| Gallus gallus | ovalbumin | MF321660.1 |
| Gallus gallus | ovalbumin | MF321564.1 |
| Gallus gallus | ovalbumin | MF321656.1 |
| Gallus gallus | ovalbumin | MF321455.1 |
| Gallus gallus | ovalbumin | MF321495.1 |
| Gallus gallus | ovalbumin | MF321581.1 |
| Gallus gallus | ovalbumin | MF321720.1 |
| Gallus gallus | ovalbumin | MF321544.1 |
| Gallus gallus | ovalbumin | MF321647.1 |
| Gallus gallus | ovalbumin | MF321485.1 |
| Gallus gallus | ovalbumin | MF321587.1 |
| Gallus gallus | ovalbumin | MF321567.1 |
| Gallus gallus | ovalbumin | MF321654.1 |
| Gallus gallus | ovalbumin | MH360742.1 |
| Gallus gallus | ovalbumin | MF321681.1 |
| Gallus gallus | ovalbumin | MF321653.1 |
| Gallus gallus | ovalbumin | MF321460.1 |
| Gallus gallus | ovalbumin | MF321511.1 |
| Gallus gallus | ovalbumin | MF321621.1 |
| Gallus gallus | ovalbumin | MF321514.1 |
| Gallus gallus | ovalbumin | MF321565.1 |

In one aspect, the DNA constructs disclosed herein include a gene that expresses glycomacropeptide. In some aspects, glycomacropeptide is isolated from the liquid by-product of cheese production known as whey. In an aspect, glycomacropeptide is formed when casein micelles are cleaved by chymosin. Thus, in some aspects, glycomacropeptide is referred to as kappa-casein or a variant of this name. In some cases, “whey protein” is used to refer to glycomacropeptide; however, whey protein includes α-lactalbumin, β-lactoglobulin, serum albumin, and various immunoglobulin proteins. Whey can make up about 20% of cow's milk and 60-80% of human milk. In one aspect, glycomacropeptide (GMP) is a peptide lacking secondary structure that is soluble in water. In one aspect, glycomacropeptide is not considered a complete protein source since it lacks the aromatic amino acids phenylalanine and tryptophan. In another aspect, the casein can be a kappa 1 casein or a kappa 2 casein, or can be a casein or casein analog expressed by a natural or genetically modified plant such as, for example, soy.

In one aspect, the gene that encodes glycomacropeptide is isolated from domestic cattle. In a further aspect, the gene that encodes glycomacropeptide has SEQ ID NO. 2 or at least 70% homology thereto, at least 75% homology thereto, at least 80% homology thereto, at least 85% homology thereto, at least 90% homology thereto, at least 95% homology thereto, or at least 99% homology thereto.

Other sequences encoding glycomacropeptide or related or homologous genes can be identified in a database such as, for example, GenBank. In one aspect, the gene that encodes glycomacropeptide is isolated from Bos taurus and can be identified by the GI number X00565.1 in the GenBank database. In another aspect, sequences useful herein include those with GI numbers listed in Table 2:

TABLE 2
Glycomacropeptide

| Source Organism | Sequence Description | GI Number |
|---|---|---|
| Bos taurus | kappa-casein | X00565.1 |
| Bos taurus | kappa-casein | NM_174294.2 |
| Bos taurus | kappa-casein | KP897162.1 |
| Bos taurus | kappa-casein | AJ841942.1 |
| Bos taurus | kappa-casein | AY380228.1 |
| Bos taurus | kappa-casein | AF123250.1 |
| Bos taurus | kappa-casein | KP897163.1 |
| Bos indicus | kappa-casein | EU595509.1 |
| Bos taurus | kappa-casein | HQ589917.1 |
| Bos taurus | kappa-casein | X14908.1 |
| Bos taurus | kappa-casein | AJ841944.1 |
| Bos indicus | kappa-casein | EU595507.1 |
| Bos indicus x Bos taurus | kappa-casein | AF121023.1 |
| Bos indicus x Bos taurus | kappa-casein | KR149430.1 |
| Bos grunniens | kappa-casein | GU441771.1 |
| Bos indicus | kappa-casein | HQ589922.1 |
| Bison bonasus | kappa-casein | AF030325.1 |
| Bos indicus | kappa-casein | KF571746.1 |
| Bos javanicus | kappa-casein | JX862172.1 |
| Bison bonasus | kappa-casein | U10379.1 |
| Bos indicus | kappa-casein | OL330808.1 |
| Bos indicus | kappa-casein | OM142644.1 |
| Bos taurus | kappa-casein | AF105260.1 |
| Bos taurus | kappa-casein | HQ589916.1 |
| Bos indicus | kappa-casein | HQ589918.1 |
| Bos gaurus | kappa-casein | AF030323.1 |
| Bos indicus x Bos taurus | kappa-casein | XM_027544672.1 |
| Bos indicus | kappa-casein | OL439895.1 |
| Bos indicus | kappa-casein | OL653986.1 |
| Bos indicus | kappa-casein | HQ589914.1 |
| Bos gaurus | kappa-casein | JX862171.1 |
| Bos indicus | kappa-casein | MG581713.1 |
| Bos taurus | kappa-casein | AF123251.1 |
| Bos indicus | kappa-casein | OM142641.1 |
| Bos indicus | kappa-casein | PP421165.1 |
| Bos frontalis | kappa-casein | EU595506.1 |
| Bos indicus | kappa-casein | EU595511.1 |
| Bos indicus | kappa-casein | OM142647.1 |
| Bos taurus | kappa-casein | AJ841941.1 |
| Bos javanicus | kappa-casein | AF030324.1 |
| Bos indicus x Bos taurus | kappa-casein | AF092513.1 |
| Bos javanicus | kappa-casein | XM_061420394.1 |
| Bos indicus | kappa-casein | OL653985.1 |
| Bos indicus | kappa-casein | AY367769.1 |
| Bos frontalis | kappa-casein | GU991380.1 |
| Bos indicus | kappa-casein | OM142643.1 |
| Bos indicus | kappa-casein | EU295526.1 |
| Bos indicus | kappa-casein | OM142642.1 |
| Bos indicus | kappa-casein | XM_019962873.1 |
| Bos indicus | kappa-casein | OM142645.1 |
| Bos indicus | kappa-casein | OM142640.1 |
| Bos indicus | kappa-casein | OM142639.1 |
| Bos taurus | kappa-casein | AJ849456.1 |
| Bison bonasus | kappa-casein | JX862168.1 |
| Bos indicus | kappa-casein | EU595512.1 |
| Bos taurus | kappa-casein | JX862176.1 |
| Bos taurus | kappa-casein | AJ619772.1 |
| Bison bison | kappa-casein | JX862167.1 |
| Bos grunniens | kappa-casein | EF565131.1 |
| Bos indicus | kappa-casein | KY368689.1 |
| Bos grunniens | kappa-casein | AY095312.1 |
| Bos indicus | kappa-casein | EU595513.1 |
| Bos indicus | kappa-casein | HQ589915.1 |
| Bos indicus | kappa-casein | KR149429.1 |
| Bison bison athabascae | kappa-casein | U37511.1 |
| Bos indicus | kappa-casein | HQ589919.1 |
| Bos taurus | kappa-casein | MK455075.1 |
| Bos taurus | kappa-casein | BC102120.1 |
| Bison bison bison | kappa-casein | XM_010839113.1 |
| Bos taurus | kappa-casein | AY380229.1 |
| Bos indicus | kappa-casein | AY367770.1 |
| Bison bison bison | kappa-casein | U37510.1 |
| Bos taurus | kappa-casein | HQ589920.1 |
| Bos taurus | kappa-casein | EF378700.1 |
| Bos indicus | kappa-casein | OM142646.1 |
| Bos indicus | kappa-casein | EU595508.1 |
| Bos frontalis | kappa-casein | HQ728337.1 |
| Bison bison | kappa-casein | AF030322.1 |
| Bison bison bison | kappa-casein | XM_010839114.1 |
| Bos indicus | kappa-casein | HQ589921.1 |
| Bos indicus | kappa-casein | JX862175.1 |
| Bos indicus | kappa-casein | KY368691.1 |
| Bos grunniens | kappa-casein | JQ979053.1 |
| Bos indicus | kappa-casein | KY368690.1 |
| Bos grunniens | kappa-casein | AH009225.2 |
| Bos indicus | kappa-casein | EU595510.1 |
| Bos taurus | kappa-casein | EF133462.1 |
| Bos indicus | kappa-casein | EU365833.1 |
| Bos taurus | kappa-casein | M36641.1 |
| Bos gaurus | kappa-casein | JX862170.1 |
| Bos taurus | kappa-casein | M38333.1 |
| Bos taurus | kappa-casein | U84250.1 |
| Bos taurus | kappa-casein | AF041482.1 |
| Bos indicus | kappa-casein | EU365834.1 |
| Bos javanicus | kappa-casein | U84252.1 |
| Bos grunniens | kappa-casein | MH378281.1 |
| Bos mutus | kappa-casein | XM_014478623.1 |
| Bos mutus | kappa-casein | XM_005897042.2 |
| Bos mutus | kappa-casein | JX862174.1 |
| Bos grunniens | kappa-casein | ON100504.1 |

In one aspect, the DNA constructs disclosed herein incorporate a gene that expresses lactalbumin. Lactalbumin is a component of whey protein. α-lactalbumin regulates the production of lactose in the milk of mammals as the regulatory subunit of the lactose synthase heterodimer. α-lactalbumin is the most abundant whey protein in human milk and is essential for the nutrition of newborns. α-lactalbumin includes cysteine, tryptophan, and branched chain amino acids such as leucine, valine, and isoleucine.

In one aspect, the gene that encodes lactalbumin is isolated from domestic cattle. In a further aspect, the gene that encodes lactalbumin has SEQ ID NO. 3 or at least 70% homology thereto, at least 75% homology thereto, at least 80% homology thereto, at least 85% homology thereto, at least 90% homology thereto, at least 95% homology thereto, or at least 99% homology thereto.

Other sequences encoding lactalbumin or related or homologous genes can be identified in a database such as, for example, GenBank. In one aspect, the gene that encodes lactalbumin is isolated from Bos taurus and can be identified by the GI number BC102173.1 in the GenBank database. In another aspect, sequences useful herein include those with GI numbers listed in Table 3:

TABLE 3
Lactalbumin

| Source Organism | Sequence Description | GI Number |
|---|---|---|
| Bos taurus | alpha-lactalbumin | BC102173.1 |
| Bos taurus | alpha-lactalbumin | NM_174378.2 |
| Bos taurus | alpha-lactalbumin | M18780.1 |
| Bos taurus | alpha-lactalbumin | BT025469.1 |
| Bos taurus | alpha-lactalbumin | FJ232912.1 |
| Bubalus kerabau | alpha-lactalbumin | XM_055566327.1 |
| Bos indicus x Bos taurus | alpha-lactalbumin | XM_027543629.1 |
| Bos grunniens | alpha-lactalbumin | GU562877.1 |
| Bos mutus | alpha-lactalbumin | XM_005896365.2 |
| Bubalus bubalis | alpha-lactalbumin | KX896654.1 |
| Bos javanicus | alpha-lactalbumin | XM_061416302.1 |
| Bison bison bison | alpha-lactalbumin | XM_010857725.1 |
| Bos indicus | alpha-lactalbumin | XM_019960037.1 |
| Capra hircus | alpha-lactalbumin | NM_001285635.1 |
| Bubalus bubalis | alpha-lactalbumin | MT130465.1 |
| Bubalus bubalis | alpha-lactalbumin | NM_001290936.1 |
| Ovis canadensis | alpha-lactalbumin | XM_069580886.1 |
| Capra hircus | alpha-lactalbumin | X05149.1 |
| Moschus berezovskii | alpha-lactalbumin | XM_055394661.1 |
| Budorcas taxicolor | alpha-lactalbumin | XM_052641122.1 |
| Ovis aries | alpha-lactalbumin | NM_001009797.1 |
| Cervus canadensis | alpha-lactalbumin | XM_043446556.1 |
| Oryx dammah | alpha-lactalbumin | XM_040235500.1 |
| Oryx dammah | alpha-lactalbumin | XM_040235498.1 |
| Cervus elaphus | alpha-lactalbumin | XM_043878480.1 |
| Odocoileus virginianus texanus | alpha-lactalbumin | XM_020901385.1 |
| Capricornis sumatraensis | alpha-lactalbumin | XM_068971843.1 |
| Muntiacus reevesi | alpha-lactalbumin | XM_065932142.1 |
| Dama dama | alpha-lactalbumin | XM_061122769.1 |
| Bos taurus | alpha-lactalbumin | X63317.1 |
| Neophocaena asiaeorientalis asiaeorientalis | alpha-lactalbumin | XM_024736942.1 |
| Phocoena sinus | alpha-lactalbumin | XM_032646039.1 |
| Phocoena phocoena | alpha-lactalbumin | XM_065886974.1 |
| Pseudorca crassidens | alpha-lactalbumin | XM_067698650.1 |
| Monodon monoceros | alpha-lactalbumin | XM_029204514.1 |
| Globicephala melas | alpha-lactalbumin | XM_030835000.2 |
| Delphinus delphis | alpha-lactalbumin | XM_060025963.1 |
| Delphinapterus leucas | alpha-lactalbumin | XM_022572341.1 |
| Lagenorhynchus albirostris | alpha-lactalbumin | XM_060165326.1 |
| Tursiops truncatus | alpha-lactalbumin | XM_004324870.1 |
| Lagenorhynchus obliquidens | alpha-lactalbumin | XM_027108224.1 |
| Eschrichtius robustus | alpha-lactalbumin | XM_068561939.1 |
| Lipotes vexillifer | alpha-lactalbumin | XM_007452228.1 |
| Physeter macrocephalus | alpha-lactalbumin | XM_007107529.1 |
| Orcinus orca | alpha-lactalbumin | XM_004274395.1 |
| Eubalaena glacialis | alpha-lactalbumin | XM_061205971.1 |
| Balaenoptera musculus | alpha-lactalbumin | XM_036866566.1 |
| Balaenoptera ricei | alpha-lactalbumin | XM_059937352.1 |
| Kogia breviceps | alpha-lactalbumin | XM_067010616.1 |
| Balaenoptera acutorostrata | alpha-lactalbumin | XM_007179263.2 |
| Hippopotamus amphibius kiboko | alpha-lactalbumin | XM_057702754.1 |
| Mesoplodon densirostris | alpha-lactalbumin | XM_060114191.1 |
| synthetic construct | alpha-lactalbumin | KP096317.1 |
| Nyctereutes procyonoides | alpha-lactalbumin | XM_055337225.1 |
| Vulpes lagopus | alpha-lactalbumin | XM_041735717.1 |
| Vulpes vulpes | alpha-lactalbumin | XM_026000062.1 |
| Orycteropus afer afer | alpha-lactalbumin | XM_007937876.1 |
| Canis lupus dingo | alpha-lactalbumin | XM_025477270.2 |
| Canis lupus familiaris | lysozyme G | HG931825.1 |
| Manis pentadactyla | alpha-lactalbumin | XM_036890304.2 |
| Canis lupus familiaris | alpha-lactalbumin | NM_001003129.1 |
| Manis javanica | alpha-lactalbumin | XM_017646542.2 |
| Acinonyx jubatus | alpha-lactalbumin | XM_015065870.3 |
| Leopardus geoffroyi | alpha-lactalbumin | XM_045462931.1 |
| Felis catus | alpha-lactalbumin | XM_003988615.5 |
| Prionailurus viverrinus | alpha-lactalbumin | XM_047864861.1 |
| Castor canadensis | alpha-lactalbumin | XM_020188422.1 |
| Panthera onca | alpha-lactalbumin | XM_060657794.1 |
| Lynx canadensis | alpha-lactalbumin | XM_030322067.1 |
| Panthera leo | alpha-lactalbumin | XM_042948479.1 |
| Neofelis nebulosa | alpha-lactalbumin | XM_058742960.1 |
| Halichoerus grypus | alpha-lactalbumin | XM_036108932.1 |
| Hyaena hyaena | alpha-lactalbumin | XM_039221092.1 |
| Suricata suricatta | alpha-lactalbumin | XM_029954363.1 |
| Puma yagouaroundi | alpha-lactalbumin | XM_040488300.1 |
| Puma concolor | alpha-lactalbumin | XM_025930597.1 |
| Prionailurus bengalensis | alpha-lactalbumin | XM_043561749.1 |
| Saimiri boliviensis boliviensis | alpha-lactalbumin | XM_003927773.3 |
| Lynx rufus | alpha-lactalbumin | XM_047067297.1 |
| Ursus maritimus | alpha-lactalbumin | XM_008704516.2 |
| Ursus americanus | alpha-lactalbumin | XM_045801169.1 |
| Panthera pardus | alpha-lactalbumin | XM_019419199.2 |
| Neomonachus schauinslandi | alpha-lactalbumin | XM_021704870.1 |
| Leptonychotes weddellii | alpha-lactalbumin | NM_001290070.1 |
| Panthera tigris | alpha-lactalbumin | XM_007072954.3 |
| Meles meles | alpha-lactalbumin | XM_046013755.1 |
| Pan paniscus | alpha-lactalbumin | XM_003825800.5 |
| Pan paniscus | alpha-lactalbumin | XM_057299579.2 |
| Pan troglodytes | alpha-lactalbumin | XM_016924811.4 |
| Mirounga angustirostris | alpha-lactalbumin | XM_045868409.2 |
| Ursus arctos | alpha-lactalbumin | XM_026501566.4 |
| Panthera uncia | alpha-lactalbumin | XM_049627288.1 |
| Ailuropoda melanoleuca | alpha-lactalbumin | XM_002927206.4 |
| Mirounga leonina | alpha-lactalbumin | XM_035002042.1 |
| Pteronotus mesoamericanus | alpha-lactalbumin | XM_054590872.1 |
| Symphalangus syndactylus | alpha-lactalbumin | XM_063610654.1 |
| Nomascus leucogenys | alpha-lactalbumin | XM_003252212.3 |
| Homo sapiens | alpha-lactalbumin | NM_001384350.1 |
| Gorilla gorilla gorilla | alpha-lactalbumin | XM_004053050.4 |
| Homo sapiens | alpha-lactalbumin | NM_002289.3 |

In one aspect, the DNA constructs disclosed herein incorporate a gene that encodes casein. In a further aspect, casein is a family of related proteins that are typically found in phosphorylated forms. Caseins are hydrophobic and usually found in mammalian milk as components of micelles. Casein levels in different mammalian milks can vary from up to 80% in cows' milk and as low as 20% in human milk, although levels may vary based on environmental circumstances as well as genetics. Casein is particularly high in proline but contains all nine essential amino acids. Kappa-casein found in milk can be hydrolyzed into an insoluble peptide and water-soluble GMP.

In another aspect, caseins, including but not limited to, kappa-casein, may confer additional benefits such as, for example, anti-inflammatory properties and immune protection, as well as assisting efficient oxygen use by cells and electron transport. Thus, in one aspect, caseins are beneficial for energy and maintenance of cellular function and metabolism. In a still further aspect, casein may gel in the stomach, which enables casein to provide a slow, sustained release of amino acids.

In one aspect, the gene that encodes casein is isolated from domestic sheep. In a further aspect, the gene that encodes casein has SEQ ID NO. 4 or at least 70% homology thereto, at least 75% homology thereto, at least 80% homology thereto, at least 85% homology thereto, at least 90% homology thereto, at least 95% homology thereto, or at least 99% homology thereto.

Other sequences encoding casein or related or homologous genes can be identified in a database such as, for example, GenBank. In one aspect, the gene that encodes casein is isolated from Ovis aries and can be identified by the GI number NM_001009363.1 in the GenBank database. In another aspect, sequences useful herein include those with GI numbers listed in Table 4:

TABLE 4
Casein

| Source Organism | Sequence Description | GI Number |
|---|---|---|
| Ovis aries | alpha-s2-casein | NM_001009363.1 |
| Ovis canadensis | alpha-s2-casein | XM_069595034.1 |
| Ovis aries | alpha-s2-casein | GU169088.1 |
| Ovis aries | alpha-s2-casein | GU169085.1 |
| Ovis aries | alpha-s2-casein | GU169089.1 |
| Ovis aries | alpha-s2-casein | GU169086.1 |
| Ovis aries | alpha-s2-casein | GU169087.1 |
| Capra hircus | isolated mRNA | MN032075.1 |
| Capra hircus | isolated mRNA | MN219463.1 |
| Capra hircus | isolated mRNA | MN219447.1 |
| Capra hircus | isolated mRNA | MN032073.1 |
| Capra hircus | isolated mRNA | MN262129.1 |
| Capra hircus | isolated mRNA | MN219455.1 |
| Capra hircus | isolated mRNA | MN219446.1 |
| Capra hircus | isolated mRNA | MN219462.1 |
| Capra hircus | isolated mRNA | MN219448.1 |
| Capra hircus | isolated mRNA | MN262147.1 |
| Capra hircus | isolated mRNA | MN262144.1 |
| Capra hircus | alpha-s2-casein | NM_001285585.1 |
| Capra hircus | isolated mRNA | MN032080.1 |
| Capra hircus | isolated mRNA | MN032074.1 |
| Capra hircus | isolated mRNA | MN032077.1 |
| Capra hircus | isolated mRNA | MN262130.1 |
| Capra hircus | isolated mRNA | MN032079.1 |
| Capra hircus | isolated mRNA | MN262126.1 |
| Capra hircus | isolated mRNA | MN219457.1 |
| Capra hircus | isolated mRNA | MN262146.1 |
| Capra hircus | isolated mRNA | MN262128.1 |
| Capra hircus | isolated mRNA | MN219464.1 |
| Capra hircus | isolated mRNA | MN032076.1 |
| Capra hircus | alpha-s2-casein | S74171.1 |
| Capra hircus | isolated mRNA | MN219444.1 |
| Capra hircus | isolated mRNA | MN032071.1 |
| Capra hircus | isolated mRNA | MN219441.1 |
| Capra hircus | alpha-s2-casein | AJ289716.1 |
| Capra hircus | isolated mRNA | MN032069.1 |
| Capra hircus | isolated mRNA | MN262148.1 |
| Capra hircus | isolated mRNA | MN262131.1 |
| Capra hircus | isolated mRNA | MN032078.1 |
| Capra hircus | isolated mRNA | MN219443.1 |
| Capra hircus | isolated mRNA | MN219445.1 |
| Capra hircus | isolated mRNA | MN032072.1 |
| Capra hircus | isolated mRNA | MN262127.1 |
| Capra hircus | isolated mRNA | MN262149.1 |
| Capra hircus | isolated mRNA | MN219450.1 |
| Capra hircus | isolated mRNA | MN262145.1 |
| Capra hircus | alpha-s2-casein | XM_013964673.2 |
| Capra hircus | alpha-s2-casein | AJ289715.1 |
| Capra hircus | isolated mRNA | MN219451.1 |
| Capra hircus | alpha-s2-casein | AJ249995.1 |
| Budorcas taxicolor | alpha-s2-casein | XM_052641776.1 |
| Capra hircus | isolated mRNA | MN219458.1 |
| Capra hircus | isolated mRNA | MN032070.1 |
| Capricornis sumatraensis | isolated mRNA | XM_068974963.1 |
| Capra hircus | isolated mRNA | MN219442.1 |
| Oryx dammah | isolated mRNA | XM_040237324.1 |
| Moschus berezovskii | alpha-s2-casein | XM_055434376.1 |
| Capra hircus | alpha-s2-casein | XM_013964674.2 |
| Capra hircus | isolated mRNA | MN219461.1 |
| Capra hircus | isolated mRNA | MN219459.1 |
| Capra hircus | isolated mRNA | MN219460.1 |
| Capra hircus | isolated mRNA | MN219454.1 |
| Capra hircus | isolated mRNA | MN219456.1 |
| Capra hircus | alpha-s2-casein | XM_013964676.2 |
| Capra hircus | isolated mRNA | MN219453.1 |
| Capra hircus | isolated mRNA | MN219440.1 |
| Capra hircus | isolated mRNA | MN219439.1 |
| Capra hircus | alpha-s2-casein | XM_013964675.2 |
| Bubalus bubalis | alpha-s2-casein | NM_001290865.1 |
| Capra hircus | isolated mRNA | MN219452.1 |
| Capra hircus | isolated mRNA | MN219449.1 |
| Bubalus kerabau | alpha-s2-casein | XM_055587415.1 |
| Bubalus bubalis bubalis | alpha-s2-casein | KY399458.2 |
| Bubalus bubalis | alpha-s2-casein | XM_044945493.1 |
| Bubalus bubalis | alpha-s2-casein | FM865618.1 |
| Bubalus bubalis | alpha-s2-casein | DQ173244.1 |
| Bubalus bubalis | alpha-s2-casein | DQ133467.1 |
| Cervus canadensis | alpha-s2-casein | XM_043448107.1 |
| Cervus elaphus | alpha-s2-casein | XM_043906557.1 |
| Bos indicus | alpha-s2-casein | XM_019962871.1 |
| Bos taurus | alpha-s2-casein | XM_024993017.2 |
| Bos javanicus | alpha-s2-casein | XM_061420383.1 |
| Bos javanicus | alpha-s2-casein | XM_061420382.1 |
| Bos indicus x Bos taurus | alpha-s2-casein | XM_027545311.1 |
| Bubalus bubalis | alpha-s2-casein | JQ292811.1 |
| Bos mutus | alpha-s2-casein | XM_014480230.1 |
| Bos grunniens | alpha-s2-casein | MH378279.1 |
| Bos taurus | alpha-s2-casein | NM_174528.2 |
| Bison bison bison | alpha-s2-casein | XM_010852145.1 |
| Odocoileus virginianus texanus | alpha-s2-casein | XM_020873528.1 |
| Bos taurus | alpha-s2-casein | BC114773.1 |
| Bubalus bubalis | alpha-s2-casein | AJ005431.2 |
| Bubalus bubalis | alpha-s2-casein | FM865619.1 |
| Bubalus bubalis | alpha-s2-casein | KX896650.1 |
| Bubalus bubalis | alpha-s2-casein | XM_044945496.1 |
| Capra hircus | alpha-s2-casein | XM_013964678.2 |
| Bubalus bubalis | alpha-s2-casein | XM_044945489.1 |
| Dama dama | isolated mRNA | XM_061145571.1 |
| Bubalus bubalis | alpha-s2-casein | XM_044945495.1 |
| Bubalus bubalis | alpha-s2-casein | XM_044945494.2 |

In an aspect, the protein-producing DNA constructs can have SEQ ID NO. 5 or at least 70% homology thereto, at least 80% homology thereto, at least 90% homology thereto, at least 95% homology thereto, or at least 99% homology thereto. In some aspects, the protein-producing DNA constructs can also include genes encoding one or more salivary enzymes such as, for example, one or more cathepsins, serine proteases, peptidases, or any combination thereof.

### Additional DNA Constructs

In some aspects, in addition to lysates and extracts from the DNA constructs expressing complete proteins described above, lysates and extracts from one or more additional DNA constructs may be added to the disclosed compositions in order to supplement the nutrition provided by the proteins. In an aspect, these additional DNA constructs may provide one or more vitamins or antioxidants such as, for example, lycopene, or may provide a flavorant or sweetener such as, for example, steviol glycosides, an organic electrolyte, or another beneficial compound. These additional DNA constructs are described in more detail below:

In one aspect, the additional DNA constructs disclosed herein incorporate a gene that encodes lycopene cyclase. In a further aspect, lycopene cyclase (may be an α-, β-, γ-, or ϵ-lycopene cyclase) is an enzyme that catalyzes the conversion of lycopene to β-carotene or another provitamin A carotenoid. Lycopene cyclase requires an NAD(P)H cofactor, or, in some cases, a reduced FAD cofactor.

In one aspect, the gene that encodes lycopene cyclase is isolated from corn. In a further aspect, the gene that encodes lycopene cyclase has SEQ ID NO. 6 or at least 70% homology thereto, at least 75% homology thereto, at least 80% homology thereto, at least 85% homology thereto, at least 90% homology thereto, at least 95% homology thereto, or at least 99% homology thereto.

Other sequences encoding lycopene cyclase or related or homologous genes can be identified in a database such as, for example, GenBank. In one aspect, the gene that encodes lycopene cyclase is isolated from Zea mays and can be identified by the GI number EU924262.1 in the GenBank database. In another aspect, sequences useful herein include those with GI numbers listed in Table 5:

TABLE 5
Lycopene Cyclase

| Source Organism | Sequence Description | GI Number |
|---|---|---|
| Zea mays | lycopene-epsilon cyclase | EU924262.1 |
| Zea mays | uncharacterized | NM_001153368.1 |
| Miscanthus floridulus | lycopene-epsilon cyclase | XM_066455949.1 |
| Miscanthus floridulus | lycopene-epsilon cyclase | XM_066455944.1 |
| Sorghum bicolor | lycopene-epsilon cyclase | XM_002455793.2 |
| Miscanthus floridulus | lycopene-epsilon cyclase | XM_066450343.1 |
| Setaria viridis | lycopene-epsilon cyclase | XM_034742532.1 |
| Setaria italica | lycopene-epsilon cyclase | XM_004969360.3 |
| Panicum virgatum | lycopene-epsilon cyclase | XM_039954585.1 |
| Panicum hallii | lycopene-epsilon cyclase | XM_025962889.1 |
| Zea mays | uncharacterized | BT063754.1 |
| Panicum virgatum | lycopene-epsilon cyclase | XM_039992031.1 |
| Phragmites australis | lycopene-epsilon cyclase | XM_062347166.1 |
| Brachypodium distachyon | lycopene-epsilon cyclase | XM_003569209.4 |
| Lolium rigidum | lycopene-epsilon cyclase | XM_047196978.1 |
| Hordeum vulgare subsp. vulgare | lycopene-epsilon cyclase | XM_045120293.1 |
| Aegilops tauschii subsp. strangulata | lycopene-epsilon cyclase | XM_020297682.3 |
| Hordeum vulgare subsp. vulgare | uncharacterized | AK371513.1 |
| Oryza glaberrima | lycopene-epsilon cyclase | XM_052309458.1 |
| Oryza brachyantha | lycopene-epsilon cyclase | XM_006644249.3 |
| Triticum aestivum | lycopene-epsilon cyclase | XM_044492993.1 |
| Triticum dicoccoides | lycopene-epsilon cyclase | XM_037557147.1 |
| Triticum urartu | lycopene-epsilon cyclase | XM_048707511.1 |
| Lolium perenne | lycopene-epsilon cyclase | XM_051368750.1 |
| Oryza sativa Japonica Group | uncharacterized | AK072015.1 |
| Oryza sativa Japonica Group | uncharacterized | AK066182.1 |
| Oryza sativa Japonica Group | lycopene-epsilon cyclase | XM_015766712.3 |
| Ricinus communis | lycopene-epsilon cyclase | XM_015716320.3 |
| Lycium ruthenicum | lycopene-epsilon cyclase | KF957711.1 |
| Carica papaya | lycopene-epsilon cyclase | XM_022046323.1 |
| Lycium barbarum | lycopene-epsilon cyclase | XM_060353965.1 |
| Lycium barbarum | lycopene-epsilon cyclase | KF957687.1 |
| Lycium chinense | lycopene-epsilon cyclase | KF768738.2 |
| Lycium ferocissimum | lycopene-epsilon cyclase | XM_059420702.1 |
| Ricinus communis | lycopene-epsilon cyclase | XR_003078295.2 |
| Mercurialis annua | lycopene-epsilon cyclase | XM_050361483.2 |
| Macadamia integrifolia | lycopene-epsilon cyclase | XM_042627017.1 |
| Elaeis guineensis | lycopene-epsilon cyclase | XM_010940441.3 |
| Daucus carota | lycopene-epsilon cyclase | OQ867884.1 |
| Daucus carota | lycopene-epsilon cyclase | OQ867885.1 |
| Daucus carota | lycopene-epsilon cyclase | OQ867883.1 |
| Chelidonium majus | lycopene-epsilon cyclase | MW307339.1 |
| Carica papaya | lycopene-epsilon cyclase | XM_022046324.1 |
| Daucus carota | lycopene-epsilon cyclase | ON455109.1 |
| Daucus carota subsp. sativus | lycopene-epsilon cyclase | NM_001329163.1 |
| Daucus carota | lycopene-epsilon cyclase | OQ867887.1 |
| Daucus carota subsp. sativus | lycopene-epsilon cyclase | DQ192192.1 |
| Daucus carota | lycopene-epsilon cyclase | OQ867886.1 |
| Abelmoschus esculentus | lycopene-epsilon cyclase | KX257999.1 |
| Amborella trichopoda | lycopene-epsilon cyclase | XM_011628959.2 |
| Phoenix dactylifera | lycopene-epsilon cyclase | XM_008814731.4 |
| Pisum sativum | lycopene-epsilon cyclase | XM_051062965.1 |
| Jatropha curcas | lycopene-epsilon cyclase | XM_012221559.2 |
| Nelumbo nucifera | lycopene-epsilon cyclase | XM_010263735.2 |
| Trifolium pratense | lycopene-epsilon cyclase | XM_045931564.1 |
| Medicago truncatula | lycopene-epsilon cyclase | XM_003595195.4 |
| Durio zibethinus | lycopene-epsilon cyclase | XM_022896008.1 |
| Buddleja davidii | carotene epsilon-monooxygenase | MT460463.1 |
| Solanum dulcamara | lycopene-epsilon cyclase | XM_055945632.1 |
| Hevea brasiliensis | lycopene-epsilon cyclase | XM_021782260.2 |
| Nelumbo nucifera | lycopene-epsilon cyclase | XM_010263734.2 |
| Gossypium arboreum | lycopene-epsilon cyclase | XM_017787896.2 |
| Amborella trichopoda | lycopene-epsilon cyclase | XM_020673915.1 |
| Amborella trichopoda | lycopene-epsilon cyclase | XM_020673917.1 |
| Gossypium arboreum | lycopene-epsilon cyclase | XM_053028437.1 |
| Salvia hispanica | lycopene-epsilon cyclase | XM_048096110.1 |
| Amborella trichopoda | lycopene-epsilon cyclase | XM_020673916.1 |
| Nicotiana tabacum | lycopene-epsilon cyclase | XM_016658602.1 |
| Heracleum moellendorffii | lycopene-epsilon cyclase | OM732408.1 |
| Narcissus tazetta subsp. chinensis | lycopene-epsilon cyclase | JQ282902.1 |
| Nicotiana attenuata | lycopene-epsilon cyclase | XM_019384536.1 |
| Sesamum indicum | lycopene-epsilon cyclase | XM_020693771.1 |
| Sesamum indicum | lycopene-epsilon cyclase | XM_011079724.2 |
| Hordeum chilense | lycopene-epsilon cyclase | KC962403.1 |
| Sesamum indicum | lycopene-epsilon cyclase | XM_020693772.1 |
| Nicotiana sylvestris | lycopene-epsilon cyclase | XM_009781191.1 |
| Diospyros lotus | lycopene-epsilon cyclase | XM_052344251.1 |
| Camellia sinensis | lycopene-epsilon cyclase | XM_028218415.1 |
| Hordeum chilense | lycopene-epsilon cyclase | KC962404.1 |
| Musa acuminata AAA Group | lycopene-epsilon cyclase | MK616524.1 |
| Musa acuminata AAA Group | lycopene-epsilon cyclase | XM_009406869.3 |
| Musa acuminata AAA Group | lycopene-epsilon cyclase | XM_065091989.1 |
| Musa acuminata AAA Group | lycopene-epsilon cyclase | XM_065091991.1 |
| Musa acuminata AAA Group | lycopene-epsilon cyclase | XM_065091992.1 |
| Gossypium hirsutum | lycopene-epsilon cyclase | XM_016870694.2 |
| Gossypium hirsutum | lycopene-epsilon cyclase | XM_016870691.2 |
| Euphorbia lathyris | lycopene-epsilon cyclase | XM_065992984.1 |
| Ananas comosus | lycopene-epsilon cyclase | XM_020250148.1 |
| Musa acuminata AAA Group | lycopene-epsilon cyclase | XM_065111223.1 |
| Nicotiana tabacum | lycopene-epsilon cyclase | XM_016611548.1 |
| Salvia splendens | lycopene-epsilon cyclase | XM_042206364.1 |
| Narcissus tazetta subsp. chinensis | lycopene-epsilon cyclase | JQ282903.1 |
| Solanum stenotomum | lycopene-epsilon cyclase | XM_049542490.1 |
| Ziziphus jujuba | lycopene-epsilon cyclase | XM_060818958.1 |
| Chenopodium quinoa | lycopene-epsilon cyclase | XR_002503549.1 |
| Chenopodium quinoa | lycopene-epsilon cyclase | XM_021859378.1 |
| Chenopodium quinoa | lycopene-epsilon cyclase | XM_021859377.1 |
| Actinidia eriantha | lycopene-epsilon cyclase | XM_057644032.1 |
| Hibiscus syriacus | lycopene-epsilon cyclase | XM_039208639.1 |
| Mercurialis annua | lycopene-epsilon cyclase | XR_007638344.2 |

In one aspect, the DNA constructs disclosed herein incorporate a gene that encodes β-carotene hydroxylase. In a further aspect, β-carotene hydroxylase is an enzyme that catalyzes the conversion of β-carotene to other carotenoids such as, for example, zeaxanthin, cryptoxanthin, or a combination thereof, as well as the reverse reactions back to β-carotene. \beta-carotene hydroxylase requires an NADH cofactor as well as ferredoxin and iron(II) ions.

In one aspect, the gene that encodes-carotene hydroxylase is isolated from a Parasynechococcus or Synechococcus species. In a further aspect, the gene that encodes β-carotene hydroxylase has SEQ ID NO. 7 or at least 70% homology thereto, at least 75% homology thereto, at least 80% homology thereto, at least 85% homology thereto, at least 90% homology thereto, at least 95% homology thereto, or at least 99% homology thereto.

Other sequences encoding β-carotene hydroxylase or related or homologous genes can be identified in a database such as, for example, GenBank. In one aspect, the gene that encodes β-carotene hydroxylase is isolated from Parasynechococcus marenigrum WH 8102 and can be identified by the GI number BX569689.1 in the GenBank database. In another aspect, sequences useful herein include those with GI numbers listed in Table 6:

TABLE 6
β-Carotene Hydroxylase

| | Sequence | |
|---|---|---|
| Source Organism | Description | GI Number |
| Parasynechococcus marenigrum WH 8102 | genomic DNA | BX569689.1 |
| Synechococcus sp. A15-24 | genomic DNA | CP047960.1 |
| Synechococcus sp. PROS-U-1 | genomic DNA | CP047951.1 |
| Synechococcus sp. A15-28 | genomic DNA | CP047931.1 |
| Synechococcus sp. WH 8109 | genomic DNA | CP006882.1 |
| Synechococcus sp. KORDI-52 | genomic DNA | CP006271.1 |
| Synechococcus sp. MIT S9220 | genomic DNA | CP047958.1 |
| Synechococcus sp. M16.1 | genomic DNA | CP047954.1 |
| Synechococcus sp. TAK9802 | genomic DNA | CP047937.1 |
| Synechococcus sp. RS9902 | genomic DNA | CP047949.1 |
| Synechococcus sp. RS9907 | genomic DNA | CP047944.1 |
| Synechococcus sp. BIOS-U3-1 | genomic DNA | CP047936.1 |
| Synechococcus sp. A15-62 | genomic DNA | CP047950.1 |
| Synechococcus sp. A15-44 | genomic DNA | CP047938.1 |
| Prochlorococcus sp. MIT 1300 | genomic DNA | CP139302.1 |
| Synechococcus sp. PROS-9-1 | genomic DNA | CP047961.1 |
| Synechococcus sp. M16CYN | genomic DNA | AP029048.1 |
| Synechococcus sp. A15-127 | genomic DNA | CP047948.1 |
| Synechococcus sp. CC9902 | genomic DNA | CP000097.1 |
| Synechococcus sp. BIOS-E4-1 | genomic DNA | CP047935.1 |
| Prochlorococcus marinus str. MIT 1013 | genomic DNA | CP114778.1 |
| Synechococcus sp. SYN20 | genomic DNA | CP047959.1 |
| Synechococcus sp. MVIR-18-1 | genomic DNA | CP047942.1 |
| Prochlorococcus sp. MIT 1223 | genomic DNA | CP139303.1 |
| Synechococcus sp. CC9605 | genomic DNA | CP000110.1 |
| Prochlorococcus sp. MIT 1307 | genomic DNA | CP139301.1 |
| Synechococcus sp. KORDI-49 | genomic DNA | CP006270.1 |
| Prochlorococcus marinus str. MIT 0912 | genomic DNA | CP114783.1 |
| Synechococcus sp. CC9311 | genomic DNA | CP000435.1 |
| Paulinella chromatophora | genomic DNA | NC_011087.1 |
| Synechococcus sp. WH 8020 | genomic DNA | CP011941.1 |
| Prochlorococcus marinus str. MIT 0913 | genomic DNA | CP114782.1 |
| Prochlorococcus marinus subsp. marinus | genomic DNA | AE017126.1 |
| str. CCMP1375 | | |
| Prochlorococcus marinus str. MIT 0915 | genomic DNA | CP114781.1 |
| Synechococcus sp. KORDI-100 | genomic DNA | CP006269.1 |
| Prochlorococcus sp. MIT 0801 | genomic DNA | CP007754.1 |
| Prochlorococcus marinus str. MIT 0917 | genomic DNA | CP114784.1 |
| Prochlorococcus marinus str. NATL1A | genomic DNA | CP000553.1 |
| Prochlorococcus marinus str. NATL2A | genomic DNA | CP000095.2 |
| Prochlorococcus sp. RS01 | genomic DNA | CP018345.1 |
| Prochlorococcus sp. RS50 | genomic DNA | CP018344.1 |
| Prochlorococcus sp. RS04 | genomic DNA | CP018346.1 |
| Prochlorococcus marinus str. MIT 9303 | genomic DNA | CP000554.1 |
| Prochlorococcus marinus str. AS9601 | genomic DNA | CP000551.1 |
| Prochlorococcus marinus str. MIT 0919 | genomic DNA | CP114779.1 |
| Synechococcus sp. ROS8604 | genomic DNA | CP047946.1 |
| Prochlorococcus marinus str. MIT 1214 | genomic DNA | CP114777.1 |
| Synechococcus sp. A18-25c | genomic DNA | CP047957.1 |
| Prochlorococcus marinus str. MIT 9211 | genomic DNA | CP000878.1 |
| Prochlorococcus sp. AG-321-021 | genomic DNA | MH327327.1 |
| Paulinella longichromatophora | genomic DNA | MG264610.1 |
| Prochlorococcus marinus str. MIT 9301 | genomic DNA | CP000576.1 |
| Prochlorococcus marinus str. MIT 9515 | genomic DNA | CP000552.1 |
| Synechococcus sp. MEDNS5 | genomic DNA | CP047952.1 |
| Synechococcus sp. A15-60 | genomic DNA | CP047933.1 |
| Synechococcus sp. HK01-R | genomic DNA | CP059059.1 |
| Prochlorococcus marinus str. MIT 9313 | genomic DNA | BX548175.1 |
| Prochlorococcus sp. MIT 1314 | genomic DNA | CP139300.1 |
| Prochlorococcus marinus str. MIT 9312 | genomic DNA | CP000111.1 |
| Prochlorococcus marinus subsp. pastoris | genomic DNA | BX548174.1 |
| str. CCMP1986 | | |
| Paulinella micropora | genomic DNA | KX897545.1 |
| Paulinella micropora | genomic DNA | KY124271.1 |
| Synechococcus sp. PROS-7-1 | genomic DNA | CP047945.1 |
| Prochlorococcus sp. MIT 0604 | genomic DNA | CP007753.1 |
| Synechococcus sp. WH 8101 | genomic DNA | CP047932.1 |
| Synechococcus sp. WH 8101 | genomic DNA | CP035914.1 |
| Synechococcus sp. BMK-MC-1 | genomic DNA | CP047939.1 |
| Paulinella micropora | genomic DNA | NC_039737.1 |
| Prochlorococcus marinus str. MIT 9215 | genomic DNA | CP000825.1 |
| Synechococcus sp. WH 7803 | genomic DNA | CT971583.1 |
| Synechococcus sp. NOUM97013 | genomic DNA | CP047941.1 |
| Cyanobium usitatum str. Tous | genomic DNA | OY986431.1 |
| Prochlorococcus marinus str. MIT 0918 | genomic DNA | CP114780.1 |
| Synechococcus sp. RS9909 | genomic DNA | CP047943.1 |
| Synechococcus sp. CB0101 | genomic DNA | CP039373.1 |
| Synechococcus sp. RCC307 | genomic DNA | CT978603.1 |
| Synechococcus sp. Minos11 | genomic DNA | CP047953.1 |
| Synechococcus sp. LA31 | genomic DNA | CP075523.1 |
| Synechococcus sp. CBW1108 | genomic DNA | CP060395.1 |
| Synechococcus sp. A10-1-5-1 | genomic DNA | CP096032.1 |
| Synechococcus sp. LTW-R | genomic DNA | CP059060.1 |
| Synechococcus sp. CBW1107 | genomic DNA | OY986430.1 |
| Cyanobium sp. NS01 | genomic DNA | CP047940.1 |
| Arthrospira platensis YZ | genomic DNA | CP013008.1 |
| Arthrospira sp. PCC 9108 | genomic DNA | CP066886.2 |
| Limnospira fusiformis KN01 | genomic DNA | CP091467.1 |
| Limnospira fusiformis SAG 85.79 | genomic DNA | CP051185.1 |
| Limnospira indica PCC 8005 | genomic DNA | FO818640.1 |
| Arthrospira platensis C1 | genomic DNA | CP019998.1 |
| Limnospira indica BM01 | genomic DNA | CP060212.1 |
| Halomicronema hongdechloris C2206 | genomic DNA | CP021983.2 |
| Cyanobium sp. NIES-981 | genomic DNA | LT578417.1 |
| Galdieria partita | genomic DNA | AP025529.1 |
| Arthrospira platensis NIES-39 | genomic DNA | AP026945.2 |
| Cyanobium sp. M30B3 | genomic DNA | CP073761.1 |
| Synechococcus sp. CCFWC 502 | genomic DNA | CP114977.1 |
| Synechococcus sp. RSCCF101 | genomic DNA | CP035632.1 |
| Galdieria sulphuraria | genomic DNA | OP616815.1 |
| Galdieria sp. | genomic DNA | MZ681961.1 |
| Synechococcus sp. NB0720_010 | genomic DNA | CP090898.1 |

In one aspect, the DNA constructs disclosed herein incorporate a gene that encodes 1-deoxy-D-xylulose-5-phosphate synthase (DXS), also referred to herein as steviol synthase. In a further aspect, DXS is a non-mevalonate enzyme that requires pyruvate and D-glyceraldehyde 3-phosphate as starting materials, as well as thiamine diphosphate as a cofactor, to produce 1-deoxy-D-xylulose-5-phosphate. Carbon dioxide is produced as a byproduct of this reaction. Although this enzyme is required for terpenoid biosynthesis, depending on the chemical environment, other substrates can also be modified with this enzyme.

In one aspect, the gene that encodes DXS is isolated from a Stevia species. In a further aspect, the gene that encodes DXS has SEQ ID NO. 8 or at least 70% homology thereto, at least 75% homology thereto, at least 80% homology thereto, at least 85% homology thereto, at least 90% homology thereto, at least 95% homology thereto, or at least 99% homology thereto.

Other sequences encoding DXS or related or homologous genes can be identified in a database such as, for example, GenBank. In one aspect, the gene that encodes DXS is isolated from Stevia rebaudiana and can be identified by the GI number FJ214107.1 in the GenBank database. In another aspect, sequences useful herein include those with GI numbers listed in Table 7:

TABLE 7
1-Deoxy-D-Xylulose-5-Phosphate Synthase

| | Sequence | |
|---|---|---|
| Source Organism | Description | GI Number |
| Stevia rebaudiana | DXS | FJ214107.1 |
| Stevia rebaudiana | DXS | AJ429232.2 |
| Stevia rebaudiana | DXS | KT276232.1 |
| Helianthus annuus | DXS | XM_022142075.2 |
| Erigeron canadensis | DXS | XM_043751304.1 |
| Chrysanthemum x morifolium | DXS | AB205044.1 |
| Taraxacum kok-saghyz | DXS | KT899414.1 |
| Lactuca sativa | DXS | XM_023911233.3 |
| Cynara cardunculus var. scolymus | DXS | XM_025119573.1 |
| Matricaria chamomilla | DXS | PQ458721.1 |
| Cornus florida | DXS | XM_059787233.1 |
| Actinidia eriantha | DXS | XM_057639887.1 |
| Manihot esculenta | DXS | XM_021774212.2 |
| Camellia sinensis | DXS | MH925308.1 |
| Camellia sinensis | DXS | XM_028246899.1 |
| Hevea brasiliensis | DXS | DQ473433.1 |
| Hevea brasiliensis | DXS | NM_001422026.1 |
| Hevea brasiliensis | DXS | AB294699.1 |
| Ricinus communis | DXS | XM_002532338.4 |
| Actinidia eriantha | DXS | XM_057635989.1 |
| Camellia sinensis | DXS | XM_028246900.1 |
| Eucommia ulmoides | DXS | JX458818.2 |
| Jatropha curcas | DXS | XM_012226308.3 |
| Erythranthe guttata | DXS | XM_012989649.1 |
| Diospyros lotus | DXS | XM_052342155.1 |
| Hevea brasiliensis | DXS | XM_058128504.1 |
| Hevea brasiliensis | DXS | XM_021781018.2 |
| Nicotiana tomentosiformis | DXS | XM_009592196.3 |
| Nigella sativa | DXS | MN548762.1 |
| Camellia sinensis | DXS | XM_028226722.1 |
| Juglans microcarpa x Juglans regia | DXS | XM_041160582.1 |
| Nicotiana attenuata | DXS | XM_019406517.1 |
| Manihot esculenta | DXS | XM_021776767.2 |
| Osmanthus fragrans | DXS | KX400842.1 |
| Manihot esculenta | DXS | XM_021776766.2 |
| Diospyros lotus | DXS | XM_052342154.1 |
| Bixa orellana | DXS | MW885531.1 |
| Bixa orellana | DXS | KT358984.1 |
| Alnus glutinosa | DXS | XM_062289528.1 |
| Malus sylvestris | DXS | XM_050270722.1 |
| Malus domestica | DXS | XM_008340252.3 |
| Juglans regia | DXS | XM_018954512.2 |
| Solanum dulcamara | DXS | XM_055973042.1 |
| Nicotiana tabacum | DXS | XM_016592817.1 |
| Nicotiana sylvestris | DXS | XM_009769974.1 |
| Mercurialis annua | DXS | XM_050350211.2 |
| Nicotiana tabacum | DXS | NM_001325159.1 |
| Rhododendron vialii | DXS | XM_058332015.1 |
| Gentiana rigescens | DXS | KM974886.1 |
| Corylus avellana | DXS | XM_059582267.1 |
| Solanum habrochaites | DXS | AY687353.1 |
| Carya illinoinensis | DXS | XM_043133572.1 |
| Pyrus x bretschneideri | DXS | XM_009366530.3 |
| Euphorbia lathyris | DXS | XM_066018529.1 |
| Pyrus communis | DXS | XM_068459907.1 |
| Pistacia vera | DXS | XM_031429295.1 |
| Ipomoea nil | DXS | XM_019327463.1 |
| Ipomoea nil | DXS | XM_019327462.1 |
| Capsicum annuum | DXS | XM_047400612.1 |
| Trachyspermum ammi | DXS | MG762014.1 |
| Pyrus x bretschneideri | DXS | XM_009365960.3 |
| Pistacia vera | DXS | XM_031429296.1 |
| Crataegus pinnatifida f. major | DXS | KR704420.1 |
| Crataegus pinnatifida f. major | DXS | KR704421.1 |
| Adonis aestivalis var. palaestina | DXS | EF043284.1 |
| Juglans microcarpa x Juglans regia | DXS | XM_041154065.1 |
| Solanum verrucosum | DXS | XM_049516272.1 |
| Solanum stenotomum | DXS | XM_049522951.1 |
| Daucus carota subsp. sativus | DXS | XM_017392124.2 |
| Solanum pennellii | DXS | XM_015203416.2 |
| Solanum lycopersicum | DXS | NM_001345870.1 |
| Ipomoea triloba | DXS | XM_031239661.1 |
| Ipomoea triloba | DXS | XM_031239662.1 |
| Pyrus communis | DXS | XM_068474221.1 |
| Populus nigra | DXS | XM_062092642.1 |
| Lycium barbarum | DXS | XM_060328891.1 |
| Solanum tuberosum | DXS | XM_006353091.2 |
| Populus euphratica | DXS | XM_011012876.1 |
| Malus domestica | DXS | XM_008353503.3 |
| Olea europaea var. sylvestris | DXS | XM_023038065.1 |
| Populus alba | DXS | XM_035034696.1 |
| Cucurbita maxima | DXS | XM_023129252.1 |
| Lycium ferocissimum | DXS | XM_059451405.1 |
| Lycium barbarum | DXS | KF957679.1 |
| Tripterygium wilfordii | DXS | KM879186.1 |
| Argentina anserina | DXS | XM_050515675.1 |
| Populus trichocarpa | DXS | XM_006380518.3 |
| Lycium ruthenicum | DXS | KF957703.1 |
| Catharanthus roseus | DXS | AJ011840.2 |
| Malus sylvestris | DXS | XM_050297854.1 |
| Trifolium pratense | DXS | XM_045973912.1 |
| Coffea eugenioides | DXS | XM_027322583.1 |
| Fragaria vesca subsp. vesca | DXS | XM_004289469.2 |
| Tripterygium wilfordii | DXS | XM_038863002.1 |
| Prunus avium | DXS | XM_021946477.1 |
| Tripterygium wilfordii | DXS | XM_038846259.1 |
| Prunus avium | DXS | XM_021946478.1 |
| Prunus dulcis | DXS | XM_034365770.1 |
| Olea europaea var. sylvestris | DXS | XM_022991093.1 |
| Impatiens glandulifera | DXS | XM_047454994.1 |

In an aspect, the constructs encoding antioxidant related genes and/or steviol production genes can have SEQ ID NO. 9 or SEQ ID NO. 10 or at least 70% homology thereto, at least 80% homology thereto, at least 90% homology thereto, at least 95% homology thereto, or at least 99% homology thereto.

In one aspect, the additional DNA constructs disclosed herein are useful for producing organic electrolytes. As such, in one aspect, the additional DNA constructs can incorporate a gene that expresses hydrogenase. Hydrogenase is a protein that catalyzes the reversible oxidation of molecular hydrogen. Numerous hydrogenases are recognized including [NiFe] hydrogenases, [NiFeSe] hydrogenases, [FeFe] hydrogenases, and [Fe]-only hydrogenases, where the chemical symbols in brackets indicate the metal ions at the catalytic centers of the protein.

In one aspect, the gene that encodes hydrogenase is isolated from a bacterium. In a further aspect, the gene that encodes hydrogenase has SEQ ID NO. 11 or at least 70% homology thereto, at least 75% homology thereto, at least 80% homology thereto, at least 85% homology thereto, at least 90% homology thereto, at least 95% homology thereto, or at least 99% homology thereto.

Other sequences encoding hydrogenase or related or homologous genes can be identified in a database such as, for example, GenBank. In one aspect, the gene that encodes chromate uranium reductase is isolated from Acidithiobacillus thiooxidans and can be identified by the GI number CP045571.1 in the GenBank database. In another aspect, sequences useful herein include those with GI numbers listed in Table 8:

TABLE 8
Hydrogenase

| | Sequence | |
|---|---|---|
| Source Organism | Description | GI Number |
| Acidithiobacillus thiooxidans ATCC 19377 | Genomic DNA | CP045571.1 |
| Acidithiobacillus sp. AMEEHan | Genomic DNA | CP118747.1 |
| Acidithiobacillus sp. ‘AMD consortium’ | Genomic DNA | CP044411.1 |
| Acidithiobacillus ferridurans | Genomic DNA | AP018795.1 |
| Acidithiobacillus ferrivorans | Genomic DNA | CP059488.1 |
| Acidithiobacillus ferrivorans | Genomic DNA | LT841305.1 |
| Acidithiobacillus ferrivorans SS3 | Genomic DNA | CP002985.1 |
| Acidithiobacillus ferriphilus | Genomic DNA | CP116458.1 |
| Acidithiobacillus sp. YTS05 | Genomic DNA | CP094359.1 |
| Acidithiobacillus ferrooxidans | Genomic DNA | CP084172.1 |
| Acidithiobacillus ferrooxidans | Genomic DNA | AP025160.1 |
| Acidithiobacillus ferrooxidans | Genomic DNA | CP082238.1 |
| Acidithiobacillus ferrooxidans | Genomic DNA | CP040511.1 |
| Acidithiobacillus ferrooxidans ATCC 23270 | Genomic DNA | CP001219.1 |
| Acidithiobacillus ferrooxidans ATCC 53993 | Genomic DNA | CP001132.1 |
| Sulfuriferula plumbiphila | Genomic DNA | AP021884.1 |
| Acidithiobacillus caldus | Genomic DNA | CP133598.1 |
| Acidithiobacillus caldus ATCC 51756 | Genomic DNA | CP005986.1 |
| Acidithiobacillus caldus SM-1 | Genomic DNA | CP002573.1 |
| Acidithiobacillus caldus | Genomic DNA | CP043926.1 |
| Acidithiobacillus caldus | Genomic DNA | CP026328.2 |
| Variovorax sp. PBL-H6 | Genomic DNA | LR594659.1 |
| Cupriavidus sp. KK10 | Genomic DNA | CP073678.1 |
| Sulfurimicrobium lacus | Genomic DNA | AP022853.1 |
| Cupriavidus necator N-1 | Genomic DNA | CP002879.1 |
| Cupriavidus sp. WKF15 | Genomic DNA | CP119573.1 |
| Polaromonas sp. P1-6 | Genomic DNA | CP087966.1 |
| Polaromonas sp. P1(28)-13 | Genomic DNA | CP087963.1 |
| Polaromonas sp. P2-4 | Genomic DNA | CP087965.1 |
| Variovorax paradoxus B4 | Genomic DNA | CP003912.1 |
| Gallionella capsiferriformans ES-2 | Genomic DNA | CP002159.1 |
| Polaromonas sp. P1(28)-8 | Genomic DNA | CP087964.1 |
| uncultured bacterium gwa2_scaffold_14 | CRISPR-Cas scaffold | KU516114.1 |
| Cupriavidus necator | Plasmid DNA | CP068436.1 |
| Burkholderia sp. HB1 | Genomic DNA | CP012193.1 |
| Burkholderiales bacterium GJ-E10 | Genomic DNA | AP014683.1 |
| uncultured bacterium | Genomic DNA | KX576128.1 |
| Denitratisoma oestradiolicum | Genomic DNA | LR778301.1 |
| Paraburkholderia dioscoreae | Genomic DNA | LR699554.1 |
| Cupriavidus necator H16 | Genomic DNA | CP039287.1 |
| Cupriavidus necator | Genomic DNA | CP129213.1 |
| Paraburkholderia terrae | Genomic DNA | AP025259.1 |
| Paraburkholderia xenovorans LB400 | Genomic DNA | CP008761.1 |
| Paraburkholderia terrae | Genomic DNA | AP024958.1 |
| Ralstonia pickettii DTP0602 | Genomic DNA | CP006667.1 |
| Cupriavidus necator | Genomic DNA | CP066018.1 |
| Cupriavidus necator H16 | Genomic DNA | AM260479.1 |
| Paraburkholderia xenovorans LB400 | Genomic DNA | CP000272.1 |
| Paraburkholderia aromaticivorans | Genomic DNA | CP051514.1 |
| Cupriavidus oxalaticus | Genomic DNA | CP038636.1 |
| Paucibacter sp. S2-9 | Genomic DNA | CP116346.1 |
| Paraburkholderia terricola | Genomic DNA | CP084255.1 |
| Paraburkholderia terricola | Genomic DNA | CP024941.1 |
| Dechloromonas sp. A34 | Genomic DNA | CP102486.1 |
| Polaromonas sp. JS666 | Genomic DNA | CP000316.1 |
| Paraburkholderia aromaticivorans | Genomic DNA | CP022991.1 |
| Paraburkholderia pallida | Genomic DNA | CP038152.1 |
| Variovorax sp. PBL-E5 | Plasmid DNA | LR594673.1 |
| Rhodoferax ferrireducens | Genomic DNA | CP138198.1 |
| Azoarcus sp. KH32C | Genomic DNA | AP012304.1 |
| Sideroxyarcus emersonii | Genomic DNA | AP023423.1 |
| Sulfuritalea hydrogenivorans sk43H | Genomic DNA | AP012547.1 |
| Paraburkholderia xenovorans LB400 | Genomic DNA | CP008762.1 |
| Paraburkholderia xenovorans LB400 | Genomic DNA | CP000271.1 |
| Rhodoferax ferrireducens T118 | Genomic DNA | CP000267.1 |
| Ralstonia syzygii R24 | Genomic DNA | CP115944.1 |
| Ralstonia syzygii R24 | Genomic DNA | FR854086.1 |
| Sideroxydans lithotrophicus ES-1 | Genomic DNA | CP001965.1 |
| Cupriavidus metallidurans | Plasmid DNA | CP046333.1 |
| Paraburkholderia sp. PGU19 | Genomic DNA | AP023182.1 |
| Cupriavidus metallidurans CH34 | Genomic DNA | CP000353.2 |
| Candidatus Desulfobacillus denitrificans | Genomic DNA | AP021857.1 |
| Paraburkholderia dokdonensis | Genomic DNA | CP029641.1 |
| Burkholderiaceae bacterium | Genomic DNA | CP126124.1 |
| Rugosibacter aromaticivorans | Genomic DNA | CP010554.1 |
| Paraburkholderia hospita | Genomic DNA | CP024939.1 |
| Acidiferrobacter sp. SPIII_3 | Genomic DNA | CP027663.1 |
| Paraburkholderia hospita | Genomic DNA | CP026107.1 |
| Cupriavidus metallidurans | Genomic DNA | CP090526.1 |
| Cupriavidus metallidurans | Genomic DNA | CP083718.1 |
| Candidatus Accumulibacter similis | Genomic DNA | CP054595.1 |
| Aromatoleum petrolei | Genomic DNA | CP059560.1 |
| Rhodanobacteraceae bacterium | Genomic DNA | CP126120.1 |
| Paraburkholderia caribensis MBA4 | Genomic DNA | CP012747.1 |
| Sideroxydans sp. CL21 | Genomic DNA | LR699166.1 |
| Azoarcus sp. DN11 | Genomic DNA | CP021731.1 |
| Paraburkholderia terrae | Genomic DNA | AP025260.1 |
| Burkholderia thailandensis | Genomic DNA | CP023498.1 |
| Candidatus Symbiobacter mobilis CR | Genomic DNA | CP004885.1 |
| Burkholderia thailandensis MSMB121 | Genomic DNA | CP004096.1 |
| Paraburkholderia caribensis | Genomic DNA | CP065405.1 |
| Paraburkholderia phymatum STM815 | Genomic DNA | CP001045.1 |
| Methylococcus geothermalis | Genomic DNA | CP046565.1 |
| Variovorax sp. HW608 | Genomic DNA | LT607803.1 |
| Burkholderiales bacterium | Genomic DNA | CP070653.1 |
| Sulfuricella denitrificans skB26 | Genomic DNA | AP013066.1 |
| Burkholderia humptydooensis | Genomic DNA | CP013382.1 |
| Burkholderia sp. 2002721687 | Genomic DNA | CP009548.1 |
| Burkholderia humptydooensis | Genomic DNA | CP065687.1 |
| Thiomonas arsenitoxydans | Genomic DNA | FP475956.1 |

In one aspect, the DNA constructs disclosed herein include a gene that expresses a P-type ATPase. P-type ATPases are typically found in bacteria, archaea, and eukaryotes, and function as ion pumps and/or lipid pumps. P-type ATPases are also known as E1-E2 ATPases due to their interconversion between two forms (i.e., E1 and E2). P-type ATPases have a primarily α-helical structure and harness energy from ATP hydrolysis to transport a ligand across a cell membrane. Numerous P-type ATPases are recognized, typically classified into families based on affinity for particular ions (i.e., potassium, heavy metals, calcium, sodium/potassium, proton/potassium, sodium, proton, magnesium, and/or phospholipids).

In one aspect, the gene that encodes P-type ATPase is isolated from a bacterium such as, for example, Escherichia coli. In a further aspect, the gene that encodes P-type ATPase has SEQ ID NO. 12 or at least 70% homology thereto, at least 75% homology thereto, at least 80% homology thereto, at least 85% homology thereto, at least 90% homology thereto, at least 95% homology thereto, or at least 99% homology thereto.

Other sequences encoding P-type ATPase or related or homologous genes can be identified in a database such as, for example, GenBank. In one aspect, the gene that encodes chromate uranium reductase is isolated from Escherichia coli and can be identified by the GI number CP132223.1 in the GenBank database. In another aspect, sequences useful herein include those with GI numbers listed in Table 9:

TABLE 9
P-Type ATPase

| | Sequence | |
|---|---|---|
| Source Organism | Description | GI Number |
| Escherichia coli | Genomic DNA | CP132223.1 |
| Escherichia coli | Genomic DNA | CP137718.1 |
| Escherichia coli | Genomic DNA | CP137709.1 |
| Escherichia coli | Genomic DNA | OY754443.1 |
| Escherichia coli | Genomic DNA | OY754353.1 |
| Escherichia coli | Genomic DNA | OY754453.1 |
| Escherichia coli | Genomic DNA | CP107281.1 |
| Escherichia coli str. | Genomic DNA | CP044355.1 |
| K-12 substr. MG1655 | | |
| Escherichia coli | Genomic DNA | CP136397.1 |
| Escherichia coli | Genomic DNA | CP136396.1 |
| Escherichia coli | Genomic DNA | CP136398.1 |
| Escherichia coli | Genomic DNA | CP135502.1 |
| Escherichia coli | Genomic DNA | CP135077.1 |
| Escherichia coli | Genomic DNA | CP134923.1 |
| Escherichia coli | Genomic DNA | CP134918.1 |
| Escherichia coli | Genomic DNA | CP134910.1 |
| Escherichia coli | Genomic DNA | CP134370.1 |
| Escherichia coli | Genomic DNA | CP125679.1 |
| Escherichia coli | Genomic DNA | CP125676.1 |
| Escherichia coli | Genomic DNA | CP125687.1 |
| Escherichia coli | Genomic DNA | CP125681.1 |
| Escherichia coli | Genomic DNA | CP125699.1 |
| Escherichia coli | Genomic DNA | CP125688.1 |
| Escherichia coli | Genomic DNA | CP125691.1 |
| Escherichia coli | Genomic DNA | CP125695.1 |
| Escherichia coli | Genomic DNA | CP125701.1 |
| Escherichia coli | Genomic DNA | CP125702.1 |
| Escherichia coli | Genomic DNA | CP125710.1 |
| Escherichia coli | Genomic DNA | CP125713.1 |
| Escherichia coli | Genomic DNA | CP125715.1 |
| Escherichia coli | Genomic DNA | CP133854.1 |
| Escherichia coli | Genomic DNA | CP133453.1 |
| Escherichia coli | Genomic DNA | CP115361.1 |
| Escherichia coli | Genomic DNA | CP115333.1 |
| Escherichia coli | Genomic DNA | CP132594.1 |
| Escherichia coli | Genomic DNA | CP132550.1 |
| Escherichia coli | Genomic DNA | CP077969.2 |
| Escherichia coli | Genomic DNA | CP132288.1 |
| Escherichia coli | Genomic DNA | CP130710.1 |
| Escherichia coli | Genomic DNA | CP130706.1 |
| Escherichia coli | Genomic DNA | CP130447.1 |
| Escherichia coli | Genomic DNA | CP082100.1 |
| Escherichia coli | Genomic DNA | CP128790.1 |
| Escherichia coli | Genomic DNA | CP128609.1 |
| Escherichia coli | Genomic DNA | CP107005.1 |
| Escherichia coli | Genomic DNA | CP127119.1 |
| Escherichia coli | Genomic DNA | CP122496.1 |
| Escherichia coli | Genomic DNA | CP123255.1 |
| Escherichia coli | Genomic DNA | CP122317.1 |
| Escherichia coli | Genomic DNA | CP122315.1 |
| Escherichia coli | Genomic DNA | CP122316.1 |
| Escherichia coli | Genomic DNA | CP122318.1 |
| Escherichia coli | Genomic DNA | CP122319.1 |
| Escherichia coli | Genomic DNA | CP125731.1 |
| Escherichia coli | Genomic DNA | CP125621.1 |
| Escherichia coli | Genomic DNA | CP110018.1 |
| Escherichia coli | Genomic DNA | CP110017.1 |
| Escherichia coli | Genomic DNA | CP110016.1 |
| Escherichia coli | Genomic DNA | CP110015.1 |
| Escherichia coli | Genomic DNA | CP110014.1 |
| Escherichia coli | Genomic DNA | CP125059.1 |
| Escherichia coli | Genomic DNA | CP125009.1 |
| Escherichia coli | Genomic DNA | CP125039.1 |
| Escherichia coli | Genomic DNA | CP125045.1 |
| Escherichia coli | Genomic DNA | CP124995.1 |
| Escherichia coli | Genomic DNA | CP125003.1 |
| Escherichia coli | Genomic DNA | CP124986.1 |
| Escherichia coli | Genomic DNA | CP124994.1 |
| Escherichia coli | Genomic DNA | CP124970.1 |
| Escherichia coli | Genomic DNA | CP124979.1 |
| Escherichia coli | Genomic DNA | CP122625.1 |
| Escherichia coli | Genomic DNA | CP122832.1 |
| Escherichia coli | Genomic DNA | CP122872.1 |
| Escherichia coli | Genomic DNA | CP122876.1 |
| Escherichia coli | Genomic DNA | CP122895.1 |
| Escherichia coli | Genomic DNA | CP122923.1 |
| Escherichia coli | Genomic DNA | CP122929.1 |
| Escherichia coli | Genomic DNA | CP110117.1 |
| Escherichia coli | Genomic DNA | CP123237.1 |
| Escherichia coli | Genomic DNA | AP027953.1 |
| Escherichia coli | Genomic DNA | CP099032.1 |
| Escherichia coli | Genomic DNA | CP099034.1 |
| Escherichia coli | Genomic DNA | CP099062.1 |
| Escherichia coli | Genomic DNA | CP099065.1 |
| Escherichia coli | Genomic DNA | CP099087.1 |
| Escherichia coli | Genomic DNA | CP099092.1 |
| Escherichia coli | Genomic DNA | CP099095.1 |
| Escherichia coli | Genomic DNA | CP099106.1 |
| Escherichia coli | Genomic DNA | CP099118.1 |
| Escherichia coli | Genomic DNA | CP099179.1 |
| Escherichia coli | Genomic DNA | CP099294.1 |
| Escherichia coli | Genomic DNA | CP099027.1 |
| Escherichia coli | Genomic DNA | CP099059.1 |
| Escherichia coli | Genomic DNA | CP099068.1 |
| Escherichia coli | Genomic DNA | CP099072.1 |
| Escherichia coli | Genomic DNA | CP099075.1 |
| Escherichia coli | Genomic DNA | CP099089.1 |
| Escherichia coli | Genomic DNA | CP099166.1 |
| Escherichia coli | Genomic DNA | CP099178.1 |
| Escherichia coli | Genomic DNA | CP049112.1 |

In one aspect, the DNA constructs disclosed herein incorporate a gene that expresses tonB. TonB is a beta barrel protein found in the outer membrane of gram-negative bacteria. In a still further aspect, tonB proteins are involved with the uptake and/or transport of large substrates including iron siderophore complexes, heme, and other chelated forms of iron.

In one aspect, the gene that encodes tonB is isolated from a bacterium such as, for example, a Pseudomonas sp. In a further aspect, the gene that encodes tonB has SEQ ID NO. 13 or at least 70% homology thereto, at least 75% homology thereto, at least 80% homology thereto, at least 85% homology thereto, at least 90% homology thereto, at least 95% homology thereto, or at least 99% homology thereto.

Other sequences encoding tonB or related or homologous genes can be identified in a database such as, for example, GenBank. In one aspect, the gene that encodes chromate uranium reductase is isolated from Pseudomonas entomophila and can be identified by the GI number CP132921.1 in the GenBank database. In another aspect, sequences useful herein include those with GI numbers listed in Table 10:

TABLE 10
TonB

| | Sequence | |
|---|---|---|
| Source Organism | Description | GI Number |
| Pseudomonas entomophila | Genomic DNA | CP132921.1 |
| Pseudomonas entomophila L48 | Genomic DNA | CT573326.1 |
| Pseudomonas entomophila | Genomic DNA | CP070982.1 |
| Pseudomonas entomophila | Genomic DNA | CP063832.1 |
| Pseudomonas xantholysinigenes | Genomic DNA | CP077095.1 |
| Pseudomonas maumuensis | Genomic DNA | CP077077.1 |
| Pseudomonas fakonensis | Genomic DNA | CP077076.1 |
| Pseudomonas sp. CCOS 191 | Genomic DNA | LN847264.1 |
| Pseudomonas sichuanensis | Genomic DNA | CP087165.1 |
| Pseudomonas oryziphila | Genomic DNA | CP034337.1 |
| Pseudomonas oryziphila | Genomic DNA | CP034338.1 |
| Pseudomonas sp. 2hn | Genomic DNA | CP081016.1 |
| Pseudomonas xanthosomatis | Genomic DNA | CP077075.1 |
| Pseudomonas mosselii | Genomic DNA | CP103054.1 |
| Pseudomonas sichuanensis | Genomic DNA | CP087185.1 |
| Pseudomonas plecoglossicida | Genomic DNA | CP031146.1 |
| Pseudomonas mosselii | Genomic DNA | CP024159.1 |
| Pseudomonas mosselii | Genomic DNA | CP023299.1 |
| Pseudomonas mosselii | Genomic DNA | CP133092.1 |
| Pseudomonas mosselii | Genomic DNA | CP128544.1 |
| Pseudomonas mosselii | Genomic DNA | CP104107.1 |
| Pseudomonas mosselii | Genomic DNA | CP095556.1 |
| Pseudomonas plecoglossicida | Genomic DNA | CP050291.1 |
| Pseudomonas putida | Genomic DNA | CP014343.1 |
| Pseudomonas sp. RtlB026 | Genomic DNA | AP023348.2 |
| Pseudomonas mosselii | Genomic DNA | CP081966.1 |
| Pseudomonas muyukensis | Genomic DNA | CP077073.1 |
| Pseudomonas soli | Genomic DNA | CP083803.1 |
| Pseudomonas sp. PONIH3 | Genomic DNA | CP026386.1 |
| Pseudomonas soli | Genomic DNA | CP128543.1 |
| Pseudomonas sp. B21-023 | Genomic DNA | CP087190.1 |
| Pseudomonas sp. B21-044 | Genomic DNA | CP087172.1 |
| Pseudomonas soli | Genomic DNA | CP009365.1 |
| Pseudomonas sp. RC3H12 | Genomic DNA | CP075595.1 |
| Pseudomonas putida | Genomic DNA | CP018846.1 |
| Pseudomonas putida S13.1.2 | Genomic DNA | CP010979.1 |
| Pseudomonas fluorescens | Genomic DNA | CP100660.1 |
| Pseudomonas putida | Genomic DNA | AP022324.1 |
| Pseudomonas monteilii | Genomic DNA | CP040324.1 |
| Pseudomonas sp. LTGT-11-2Z | Genomic DNA | CP033104.1 |
| Pseudomonas monteilii | Genomic DNA | CP022562.1 |
| Pseudomonas putida | Genomic DNA | CP018743.1 |
| Pseudomonas monteilii | Genomic DNA | CP128547.1 |
| Pseudomonas monteilii | Genomic DNA | CP128546.1 |
| Pseudomonas monteilii | Genomic DNA | CP128541.1 |
| Pseudomonas monteilii | Genomic DNA | CP128545.1 |
| Pseudomonas monteilii | Genomic DNA | CP128549.1 |
| Pseudomonas shirazica | Genomic DNA | CP075569.1 |
| Pseudomonas monteilii | Genomic DNA | CP060595.1 |
| Pseudomonas putida | Genomic DNA | CP124529.1 |
| Pseudomonas asiatica | Genomic DNA | CP127872.1 |
| Pseudomonas putida | Genomic DNA | CP026115.2 |
| Pseudomonas sp. KU43P | Genomic DNA | AP019365.1 |
| Pseudomonas sp. 13159349 | Genomic DNA | CP045553.1 |
| Pseudomonas inefficax | Genomic DNA | CP134401.1 |
| Pseudomonas shirazica | Genomic DNA | CP127845.1 |
| Pseudomonas asiatica | Genomic DNA | CP128542.1 |
| Pseudomonas asiatica | Genomic DNA | CP107576.1 |
| Pseudomonas asiatica | Genomic DNA | CP101700.1 |
| Pseudomonas putida HB3267 | Genomic DNA | CP003738.1 |
| Pseudomonas shirazica | Genomic DNA | CP063456.1 |
| Pseudomonas asiatica | Genomic DNA | CP061848.1 |
| Pseudomonas asiatica | Genomic DNA | CP061335.1 |
| Pseudomonas putida | Genomic DNA | CP050951.1 |
| Pseudomonas sp. | Genomic DNA | CP119325.1 |
| Pseudomonas putida | Genomic DNA | CP096581.1 |
| Pseudomonas monteilii | Genomic DNA | CP014062.1 |
| Pseudomonas putida | Genomic DNA | LR813085.1 |
| Pseudomonas putida | Genomic DNA | LR813083.1 |
| Pseudomonas putida | Genomic DNA | CP137621.1 |
| Pseudomonas hunanensis | Genomic DNA | OY781045.1 |
| Pseudomonas putida | Genomic DNA | CP101910.1 |
| Pseudomonas putida | Genomic DNA | CP069080.1 |
| Pseudomonas sp. CIP-10 | Genomic DNA | CP087160.1 |
| Pseudomonas putida NBRC 14164 | Genomic DNA | AP013070.1 |
| Pseudomonas putida | Genomic DNA | CP046872.1 |
| Pseudomonas monteilii | Genomic DNA | CP043396.1 |
| Pseudomonas monteilii | Genomic DNA | CP043395.1 |
| Pseudomonas sp. NBB | Genomic DNA | CP103994.1 |
| Pseudomonas kurunegalensis | Genomic DNA | CP128548.1 |
| Pseudomonas asiatica | Genomic DNA | CP128558.1 |
| Pseudomonas asiatica | Genomic DNA | CP084714.1 |
| Pseudomonas putida | Genomic DNA | CP109606.1 |
| Pseudomonas sp. A2 | Genomic DNA | CP039127.1 |
| Pseudomonas sp. FGI182 | Genomic DNA | CP007012.1 |
| Pseudomonas putida S16 | Genomic DNA | CP002870.1 |
| Pseudomonas putida GB-1 | Genomic DNA | CP000926.1 |
| Pseudomonas putida | Genomic DNA | CP045551.1 |
| Pseudomonas sp. XWY-1 | Genomic DNA | CP026332.1 |
| Pseudomonas sp. BO3-4 | Genomic DNA | CP139018.1 |
| Pseudomonas hunanensis | Genomic DNA | CP131127.1 |
| Pseudomonas sp. JY-Q | Genomic DNA | CP011525.1 |
| Pseudomonas putida | Genomic DNA | CP120969.1 |
| Pseudomonas putida | Genomic DNA | CP096920.1 |
| Pseudomonas putida DOT-T1E | Genomic DNA | CP110782.1 |
| Pseudomonas sp. HD6515 | Genomic DNA | CP079827.1 |
| Pseudomonas putida | Genomic DNA | CP047311.1 |
| Pseudomonas putida DOT-T1E | Genomic DNA | CP003734.1 |
| Pseudomonas sp. CFA | Genomic DNA | CP044546.1 |
| Pseudomonas putida | Genomic DNA | CP043576.1 |

In one aspect, the DNA constructs disclosed herein incorporate a gene that encodes a heat shock protein. In a further aspect, heat shock proteins are a group of proteins produced by cells in response to exposure to stressful conditions. In a further aspect, heat shock proteins can be expressed in response to heat shock but also to cold, UV light, wound healing, exposure to toxic chemicals such as, for example heavy metals, as well as during tissue remodeling. In a further aspect, a heat shock protein may function as a chaperone protein by assisting in the refolding process of proteins damaged by cell stress. In a further aspect, the heat shock protein can be HSP60, HSP70, or HSP90, where the number refers to the size in kilodaltons of the protein. In one aspect, the heat shock protein is HSP70 and has a weight of about 70 kDa.

In one aspect, the gene that encodes a heat shock protein is isolated from a yeast such as, for example, Saccharomyces cerevisiae. In a further aspect, the gene that encodes a heat shock protein has SEQ ID NO. 14 or at least 70% homology thereto, at least 75% homology thereto, at least 80% homology thereto, at least 85% homology thereto, at least 90% homology thereto, at least 95% homology thereto, or at least 99% homology thereto.

Other sequences encoding HSP70 or related or homologous genes can be identified in a database such as, for example, GenBank. In one aspect, the gene that encodes chromate uranium reductase is isolated from Saccharomyces cerevisiae and can be identified by the GI number CP046084.1 in the GenBank database. In another aspect, sequences useful herein include those with GI numbers listed in Table 11:

TABLE 11
Heat Shock Protein 70

| Source Organism | Sequence Description | GI Number |
|---|---|---|
| Saccharomyces cerevisiae | Genomic DNA | CP046084.1 |
| Saccharomyces cerevisiae | Genomic DNA | CP036483.1 |
| Saccharomyces cerevisiae | Genomic DNA | CP033473.1 |
| Saccharomyces cerevisiae | Genomic DNA | CP029160.1 |
| Saccharomyces cerevisiae | Genomic DNA | CP026298.1 |
| Saccharomyces cerevisiae | Genomic DNA | CP022969.1 |
| Saccharomyces cerevisiae | Genomic DNA | CP020126.1 |
| Saccharomyces cerevisiae YJM1326 | Genomic DNA | CP004710.2 |
| Saccharomyces cerevisiae | Genomic DNA | CP127198.1 |
| Saccharomyces cerevisiae | Genomic DNA | CP011550.1 |
| Saccharomyces cerevisiae | Genomic DNA | AP026835.1 |
| Saccharomyces cerevisiae | Genomic DNA | CP011671.1 |
| Saccharomyces cerevisiae | Genomic DNA | CP089103.1 |
| Saccharomyces paradoxus | Genomic DNA | CP081969.2 |
| Saccharomyces cerevisiae | Genomic DNA | CP063258.1 |
| Saccharomyces cerevisiae | Genomic DNA | CP059525.2 |
| Saccharomyces cerevisiae S288C | Genomic DNA | BK006938.2 |
| Saccharomyces cerevisiae S288C | Genomic DNA | NM_001180289.1 |
| Saccharomyces cerevisiae EC1118 | Genomic DNA | FN393064.1 |
| Saccharomyces cerevisiae | Genomic DNA | Z74277.1 |
| Saccharomyces cerevisiae | Heat shock cognate gene | X13713.1 |
| synthetic construct | Genomic DNA | EF058944.1 |
| Saccharomyces cerevisiae | Genomic DNA | CP025100.1 |
| Saccharomyces cerevisiae | Genomic DNA | CP020228.1 |
| Saccharomyces cerevisiae | Genomic DNA | CP020160.1 |
| Saccharomyces cerevisiae YJM1447 | Genomic DNA | CP004738.2 |
| Saccharomyces cerevisiae YJM981 | Genomic DNA | CP004688.2 |
| Saccharomyces cerevisiae YJM627 | Genomic DNA | CP004678.2 |
| Saccharomyces cerevisiae YJM1401 | Genomic DNA | CP004727.2 |
| Saccharomyces cerevisiae YJM1356 | Genomic DNA | CP004717.2 |
| Saccharomyces cerevisiae YJM978 | Genomic DNA | CP004687.2 |
| Saccharomyces cerevisiae YJM320 | Genomic DNA | CP004667.2 |
| Saccharomyces cerevisiae YJM1527 | Genomic DNA | CP004746.2 |
| Saccharomyces cerevisiae YJM1355 | Genomic DNA | CP004716.2 |
| Saccharomyces cerevisiae YJM554 | Genomic DNA | CP004676.2 |
| Saccharomyces cerevisiae | Genomic DNA | CP008239.1 |
| Saccharomyces cerevisiae | Genomic DNA | CP008324.1 |
| Saccharomyces cerevisiae | Genomic DNA | CP008273.1 |
| Saccharomyces cerevisiae | Genomic DNA | CP008256.1 |
| Saccharomyces cerevisiae | Genomic DNA | CP008222.1 |
| Saccharomyces cerevisiae | Genomic DNA | CP008409.1 |
| Saccharomyces cerevisiae | Genomic DNA | CP008392.1 |
| Saccharomyces cerevisiae | Genomic DNA | CP008375.1 |
| Saccharomyces cerevisiae | Genomic DNA | CP008358.1 |
| Saccharomyces cerevisiae | Genomic DNA | CP008341.1 |
| Saccharomyces cerevisiae | Genomic DNA | CP008494.1 |
| Saccharomyces cerevisiae | Genomic DNA | CP008443.1 |
| Saccharomyces cerevisiae | Genomic DNA | CP008579.1 |
| Saccharomyces cerevisiae | Genomic DNA | CP008511.1 |
| Saccharomyces cerevisiae | Genomic DNA | CP008647.1 |
| Saccharomyces cerevisiae | Genomic DNA | CP008630.1 |
| Saccharomyces cerevisiae | Genomic DNA | CP008596.1 |
| Saccharomyces cerevisiae | Genomic DNA | CP008188.1 |
| Saccharomyces cerevisiae | Genomic DNA | CP008171.1 |
| Saccharomyces cerevisiae | Genomic DNA | CP008154.1 |
| Saccharomyces cerevisiae | Genomic DNA | CP008681.1 |
| Saccharomyces cerevisiae | Genomic DNA | CP008137.1 |
| Saccharomyces cerevisiae | Genomic DNA | CP008120.1 |
| Saccharomyces cerevisiae | Genomic DNA | CP008086.1 |
| Saccharomyces cerevisiae | Genomic DNA | CP008052.1 |
| Saccharomyces cerevisiae | Genomic DNA | CP008035.1 |
| Saccharomyces cerevisiae | Genomic DNA | CP008001.1 |
| Saccharomyces cerevisiae | Genomic DNA | CP007984.1 |
| Saccharomyces cerevisiae | Genomic DNA | CP007950.1 |
| Saccharomyces cerevisiae | Genomic DNA | CP007899.1 |
| Saccharomyces cerevisiae | Genomic DNA | CP007882.1 |
| Saccharomyces cerevisiae | Genomic DNA | CP007831.1 |
| Saccharomyces cerevisiae | Genomic DNA | CP133024.1 |
| Saccharomyces cerevisiae YJM1526 | Genomic DNA | CP004745.2 |
| Saccharomyces cerevisiae YJM969 | Genomic DNA | CP004684.2 |
| Saccharomyces cerevisiae YJM1478 | Genomic DNA | CP004743.2 |
| Saccharomyces cerevisiae YJM1338 | Genomic DNA | CP004713.2 |
| Saccharomyces cerevisiae YJM993 | Genomic DNA | CP004692.2 |
| Saccharomyces cerevisiae YJM1477 | Genomic DNA | CP004742.2 |
| Saccharomyces cerevisiae YJM1387 | Genomic DNA | CP004722.2 |
| Saccharomyces cerevisiae YJM453 | Genomic DNA | CP004672.2 |
| Saccharomyces cerevisiae YJM1242 | Genomic DNA | CP004701.2 |
| Saccharomyces cerevisiae YJM683 | Genomic DNA | CP004681.2 |
| Saccharomyces cerevisiae YJM987 | Genomic DNA | CP004690.2 |
| Saccharomyces cerevisiae YJM450 | Genomic DNA | CP004670.2 |
| Saccharomyces cerevisiae | Genomic DNA | CP011082.1 |
| Saccharomyces cerevisiae YJM1415 | Genomic DNA | CP004729.1 |
| Saccharomyces cerevisiae | Genomic DNA | CP072078.1 |
| Saccharomyces cerevisiae | Genomic DNA | CP072094.1 |
| Saccharomyces cerevisiae | Genomic DNA | CP093816.1 |
| Saccharomyces cerevisiae | Genomic DNA | CP093752.1 |
| Saccharomyces cerevisiae | Genomic DNA | CP093704.1 |
| Saccharomyces cerevisiae | Genomic DNA | CP093688.1 |
| Saccharomyces cerevisiae | Genomic DNA | CP093672.1 |
| Saccharomyces cerevisiae | Genomic DNA | CP093656.1 |
| Saccharomyces cerevisiae | Genomic DNA | CP093624.1 |
| Saccharomyces cerevisiae | Genomic DNA | CP093576.1 |
| Saccharomyces cerevisiae | Genomic DNA | CP093560.1 |
| Saccharomyces cerevisiae | Genomic DNA | LR813588.2 |
| Saccharomyces cerevisiae | Genomic DNA | LR813537.2 |
| Saccharomyces cerevisiae | Heat shock protein 70 | M25395.1 |
| Saccharomyces cerevisiae | Genomic DNA | CP046471.1 |
| Saccharomyces cerevisiae | Genomic DNA | CP125417.1 |
| Saccharomyces cerevisiae | Genomic DNA | CP125400.1 |
| Saccharomyces cerevisiae | Genomic DNA | CP080606.1 |

In one aspect, the DNA constructs disclosed herein include a gene that expresses ribulose-1,5-bisphosphate carboxylase/oxygenase (RuBisCO) large subunit 1. RuBisCO is an enzyme involved in carbon fixation and can be isolated from plants, algae, cyanobacteria, and phototrophic and/or chemoautotrophic proteobacteria. In a further aspect, the RuBisCO large subunit is typically encoded by chloroplast DNA. In a still further aspect, RuBisCO catalyzes the formation of two molecules of glycerate-3-phosphate from ribulose-1,5-bisphosphate and carbon dioxide. In an alternative aspect, RuBisCO is capable of catalyzing the formation of phosphoglycolate and 3-phosphoglycerate from ribulose-1,5-bisphosphate and molecular oxygen.

In one aspect, the gene that encodes RuBisCO large subunit 1 is isolated from an algae, protist, or stramenophile. In a further aspect, the gene that encodes RuBisCO large subunit 1 has SEQ ID NO. 15 or at least 70% homology thereto, at least 75% homology thereto, at least 80% homology thereto, at least 85% homology thereto, at least 90% homology thereto, at least 95% homology thereto, or at least 99% homology thereto.

Other sequences encoding RuBisCO large subunit 1 or related or homologous genes can be identified in a database such as, for example, GenBank. In one aspect, the gene that encodes chromate uranium reductase is isolated from Guillardia theta and can be identified by the GI number NC_000926.1 in the GenBank database. In another aspect, sequences useful herein include those with GI numbers listed in Table 12:

TABLE 12
RuBisCO Large Subunit

| Source Organism | Sequence Description | GI Number |
|---|---|---|
| Guillardia theta | Chloroplast genomic DNA | NC_000926.1 |
| Hanusia phi | RuBisCO large subunit | MG646607.1 |
| Guillardia theta | RuBisCO large subunit | MK818447.1 |
| Guillardia theta | Chloroplast genomic DNA | KT428890.1 |
| Storeatula sp. CCMP1868 | Chloroplast genomic DNA | KY856940.1 |
| Cryptomonas sp. NIES-3952 | Chloroplast genomic DNA | LC648965.1 |
| Rhodomonas salina | Chloroplast genomic DNA | NC_009573.1 |
| Cryptomonas pyrenoidifera | Chloroplast genomic DNA | NC_069042.1 |
| Rhodomonas sp. NIES-1730 | Chloroplast genomic DNA | LC648961.1 |
| Cryptomonas curvata | Chloroplast genomic DNA | LC648951.1 |
| Rhodomonas sp. NIES-698 | Chloroplast genomic DNA | LC648954.1 |
| Cryptomonas tetrapyrenoidosa | Chloroplast genomic DNA | LC648953.1 |
| Rhodomonas sp. NIES-2332 | Chloroplast genomic DNA | LC648963.1 |
| Cryptomonas curvata | Chloroplast genomic DNA | LC484192.1 |
| Teleaulax amphioxeia | Chloroplast genomic DNA | NC_027589.1 |
| Hanusia phi | RuBisCO large subunit | KX777654.1 |
| Cryptomonas curvata | Chloroplast genomic DNA | NC_035720.1 |
| Chroomonas debatzensis | Chloroplast genomic DNA | LC648962.1 |
| Chroomonas collegionis | Chloroplast genomic DNA | LC648955.1 |
| Rhodomonas sp. NIES-1006 | Chloroplast genomic DNA | LC648958.1 |
| Cryptophyta sp. CCMP2293 | Chloroplast genomic DNA | MK798155.1 |
| Hemiselmis andersenii | Chloroplast genomic DNA | LC648964.1 |
| Chroomonas placoidea | Chloroplast genomic DNA | NC_035721.1 |
| Chroomonas mesostigmatica | Chloroplast genomic DNA | KY860574.1 |
| CCMP1168 | | |
| Cryptomonas gyropyrenoidosa | Chloroplast genomic DNA | NC_077586.1 |
| Proteomonas sp. NIES-1005 | Chloroplast genomic DNA | LC648957.1 |
| Proteomonas sp. NEIS-1375 | Chloroplast genomic DNA | LC648960.1 |
| Cryptomonas sp. | RuBisCO large subunit | MG646601.1 |
| Rhodomonas sp. CCMP760 | RuBisCO large subunit | MG646616.1 |
| Rhodomonas salina | RuBisCO large subunit | MG646611.1 |
| Rhodomonas sp. | RuBisCO large subunit | MG646613.1 |
| Cryptomonas pyrenoidifera | RuBisCO large subunit | AM051217.1 |
| Rhodomonas sp. | RuBisCO large subunit | MG646614.1 |
| Rhodomonas sp. | RuBisCO large subunit | MG646612.1 |
| Cryptomonas borealis | Chloroplast genomic DNA | LC648950.1 |
| Cryptomonas marssonii | RuBisCO large subunit | AM051209.1 |
| Cryptomonas obovoidea | RuBisCO large subunit | AM051223.1 |
| Cryptomonas tetrapyrenoidosa | RuBisCO large subunit | AM051219.1 |
| Cryptomonas erosa | RuBisCO large subunit | MG646599.1 |
| Cryptomonas erosa | RuBisCO large subunit | MG646600.1 |
| Cryptomonas pyrenoidifera | RuBisCO large subunit | AM051216.1 |
| Cryptophyta sp. ECY-2019a | RuBisCO large subunit | MG646606.1 |
| Cryptomonas tetrapyrenoidosa | RuBisCO large subunit | AM051220.1 |
| Cryptomonas curvata | RuBisCO large subunit | AM051204.1 |
| Cryptomonas curvata | RuBisCO large subunit | AM051205.1 |
| Cryptomonas obovoidea | RuBisCO large subunit | AM051221.1 |
| Cryptomonas sp. CCAC 0109 | RuBisCO large subunit | AM051222.1 |
| Cryptomonas erosa | RuBisCO large subunit | MG646598.1 |
| Cryptomonas sp. NIES-1327 | Chloroplast genomic DNA | LC648959.1 |
| Cryptomonas sp. NIES-345 | Chloroplast genomic DNA | LC648952.1 |
| Rhodomonas sp. CCMP740 | RuBisCO large subunit | MG646615.1 |
| Hemiselmis tepida | RuBisCO large subunit | MG646605.1 |
| Pseudoerythrocladia kornmannii | Chloroplast genomic DNA | NC_062386.1 |
| Pseudoerythrocladia kornmannii | Chloroplast genomic DNA | MW675664.1 |
| Rhodomonas sp. | RuBisCO large subunit | MG646617.1 |
| Hemiselmis andersenii | RuBisCO large subunit | MG646603.1 |
| Cryptomonas ovata | RuBisCO large subunit | AM051210.1 |
| Botrydiopsis sp. | Photosystem II protein D1 | MK909755.1 |
| Erythrolobus coxiae | Chloroplast genomic DNA | NC_062391.1 |
| Heterothrix mucicola | Photosystem II protein D1 | EF455957.1 |
| Erythrotrichia foliiformis | Chloroplast genomic DNA | MW675678.1 |
| Cryptophyta sp. CCMP2293 | RuBisCO large subunit | MG646618.1 |
| Ophiocytium majus | RuBisCO large subunit | MK482708.1 |
| Ophiocytium parvulum | RuBisCO large subunit | MK482697.1 |
| Hemiselmis cryptochromatica | RuBisCO large subunit | MG646604.1 |
| Heterothrix mucicola | Photosystem II protein D1 | MK804169.1 |
| Xanthonema sp. SAG 60.94 | Photosystem II protein D1 | EF455977.1 |
| Botrydium granulatum | Photosystem II protein D1 | EF455980.1 |
| Botrydium stoloniferum | RuBisCO large subunit | AF465707.1 |
| Xanthonema sp. SAG 2189 | Photosystem II protein D1 | EF455954.1 |
| Porphyridium aerugineum | Chloroplast genomic DNA | NC_062300.1 |
| Xanthonema sp. SAG 2192 | Genomic DNA | EF426794.1 |
| Xanthonema cf. hormidioides | Photosystem II protein D1 | EF455953.1 |
| Porphyridium aerugineum | RuBisCO large subunit | X17597.1 |
| Cryptomonas marssonii | RuBisCO large subunit | AM051208.1 |
| Xanthonema bristolianum | Photosystem II protein D1 | MK792448.1 |
| Xanthonema bristolianum | Photosystem II protein D1 | MW176120.1 |
| Xanthonema bristolianum | Photosystem II protein D1 | MW176119.1 |
| Xanthonema bristolianum | Photosystem II protein D1 | MK804168.1 |
| Xanthonema bristolianum | Photosystem II protein D1 | MK804167.1 |
| Xanthonema bristolianum | Photosystem II protein D1 | MK804166.1 |
| Botrydium stoloniferum | Photosystem II protein D1 | EF455981.1 |
| Xanthonema sp. CCAP 836/5 | Photosystem II protein D1 | EF455940.1 |
| Xanthonema exile | Photosystem II protein D1 | EF455929.1 |
| Xanthonema bristolianum | Photosystem II protein D1 | EF455955.1 |
| Xanthonema exile | Photosystem II protein D1 | EF455937.1 |
| Ophiocytium majus | RuBisCO large subunit | MK482707.1 |
| Ophiocytium sp. | RuBisCO large subunit | MK482706.1 |
| Xanthonema solidum | Photosystem II protein D1 | EF455973.1 |
| Ophiocytium majus | Photosystem II protein D1 | EF455971.1 |
| Chrysoparadoxa australica | RuBisCO large subunit | MK189080.1 |
| Ophiocytium majus | RuBisCO large subunit | MK482705.1 |
| Sahlingia subintegra | Chloroplast genomic DNA | NC_062389.1 |
| Rhodaphanes brevistipitata | Chloroplast genomic DNA | NC_062393.1 |
| Xanthonema sp. SAG 2179 | Photosystem II protein D1 | EF426796.1 |
| Xanthonema debile | Photosystem II protein D1 | EF455975.1 |
| Xanthonema hormidioides | Photosystem II protein D1 | EF455939.1 |
| Xanthonema hormidioides | Photosystem II protein D1 | EF455922.1 |
| Bumilleriopsis sp. SAG 33.93 | Photosystem II protein D1 | EF431849.1 |
| Botrydium sp. | Photosystem II protein D1 | MW176112.1 |

In one aspect, the DNA constructs disclosed herein incorporate a gene that encodes phosphoenolpyruvate carboxylase. In a further aspect, phosphoenolpyruvate carboxylase is an enzyme that catalyzes the addition of bicarbonate to phosphoenolpyruvate to form oxaloacetate; inorganic phosphate is also a product of this reaction. Phosphoenolpyruvate carboxylase is used for carbon fixation in C₄ plants, crassulacean acid metabolism plants, and certain bacteria.

In one aspect, the gene that encodes phosphoenolpyruvate carboxylase is isolated from a plant. In a further aspect, the gene that encodes phosphoenolpyruvate carboxylase has SEQ ID NO. 16 or at least 70% homology thereto, at least 75% homology thereto, at least 80% homology thereto, at least 85% homology thereto, at least 90% homology thereto, at least 95% homology thereto, or at least 99% homology thereto.

Other sequences encoding phosphoenolpyruvate carboxylase or related or homologous genes can be identified in a database such as, for example, GenBank. In one aspect, the gene that encodes chromate uranium reductase is isolated from Brassica napus and can be identified by the GI number XM_048750595 in the GenBank database. In another aspect, sequences useful herein include those with GI numbers listed in Table 13:

TABLE 13
Phosphoenolpyruvate Carboxylase 1

| Source Organism | Sequence Description | GI Number |
|---|---|---|
| Brassica napus | Phosphoenolpyruvate carboxylase 1 | XM_048750595.1 |
| Brassica napus | Phosphoenolpyruvate carboxylase 1 | XM_048750594.1 |
| Brassica napus | Phosphoenolpyruvate carboxylase 1 | XM_048750593.1 |
| Brassica napus | Phosphoenolpyruvate carboxylase 1 | XM_048750592.1 |
| Brassica napus | Phosphoenolpyruvate carboxylase 1 | XM_048750591.1 |
| Brassica napus | Phosphoenolpyruvate carboxylase 1 | XM_048750590.1 |
| Brassica napus | Phosphoenolpyruvate carboxylase 1 | XM_013800011.3 |
| Brassica napus | Phosphoenolpyruvate carboxylase 1 | XM_048750588.1 |
| Arabidopsis thaliana | Phosphoenolpyruvate carboxylase 1 | NM_001036102.3 |
| Arabidopsis thaliana | Phosphoenolpyruvate carboxylase 1 | NM_001036101.2 |
| Arabidopsis thaliana | Phosphoenolpyruvate carboxylase 1 | NM_104209.3 |
| Arabidopsis thaliana | Phosphoenolpyruvate carboxylase 1 | AJ532901.1 |
| Arabidopsis thaliana | mRNA | AY057507.1 |
| Arabidopsis thaliana | mRNA | AK317277.1 |
| Brassica oleracea var. | Phosphoenolpyruvate carboxylase 1 | XM_013773407.1 |
| oleracea | | |
| Arabidopsis lyrata subsp. | Phosphoenolpyruvate carboxylase 1 | XM_021011473.1 |
| lyrata | | |
| Arabidopsis lyrata subsp. | Phosphoenolpyruvate carboxylase 1 | XM_021011472.1 |
| lyrata | | |
| Brassica rapa | Phosphoenolpyruvate carboxylase 1 | XM_009108735.3 |
| Brassica napus | Phosphoenolpyruvate carboxylase 1 | XM_013817493.3 |
| Brassica juncea | Phosphoenolpyruvate carboxylase 1 | AJ223497.1 |
| Camelina sativa | Phosphoenolpyruvate carboxylase 1 | XM_019239140.1 |
| Raphanus sativus | Phosphoenolpyruvate carboxylase 1 | XM_057000440.1 |
| Camelina sativa | Phosphoenolpyruvate carboxylase 1 | XM_010481584.1 |
| Brassica juncea | Phosphoenolpyruvate carboxylase 1 | AJ223496.1 |
| Capsella rubella | Phosphoenolpyruvate carboxylase 1 | XM_006306616.2 |
| Capsella rubella | Phosphoenolpyruvate carboxylase 1 | XM_006306617.2 |
| Camelina sativa | Phosphoenolpyruvate carboxylase 1 | XM_010463911.2 |
| Camelina sativa | Phosphoenolpyruvate carboxylase 1 | XM_010463909.2 |
| Camelina sativa | Phosphoenolpyruvate carboxylase 1 | XM_010463910.2 |
| Camelina sativa | Phosphoenolpyruvate carboxylase 1-like | XM_010502669.2 |
| Camelina sativa | Phosphoenolpyruvate carboxylase 1-like | XM_010502668.2 |
| Eutrema halophilum | mRNA | AK353394.1 |
| Brassica napus | Phosphoenolpyruvate carboxylase 1-like | XM_048760693.1 |
| Brassica napus | Phosphoenolpyruvate carboxylase 1-like | XM_048760692.1 |
| Eutrema salsugineum | Phosphoenolpyruvate carboxylase 1 | XM_006392739.2 |
| Eutrema salsugineum | Phosphoenolpyruvate carboxylase 1 | XM_024150185.1 |
| Brassica juncea | Phosphoenolpyruvate carboxylase 1 | KX352389.1 |
| Tarenaya hassleriana | Phosphoenolpyruvate carboxylase 3 | XM_010553816.2 |
| Tarenaya hassleriana | Phosphoenolpyruvate carboxylase 3 | XM_010553815.2 |
| Tarenaya hassleriana | Phosphoenolpyruvate carboxylase 1-like | XM_010544006.1 |
| Tarenaya hassleriana | Phosphoenolpyruvate carboxylase 1-like | XM_010529078.2 |
| Tarenaya hassleriana | Phosphoenolpyruvate carboxylase 1-like | XM_019201455.1 |
| Brassica napus | Phosphoenolpyruvate carboxylase 3 | XM_013880828.3 |
| Brassica napus | Phosphoenolpyruvate carboxylase 3 | XM_013880829.3 |
| Eutrema salsugineum | Phosphoenolpyruvate carboxylase 3 | XM_006406967.2 |
| Capsella rubella | Phosphoenolpyruvate carboxylase 3 | XM_006296845.2 |
| Capsella rubella | Phosphoenolpyruvate carboxylase 3 | XM_023786107.1 |
| Eutrema halophilum | mRNA | AK353350.1 |
| Camelina sativa | Phosphoenolpyruvate carboxylase 3 | XM_019246348.1 |
| Camelina sativa | Phosphoenolpyruvate carboxylase 3-like | XM_010467124.1 |
| Gossypium hirsutum | Phosphoenolpyruvate carboxylase | XM_041086879.1 |
| Gossypium raimondii | Phosphoenolpyruvate carboxylase | XM_012601897.2 |
| Arabidopsis lyrata subsp. | Phosphoenolpyruvate carboxylase 3 | XM_002882858.2 |
| lyrata | | |
| Gossypium arboreum | Phosphoenolpyruvate carboxylase | XM_017786535.2 |
| Gossypium hirsutum | Phosphoenolpyruvate carboxylase | XM_041075041.1 |
| Brassica rapa | Phosphoenolpyruvate carboxylase 3 | XM_009137165.3 |
| Hibiscus syriacus | Phosphoenolpyruvate carboxylase | XM_039166986.1 |
| Brassica rapa | Phosphoenolpyruvate carboxylase 3 | XM_033292762.1 |
| Brassica rapa | Phosphoenolpyruvate carboxylase 3 | XM_009148057.3 |
| Brassica rapa | Phosphoenolpyruvate carboxylase 3 | XM_009148056.2 |
| Brassica napus | Phosphoenolpyruvate carboxylase 3-like | XM_013838397.3 |
| Arabidopsis thaliana | Phosphoenolpyruvate carboxylase 3 | NM_112356.4 |
| Arabidopsis thaliana | Phosphoenolpyruvate carboxylase 3 | NM_001338141.1 |
| Arabidopsis thaliana | Phosphoenolpyruvate carboxylase | AF071788.1 |
| Hibiscus syriacus | Phosphoenolpyruvate carboxylase-like | XM_039145669.1 |
| Cucurbita moschata | Phosphoenolpyruvate carboxylase | XM_023084601.1 |
| Cucurbita moschata | Phosphoenolpyruvate carboxylase | XM_023084600.1 |
| Cucurbita moschata | Phosphoenolpyruvate carboxylase | XM_023084599.1 |
| Brassica oleracea var. | Phosphoenolpyruvate carboxylase 3 | XM_013782833.1 |
| oleracea | | |
| Theobroma cacao | Phosphoenolpyruvate carboxylase | XM_007021667.2 |
| Theobroma cacao | Phosphoenolpyruvate carboxylase | XM_007021666.2 |
| Arabidopsis thaliana | Phosphoenolpyruvate carboxylase | AK227556.1 |
| Arabidopsis thaliana | Genomic DNA | BT004642.1 |
| Gossypium hirsutum | Phosphoenolpyruvate carboxylase | EU032328.1 |
| Brassica napus | Phosphoenolpyruvate carboxylase 3-like | XM_048779837.1 |
| Brassica napus | Phosphoenolpyruvate carboxylase 3-like | XM_048779836.1 |
| Raphanus sativus | Phosphoenolpyruvate carboxylase 3 | XM_056985607.1 |
| Vitis riparia | Phosphoenolpyruvate carboxylase | XM_034822307.1 |
| Vitis riparia | Phosphoenolpyruvate carboxylase | XM_034822306.1 |
| Ziziphus jujuba | Phosphoenolpyruvate carboxylase | XM_016043691.4 |
| Cucurbita moschata | Phosphoenolpyruvate carboxylase | XM_023080896.1 |
| Rhodamnia argentea | Phosphoenolpyruvate carboxylase | XM_030694355.2 |
| Mangifera indica | Phosphoenolpyruvate carboxylase | XM_044617504.1 |
| Citrus sinensis | Phosphoenolpyruvate carboxylase | XM_006487390.4 |
| Citrus sinensis | Phosphoenolpyruvate carboxylase | XM_006487389.3 |
| Momordica charantia | Phosphoenolpyruvate carboxylase | XM_022303837.1 |
| Momordica charantia | Phosphoenolpyruvate carboxylase | XM_022303828.1 |
| Citrus x clementina | Phosphoenolpyruvate carboxylase | XM_024187754.1 |
| Citrus x clementina | Phosphoenolpyruvate carboxylase | XM_006442451.2 |
| Vitis vinifera | Phosphoenolpyruvate carboxylase | XM_002285405.4 |
| Herrania umbratica | Phosphoenolpyruvate carboxylase | XM_021431888.1 |
| Cucurbita pepo subsp. pepo | Phosphoenolpyruvate carboxylase | XM_023668092.1 |
| Cucurbita pepo subsp. pepo | Phosphoenolpyruvate carboxylase | XM_023668091.1 |
| Cucurbita pepo subsp. pepo | Phosphoenolpyruvate carboxylase | XM_023668090.1 |
| Cucurbita pepo subsp. pepo | Phosphoenolpyruvate carboxylase | XM_023679479.1 |
| Morus notabilis | Phosphoenolpyruvate carboxylase | XM_024173393.1 |
| Oryza brachyantha | Phosphoenolpyruvate carboxylase 2-like | XM_006659256.2 |
| Pyrus x bretschneideri | Phosphoenolpyruvate carboxylase | XM_009341762.3 |
| Syzygium oleosum | Phosphoenolpyruvate carboxylase | XM_030605022.2 |
| Eucalyptus grandis | Phosphoenolpyruvate carboxylase | XM_010062388.3 |

In one aspect, the DNA constructs disclosed herein incorporate a gene that encodes phosphoenol pyruvate carboxykinase. In a further aspect, phosphoenol pyruvate carboxykinase is an enzyme used in the gluconeogenesis metabolic pathway. Phosphoenol pyruvate carboxykinase in the cytosol converts oxaloacetate into phosphoenol pyruvate and carbon dioxide in the presence of GTP in an irreversible reaction. Mitochondrial isoforms also exist and can be transported to the cytosol. Plants and bacteria also encode phosphoenol pyruvate carboxykinase enzymes.

In one aspect, the gene that encodes phosphoenol pyruvate carboxykinase is isolated from a mammal. In a further aspect, the gene that encodes phosphoenol pyruvate carboxykinase has SEQ ID NO. 17 or at least 70% homology thereto, at least 75% homology thereto, at least 80% homology thereto, at least 85% homology thereto, at least 90% homology thereto, at least 95% homology thereto, or at least 99% homology thereto.

Other sequences encoding phosphoenol pyruvate carboxykinase or related or homologous genes can be identified in a database such as, for example, GenBank. In one aspect, the gene that encodes chromate uranium reductase is isolated from Homo sapiens and can be identified by the GI number NM_002591.4 in the GenBank database. In another aspect, sequences useful herein include those with GI numbers listed in Table 14:

TABLE 14
Phosphoenol Pyruvate Carboxykinase

| Source Organism | Sequence Description | GI Number |
|---|---|---|
| Homo sapiens | Phosphoenolpyruvate carboxykinase 1 | NM_002591.4 |
| Homo sapiens | Similar to phosphoenolpyruvate | AK290802.1 |
| | carboxykinase 1 | |
| synthetic construct | Phosphoenolpyruvate carboxykinase 1 | DQ896497.2 |
| synthetic construct | Phosphoenolpyruvate carboxykinase 1 | DQ893518.2 |
| Homo sapiens | Phosphoenolpyruvate carboxykinase 1 | BC023978.1 |
| synthetic construct | Phosphoenolpyruvate carboxykinase 1 | AY888078.1 |
| Pan troglodytes | Phosphoenolpyruvate carboxykinase 1 | XM_514745.8 |
| Homo sapiens | Phosphoenolpyruvate carboxykinase 1 | L05144.1 |
| Pan paniscus | Phosphoenolpyruvate carboxykinase 1 | XM_003806166.5 |
| Gorilla gorilla gorilla | Phosphoenolpyruvate carboxykinase 1 | XM_004062425.4 |
| Symphalangus syndactylus | Phosphoenolpyruvate carboxykinase 1 | XM_055265768.1 |
| Pongo pygmaeus | Phosphoenolpyruvate carboxykinase 1 | XM_054468194.1 |
| Pongo abelii | Phosphoenolpyruvate carboxykinase 1 | NM_001133286.1 |
| Nomascus leucogenys | Phosphoenolpyruvate carboxykinase 1 | XM_030826487.1 |
| Hylobates moloch | Phosphoenolpyruvate carboxykinase 1 | XM_032142591.2 |
| Macaca mulatta | Phosphoenolpyruvate carboxykinase 1 | XM_001086710.4 |
| Macaca nemestrina | Phosphoenolpyruvate carboxykinase 1 | XM_011722760.2 |
| Chlorocebus sabaeus | Phosphoenolpyruvate carboxykinase 1 | XM_008013224.2 |
| Mandrillus leucophaeus | Phosphoenolpyruvate carboxykinase 1 | XM_011978164.1 |
| Macaca thibetana thibetana | Phosphoenolpyruvate carboxykinase 1 | XM_050806873.1 |
| Macaca fascicularis | Phosphoenolpyruvate carboxykinase 1 | XM_005569429.3 |
| Piliocolobus tephrosceles | Phosphoenolpyruvate carboxykinase 1 | XM_023231940.2 |
| Papio anubis | Phosphoenolpyruvate carboxykinase 1 | XM_003904570.4 |
| Cercocebus atys | Phosphoenolpyruvate carboxykinase 1 | XM_012058916.1 |
| Theropithecus gelada | Phosphoenolpyruvate carboxykinase 1 | XM_025399021.1 |
| Colobus angolensis palliatus | Phosphoenolpyruvate carboxykinase 1 | XM_011936682.1 |
| Trachypithecus francoisi | Phosphoenolpyruvate carboxykinase 1 | XM_033186833.1 |
| Rhinopithecus bieti | Phosphoenolpyruvate carboxykinase 1 | XM_017852132.1 |
| Rhinopithecus roxellana | Phosphoenolpyruvate carboxykinase 1 | XM_010365119.1 |
| Marmota monax | Phosphoenolpyruvate carboxykinase 1 | XM_058575417.1 |
| Marmota monax | Phosphoenolpyruvate carboxykinase 1 | XM_058575416.1 |
| Marmota flaviventris | Phosphoenolpyruvate carboxykinase 1 | XM_027946450.1 |
| Marmota marmota marmota | Phosphoenolpyruvate carboxykinase 1 | XM_048810871.1 |
| Marmota marmota marmota | Phosphoenolpyruvate carboxykinase 1 | XM_015479925.2 |
| Aotus nancymaae | Phosphoenolpyruvate carboxykinase 1 | XM_012467511.2 |
| Sapajus apella | Phosphoenolpyruvate carboxykinase 1 | XM_032257206.1 |
| Pteronotus parnellii | Phosphoenolpyruvate carboxykinase 1 | XM_054573573.1 |
| mesoamericanus | | |
| Cebus imitator | Phosphoenolpyruvate carboxykinase 1 | XM_017536565.2 |
| Otolemur garnettii | Phosphoenolpyruvate carboxykinase 1 | XM_003787726.2 |
| Ochotona princeps | Phosphoenolpyruvate carboxykinase 1 | XM_058679364.1 |
| Ochotona princeps | Phosphoenolpyruvate carboxykinase 1 | XM_058679363.1 |
| Ochotona princeps | Phosphoenolpyruvate carboxykinase 1 | XM_004586104.3 |
| Phyllostomus hastatus | Phosphoenolpyruvate carboxykinase 1 | XM_045833174.1 |
| Callithrix jacchus | Phosphoenolpyruvate carboxykinase 1 | XM_002747710.6 |
| Mus caroli | Phosphoenolpyruvate carboxykinase 1 | XM_021149509.2 |
| Urocitellus parryii | Phosphoenolpyruvate carboxykinase 1 | XM_026406798.1 |
| Sciurus carolinensis | Phosphoenolpyruvate carboxykinase 1 | XM_047534453.1 |
| Ochotona curzoniae | Phosphoenolpyruvate carboxykinase 1 | XM_040988140.1 |
| Mus pahari | Phosphoenolpyruvate carboxykinase 1 | XM_021194801.2 |
| Desmodus rotundus | Phosphoenolpyruvate carboxykinase 1 | XM_024559406.3 |
| Mus musculus | Phosphoenolpyruvate carboxykinase 1 | NM_011044.3 |
| Mus musculus | Phosphoenolpyruvate carboxykinase 1 | AK133496.1 |
| Mus musculus | Phosphoenolpyruvate carboxykinase 1 | AK028046.1 |
| Mus musculus | Phosphoenolpyruvate carboxykinase 1 | AK030327.1 |
| Pteronotus parnellii | Phosphoenolpyruvate carboxykinase 1 | KJ957756.1 |
| Ictidomys tridecemlineatus | Phosphoenolpyruvate carboxykinase 1 | XM_013359516.3 |
| Ictidomys tridecemlineatus | Phosphoenolpyruvate carboxykinase 1 | XM_040275463.1 |
| Ictidomys tridecemlineatus | Phosphoenolpyruvate carboxykinase 1 | XM_005327077.3 |
| Mus musculus | Phosphoenolpyruvate carboxykinase 1 | BC037629.1 |
| Mus musculus | Phosphoenolpyruvate carboxykinase 1 | AK149525.1 |
| Phyllostomus discolor | Phosphoenolpyruvate carboxykinase 1 | XM_028524066.2 |
| Psammomys obesus | Phosphoenolpyruvate carboxykinase 1 | XM_055596263.1 |
| Saimiri boliviensis boliviensis | Phosphoenolpyruvate carboxykinase 1 | XM_003932619.3 |
| Cavia porcellus | Phosphoenolpyruvate carboxykinase 1 | XM_003463639.3 |
| Chinchilla lanigera | Phosphoenolpyruvate carboxykinase 1 | XM_005392250.1 |
| Meriones unguiculatus | Phosphoenolpyruvate carboxykinase 1 | XM_060382768.1 |
| Meriones unguiculatus | Phosphoenolpyruvate carboxykinase 1 | XM_021657013.2 |
| Octodon degus | Phosphoenolpyruvate carboxykinase 1 | XM_004636004.2 |
| Rattus rattus | Phosphoenolpyruvate carboxykinase 1 | XM_032904709.1 |
| Elephantulus edwardii | Phosphoenolpyruvate carboxykinase 1 | XM_006896853.1 |
| Hipposideros armiger | Phosphoenolpyruvate carboxykinase 1 | XM_019637125.1 |
| Sturnira hondurensis | Phosphoenolpyruvate carboxykinase 1 | XM_037059963.1 |
| Microcebus murinus | Phosphoenolpyruvate carboxykinase 1 | XM_012770477.2 |
| Castor canadensis | Phosphoenolpyruvate carboxykinase 1 | XM_020152656.1 |
| Peromyscus eremicus | Phosphoenolpyruvate carboxykinase 1 | XM_059259789.1 |
| Rattus norvegicus | Phosphoenolpyruvate carboxykinase 1 | BC081900.1 |
| Rattus norvegicus | Phosphoenolpyruvate carboxykinase 1 | NM_198780.3 |
| Leptonycteris yerbabuenae | Phosphoenolpyruvate carboxykinase 1 | KJ957758.1 |
| Grammomys surdaster | Phosphoenolpyruvate carboxykinase 1 | XM_028769739.1 |
| Diceros bicornis minor | Phosphoenolpyruvate carboxykinase 1 | XM_058562094.1 |
| Ceratotherium simum simum | Phosphoenolpyruvate carboxykinase 1 | XM_004430240.2 |
| Molossus molossus | Phosphoenolpyruvate carboxykinase 1 | XM_036245504.1 |
| Arvicanthis niloticus | Phosphoenolpyruvate carboxykinase 1 | XM_034495255.1 |
| Ochotona curzoniae | Phosphoenolpyruvate carboxykinase 1 | XM_040988141.1 |
| Halichoerus grypus | Phosphoenolpyruvate carboxykinase 1 | XM_036094401.1 |
| Miniopterus natalensis | Phosphoenolpyruvate carboxykinase 1 | XM_016203393.1 |
| Phocoena sinus | Phosphoenolpyruvate carboxykinase 1 | XM_032606926.1 |
| Elephantulus edwardii | Phosphoenolpyruvate carboxykinase 1 | XM_006896852.1 |
| californicus | | |
| Peromyscus californicus | Phosphoenolpyruvate carboxykinase 1 | XM_052725116.1 |
| insignis | | |
| Onychomys torridus | Phosphoenolpyruvate carboxykinase 1 | XM_036186539.1 |
| Apodemus sylvaticus | Phosphoenolpyruvate carboxykinase 1 | XM_052181590.1 |
| Carlito syrichta | Phosphoenolpyruvate carboxykinase 1 | XM_008054412.1 |
| Dipodomys spectabilis | Phosphoenolpyruvate carboxykinase 1 | XM_042699217.1 |
| Miniopterus schreibersii | Phosphoenolpyruvate carboxykinase 1 | KJ957759.1 |
| Heterocephalus glaber | Phosphoenolpyruvate carboxykinase 1 | XM_004840930.3 |
| Globicephala melas | Phosphoenolpyruvate carboxykinase 1 | XM_030841614.2 |
| Lagenorhynchus albirostris | Phosphoenolpyruvate carboxykinase 1 | XM_060123918.1 |
| Dasypus novemcinctus | Phosphoenolpyruvate carboxykinase 1 | XM_004468027.4 |
| Nycticebus coucang | Phosphoenolpyruvate carboxykinase 1 | XM_053574511.1 |
| Lemur catta | Phosphoenolpyruvate carboxykinase 1 | XM_045528475.1 |

In any of these aspects, the additional DNA constructs useful for producing an organic electrolyte can have SEQ ID NOA. 18 or SEQ ID NO. 19.

### Further Components of the Protein-Producing DNA Constructs

In one aspect, the protein-producing DNA construct has the following genetic components: a) a gene that encodes casein, b) a gene that encodes ovalbumin or a fragment thereof, c) a gene that encodes lactalbumin, d) a gene that encodes GMP.

In another aspect, said construct further includes a) a promoter, b) a terminator or stop sequence, c) a gene that confers resistance to an antibiotic (a “selective marker”), d) a reporter protein, or any combination thereof. Each of these elements is described in further detail below.

In one aspect, the construct includes from 5′ to 3′ the following genetic components in the following order: (1) a gene that encodes casein, (2) a gene that encodes ovalbumin or a fragment thereof, (3) a gene that encodes lactalbumin, (4) a gene that encodes GMP.

In one aspect, the construct includes from 5′ to 3′ the following genetic components in the following order: a gene that encodes casein having SEQ ID NO. 4 or at least 70% homology thereto, a gene that encodes an ovalbumin fragment having SEQ ID NO. 1 or at least 70% homology thereto, a gene that encodes lactalbumin having SEQ ID NO. 3 or at least 70% homology thereto, a gene that encodes GMP having SEQ ID NO. 2 or at least 70% homology thereto.

In another aspect, the construct includes from 5′ to 3′ the following genetic components in the following order: (1) a gene that encodes casein; (2) a CYC1 terminator; (3) a GAL1 promoter; (4) a gene that encodes a fragment of ovalbumin; (5) a CYC1 terminator; (6) a GAL1 promoter; (7) a gene that encodes lactalbumin; (8) a CYC1 terminator; (9) a GAL1 promoter; and (10) a gene that encodes GMP.

In another aspect, the construct includes from 5′ to 3′ the following genetic components in the following order: (1) a gene that encodes casein having SEQ ID NO. 4 or at least 90% homology thereto; (2) a CYC1 terminator; (3) a GAL1 promoter; (4) a gene that encodes a fragment of ovalbumin having SEQ ID NO. 1 or at least 90% homology thereto; (5) a CYC1 terminator; (6) a GAL1 promoter; (7) a gene that encodes lactalbumin having SEQ ID NO. 3 or at least 90% homology thereto; (8) a CYC1 terminator; (9) a GAL1 promoter; and (10) a gene that encodes GMP having SEQ ID NO. 2 or at least 90% homology thereto.

In still another aspect, the construct is a pYES2 plasmid having from 5′ to 3′ the following genetic components in the following order: (1) a gene that encodes casein having SEQ ID NO. 4 or at least 70% homology thereto; (2) a CYC1 terminator; (3) a GAL1 promoter; (4) a gene that encodes a fragment of ovalbumin having SEQ ID NO. 1 or at least 70% homology thereto; (5) a CYC1 terminator; (6) a GAL1 promoter; (7) a gene that encodes lactalbumin having SEQ ID NO. 3 or at least 70% homology thereto; (8) a CYC1 terminator; (9) a GAL1 promoter; and (10) a gene that encodes GMP having SEQ ID NO. 2 or at least 70% homology thereto.

In another aspect, the DNA construct has SEQ ID NO. 5 or at least 70% homology thereto, at least 75% homology thereto, at least 80% homology thereto, at least 85% homology thereto, at least 90% homology thereto, at least 95% homology thereto, or at least 99% homology thereto.

### Additional Components of the DNA Constructs

In another aspect, said construct further includes a) a promoter, b) a terminator or stop sequence, c) a gene that confers resistance to an antibiotic (a “selective marker”), d) a reporter protein, or any combination thereof.

In one aspect, the construct includes a regulatory sequence. In a further aspect, the regulatory sequence is already incorporated into a vector such as, for example, a plasmid, prior to genetic manipulation of the vector. In another aspect, the regulatory sequence can be incorporated into the vector through the use of restriction enzymes or any other technique known in the art.

In one aspect, the regulatory sequence is a promoter. The term “promoter” refers to a DNA sequence capable of controlling the expression of a coding sequence. In another aspect, the coding sequence to be controlled is located 3′ to the promoter. In still another aspect, the promoter is derived from a native gene. In an alternative aspect, the promoter is composed of multiple elements derived from different genes and/or promoters. A promoter can be assembled from elements found in nature, from artificial and/or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, such as, for example, in response to the presence or absence of a particular metabolite (e.g., lactose or tryptophan), a metal ion, a molecule secreted by a pathogen, or the like. A promoter also may be regulated physically, such as, for example, in response to heat, cold, water stress, salt stress, oxygen concentration, illumination, wounding, or the like.

Promoters that are useful to drive expression of the nucleotide sequences described herein are numerous and familiar to those skilled in the art. Suitable promoters include, but are not limited to, the following: T3 promoter, T7 promoter, an iron promoter, araBAD promoter, and GAL1 promoter. In a further aspect, the promoter is a native part of the vector used herein. Variants of these promoters are also contemplated. The skilled artisan will be able to use site-directed mutagenesis and/or other mutagenesis techniques to modify the promoters to promote more efficient function. The promoter may be positioned, for example, from 10-100 nucleotides from a ribosomal binding site.

In one aspect, the promoter is a GAL1 promoter. In another aspect, the GAL1 promoter is native to the plasmid used to create the vector. In another aspect, a GAL1 promoter is positioned before the gene that encodes casein, the gene that encodes ovalbumin or a fragment thereof, the gene that encodes lactalbumin, the gene that encodes GMP, or any combination thereof.

In one aspect, the regulatory sequence is an operon such as, for example, the LAC operon or LAC operator. As used herein, an “operon” is a segment of DNA containing a group of genes wherein the group is controlled by a single promoter. Genes included in an operon are all transcribed together. In a further aspect, the operon is a LAC operon and can be induced when lactose crosses the cell membrane of the biological device.

In another aspect, the regulatory sequence is a terminator or stop sequence. As used herein, a terminator is a sequence of DNA that marks the end of a gene or operon to be transcribed. In a further aspect, the terminator is an intrinsic terminator or a Rho-dependent transcription terminator. As used herein, an intrinsic terminator is a sequence wherein a hairpin structure can form in the nascent transcript that disrupts the mRNA/DNA/RNA polymerase complex. As used herein, a Rho-dependent transcription terminator requires a Rho factor protein complex to disrupt the mRNA/DNA/RNA polymerase complex. In one aspect, the terminator is an rrnB terminator obtained from or native to the pBAD plasmid. In an alternative aspect, the terminator is a CYC1 terminator obtained from or native to the pYES2 plasmid.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2000 nucleotides in the 5′ direction from the start codon of the DNA to be transcribed, or may be located 3′ to the DNA to be transcribed. Enhancers may be “cis-acting,” that is, located on the same molecule of DNA as the gene whose expression they affect.

In one aspect, and without wishing to be bound by theory, when the plasmid is a pYES2 plasmid, genes from the plasmid are ideally suited for expression in yeast under the control of the GAL1 promoter and CYC1 terminator. In a further aspect, since the expression of multiple genes is regulated under the same type of promoter, all genes arranged sequentially in a group having a 5′ GAL1 promoter and/or ending with an CYC1 terminator can be expressed together. In an alternative aspect, each gene can have its own individual GAL1 promoter situated 5′ to the gene and its own CYC1 terminator situated 3′ to the gene.

### Further Components of the DNA Constructs and Methods for Making Thereof

In another aspect, the vector contains one or more ribosomal binding sites. As used herein, a “ribosomal binding site” or “rbs” is a sequence of nucleotides located 5′ to the start codon of an mRNA that recruits a ribosome to initiate protein translation. In one aspect, the ribosomal binding site can be positioned before one or more or all genes in the DNA construct, or a before a subset of genes in a DNA construct.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for restriction enzymes. Further, restriction sites may be added, disabled, or removed as required, using techniques known in the art. In one aspect, the plasmid contains restriction sites for any known restriction enzyme such as, for example, HindIII, KpnI, SacI, BamHI, BstXI, EcoRI, BasBI, NotI, XhoI, XphI, XbaI, ApaI, SalI, ClaI, EcoRV, PstI, SmaI, XmaI, SpeI, EagI, SacII, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA at a distance of from 20 bases to over 1000 bases away from the restriction site. A variety of restriction enzymes are commercially available and their recognition sequences, as well as instructions for use (e.g., amount of DNA needed, precise volumes of reagents, purification techniques, as well as information about salt concentration, pH, optimum temperature, incubation time, and the like) are provided by enzyme manufacturers.

In one aspect, a plasmid with a polylinker containing one or more restriction sites can be digested with one restriction enzyme and a nucleotide sequence of interest can be ligated into the plasmid using a commercially-available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, usually starting just after a promoter, the order and direction of elements inserted into a plasmid can be especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleotide fragments into the plasmid.

In one aspect, different genes can be ligated into a plasmid in one pot. In this aspect, the genes will first be digested with restriction enzymes. In certain aspects, the digestion of genes with restriction enzymes provides multiple pairs of matching 5′ and 3′ overhangs that will spontaneously assemble the genes in the desired order. In another aspect, the genes and components to be incorporated into a plasmid can be assembled into a single insert sequence prior insertion into the plasmid. In a further aspect, a DNA ligase enzyme can be used to assist in the ligation process.

In another aspect, the ligation mix may be incubated in an electromagnetic chamber. In one aspect, the incubation lasts for about 1 minute, about 2 minutes, about 5 minutes, about 10 minutes, about 15 minutes, about 20 minutes, about 30 minutes, or about 1 hour.

The DNA construct described herein can be part of a vector. In general, plasmid vectors containing replicon and control sequences that are derived from species compatible with the host cell are used in connection with the hosts. The vector ordinarily carries a replication site as well as marking sequences that are capable of performing phenotypic selection in transformed cells. Plasmid vectors are well known and commercially available. Such vectors include, but are not limited to, pWLneo, pSV2cat, pOG44, pXT1, PSG, pSVK3, pBSK, pYES, pYES2, pBSKII, pET, pUC, pUC19, pBAD, and pETDuet-1 vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copier per host cell and will contain selectable markers (such as genes for antibiotic resistance) to show the skilled artisan to select host eels that have been successfully transfected with the plasmids (for example, by growing the host cells in a medium containing the antibiotic).

In one aspect, the vector encodes a selection marker. In a further aspect, the selection marker is a gene that confers resistance to an antibiotic. In certain aspects, during fermentation of host cells transformed with the vector, the cells are contacted with the antibiotic. For example, the antibiotic may be included in the culture medium. Cells that have not been successfully transformed cannot survive in the presence of the antibiotic; only cells containing the vector, which confers antibiotic resistance, can survive. Optimally, only cells containing the vector to be expressed will be cultured, as this will result in the highest production efficiency of the desired gene products (e.g., peptides). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isonizid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

In certain aspects, the DNA construct can include a gene that encodes a reporter protein. The selection of the reporter protein can vary. For example, the reporter protein can be a yellow fluorescent protein, a red fluorescent protein, a green fluorescent protein, or a cyan fluorescent protein. In one aspect, the reporter protein is an enhanced green fluorescent protein and the gene that encodes the reporter protein has SEQ ID NO. 21 or at least 70% homology thereto. In an alternative aspect, the reporter protein is a yellow fluorescent protein and the gene that encodes the reporter protein has SEQ ID NO. 20 or at least 70% homology thereto. The amount of fluorescence that is produced can be correlated to the amount of DNA incorporated into the transfected cells. The fluorescence produced can be detected and quantified using techniques known in the art. For example, spectrofluorometers are typically used to measure fluorescence. In some aspects, the additional DNA constructs useful for producing carotenoids, steviol glycosides, and/or organic electrolytes can also include genes encoding the reporter proteins described herein.

The DNA construct described herein can be part of a vector. In one aspect, the vector is a plasmid, a phagemid, a cosmid, a yeast artificial chromosome, a bacterial artificial chromosome, a virus, a phage, or a transposon.

Exemplary methods for producing the DNA constructs described herein are provided in the Examples. Restriction enzymes and purification techniques known in the art can be used to assemble the DNA constructs. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the methods described below.

### Cells and Biological Devices

A variety of different types of cells can be used in the methods described herein. In one aspect, the cells can be wild-type cells (i.e., not genetically-modified). In one aspect, the cells are from an animal such as, for example, a mammal, bird, fish, reptile, amphibian, or invertebrate. In another aspect, the cells are from a plant such as, for example, an agricultural crop, a decorative plant, a woody plant, a medicinal plant, or a combination thereof. In another aspect, the cells are from a multicellular fungus such as, for example, a mushroom, a mycorrhizal fungus, or a commercially-important mold.

In another aspect, the cells include a biological device. A “biological device” is formed when a microbial cell is transfected with a DNA construct. The biological devices are generally composed of microbial host cells, where the host cells are transformed (i.e., genetically-modified) with a DNA construct.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce an extract or composition that can remove metals and/or other contaminants from water or petroleum, such as, for example, those disclosed herein. “Heterologous” genes and proteins are genes and proteins that have been experimentally inserted into a cell that are not normally expressed by the cell. A heterologous gene may be cloned or derived from a different cell type or species than the recipient cell or organism. Heterologous genes may be introduced into cells by transduction or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes).

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the type of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed only for a limited period of time).

“Competent cells” refers to microbial cells capable of taking up heterologous DNA. Competent cells can be purchased from a commercial source, or cells can be made competent using procedures known in the art. Exemplary procedures for producing competent cells are provided in the Examples.

The host cells as referred to herein include their progeny, which are any and all subsequent generations formed by cell division. It is understood that not all progeny may be identical due to deliberate or inadvertent mutations. A host cell may be “transfected” or “transformed,” which refers to a process by which an exogenous nucleic acid is transferred or introduced into the host cell.

A transformed cell includes the primary subject cell and its progeny. The host cells can be naturally-occurring cells or “recombinant” cells. Recombinant cells are distinguishable from naturally-occurring cells in that naturally-occurring cells do not contain heterologous DNA introduced through molecular cloning procedures. In one aspect, the host cell is a prokaryotic cell such as, for example, Escherichia coli. In other aspects, the host cell is a eukaryotic cell such as, for example, the yeast Saccharomyces cerevisiae. Host cells transformed with the DNA construct described herein are referred to as “biological devices.”

The DNA construct is first delivered into the host cell. In one aspect, the host cells are naturally competent (i.e., able to take up exogenous DNA from the surrounding environment). In another aspect, cells must be treated to induce artificial competence. This delivery may be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the membranes of the cells through which the vector containing the DNA construct enters. Another method involves exposing intact yeast cells to alkali cations such as, for example, lithium. In one aspect, this method includes exposing yeast to lithium acetate, polyethylene glycol, and single-stranded DNA such as, for example, salmon sperm DNA. Without wishing to be bound by theory, the single-stranded DNA is thought to bind to the cell wall of the yeast, thereby blocking plasmids from binding. The plasmids are then free to enter the yeast cell. Enzymatic and/or electromagnetic techniques can also be used alone, or in combination with other methods, to transform microbial cells. Exemplary procedures for transforming yeast and bacteria with specific DNA constructs are provided in the Examples. In certain aspects, two or more types of DNA can be incorporated into the host cells. Thus, different metabolites can be produced from the same host cells at enhanced rates.

### Cell Culture

A satisfactory microbiological culture contains available sources of hydrogen donors and acceptors, carbon, nitrogen, sulfur, phosphorus, inorganic salts and, in certain cases, vitamins or other growth-promoting substances. For example, the addition of peptone provides a readily-available source of nitrogen and carbon. Furthermore, the use of different types of media results in different growth rates and different stationary phase densities. A rich media results in a short doubling time and higher cell density at stationary phase. Minimal media results in slow growth and low final cell densities. Efficient agitation and aeration increase final cell densities.

Culturing or fermenting of host cells can be accomplished by any technique known in the art. In one aspect, batch fermentation can be conducted. In batch fermentation, the composition of the culture medium is set at the beginning and the system is closed to future alterations. In some aspects, a limited form of batch fermentation may be carried out, wherein factors such as oxygen concentration and PH are manipulated, but additional carbon is not added. Continuous fermentation methods are also contemplated. In continuous fermentation, equal amounts of a defined medium are continuously added to and removed from a bioreactor. In other aspects, microbial cells are immobilized on a substrate. Fermentation may be carried out on any scale and may include methods in which literal “fermentation” is carried out as well as other culture methods that are non-fermentative.

In one aspect, the microorganisms can be cultured for a period of from 2 days to 2 weeks, or for about 2, about 3, about 4, about 5, about 6, about 7, about 8, about 9, about 10, about 11, about 12, about 13, or about 14 days, where any value can be the lower or upper endpoint of a range (e.g., about 3 days to about 13 days, about 8 days to about 12 days, etc.). In one aspect, the microorganisms are cultured for about 10 days.

In another aspect, the microorganisms can be cultured at any temperature appropriate for the microorganisms, with the understanding that the temperature may vary according to the microorganism (for example, a thermophilic microorganism may require a higher culture temperature than a mesophile). In one aspect, the microorganisms are cultured at a temperature of from about 20 to about 37° C., or are cultured at about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C., where any value can be the lower or upper endpoint of a range, where any value can be the lower or upper endpoint of a range (e.g., about 21° C. to about 36° C., about 25° C. to about 30° C., etc.).

In certain aspects, after culturing the microorganisms for a sufficient time, the microbial cells can be lysed with one or more enzymes. For example, when the microbial cells are fungal, the fungal cells can be lysed with lyticase. In one aspect, the lyticase concentration can be about 500 μL, about 600 μL, about 700 μL, about 800 μL, about 900 μL, or about 1,000 μL per liter of culture, where any value can be the lower or upper endpoint of a range, where any value can be the lower or upper endpoint of a range (e.g., about 500 μL to about 900 μL, about 600 μL to about 800 μL, etc.).

In addition to or in place of enzymes, other components can be used to facilitate lysis of the microbial cells. In one aspect, chitosan can be used in combination with an enzyme to lyse the microbial cells. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation. In one aspect, the chitosan is from about 60% to about 100% acetylated, or about 70%, about 75%, about 80%, about 85%, about 90%, about 95%, or about 100% acetylated, where any value can be the lower or upper endpoint of a range, where any value can be the lower or upper endpoint of a range (e.g., about 60% to about 90%, about 70% to about 80%, etc.).

The molecular weight of the chitosan can vary, as well. For example, the chitosan can comprise about 3, 4, 5, 6, 7, 8, 9, 10, 11, 12, 13, 14, 15, 16, 17, 18, 19, or 20 glucosamine units and/or N-acetylglucosamine units, where any value can be the lower or upper endpoint of a range, where any value can be the lower or upper endpoint of a range (e.g., 2 to 19, 3 to 10, 5 to 7, etc.). In one aspect, chitosan can be added until a concentration of about 0.0015%, about 0.0025%, about 0.005%, about 0.0075%, about 0.01%, about 0.015%, about 0.02%, about 0.03%, about 0.04%, or about 0.05%, where any value can be an upper or lower endpoint of a range (e.g., 0.002% to 0.04%, 0.05% to 0.015%, etc.).

In another aspect, cells can first be fermented, for example, in a biofermenter, at a temperature conducive to cell growth. In one aspect, the cells are fermented at 30° C. In a further aspect, the cells are fermented for a time period sufficient to produce the metabolite(s) of interest. In one aspect, the cells are fermented for from 6 hours to 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, during fermentation, a micro-current can be applied to the cells as described above. In some aspects, the micro-current is applied for the entire culture period. In another aspect, the micro-current is applied for only a part of the culture period, or for several non-consecutive parts of the culture period. In one aspect, the micro-current is the same throughout the entire culture period. In an alternative aspect, the micro-current is varied during the culture period.

Exemplary methods for culturing cells and/or the biological devices disclosed herein are provided in the Examples.

### Culture Medium

In some aspects, the cells are suspended in a culture medium. In another aspect, the culture medium can be Dulbecco's Modified Eagle Medium (DMEM), RPMI 1640, Minimal Essential Medium (MEM), Eagle's Minimal Essential Medium (EMEM), Iscove's Modified Dulbecco's Medium (IMDM), DMEM/F12 Medium, Murashige and Skoog (MS) medium, White's medium, Agrobacterium minimal medium, Banana AGS basal medium, Blaydes basal medium, Bold's basal medium, Chu (N6) medium, De Greef and Jacobs Medium, DKW basal medium, Economou and Read basal medium, Gamborg (B5) medium, Gresshoff and Doy medium, Heller medium, Hoagland complete medium, Jensen's medium, Kao and Michayluk medium, Litvay medium, NB basal medium, Nitsch medium. NLN medium, Quoirin and Lepoivre medium, Schenk and Hildebrandt medium, TAP medium, TM4G medium, Vacin and Went medium, wheat callus induction medium, Luria Bertani (LB) broth, terrific broth, tryptic soy broth, minimal salts (M9) medium, SOB medium, SOC medium, yeast malt medium, YPD broth, YNB broth, synthetic complete (SC) medium, YPG medium, Hartwell's complete (HC) medium, or a combination thereof. In one aspect, the culture medium is Luria Bertani (LB) broth or yeast malt medium.

In another aspect, the culture medium can contain supplemental compounds such as, for example, vitamins, nucleosides, nucleotides, amino acids, a carbohydrate, an antibiotic, or a combination thereof.

In one aspect, the culture medium can be a liquid. In another aspect, the methods disclosed herein can be performed in a biofermenter. In an alternative aspect, the cells can be distributed on a substrate. In one aspect, the substrate can be agar, a culture dish, contaminated soil, a wastewater treatment device, mineral ore, a plant organ, a tissue scaffold, or a fermentable material. When the substrate is a plant organ, in some aspects, the plant organ can be a root, leaf, stem, rhizome, tuber, flower, seed, fruit, vegetable, callus, or a combination thereof. When the substrate is a fermentable material, in some aspects, the substrate can be milk, a grain, cabbage, soybeans, fish, or a biomass feedstock. When the substrate is a biomass feedstock, in some aspects, the substrate can be forestry residue, logging residue, sawmill residue, animal manure, a recycled material, a carbohydrate waste, corn cob, corn stover, wheat straw, nut hulls, soy hulls, switchgrass, gammagrass, paper, or a combination thereof.

### Extraction and Purification of Metabolites

In one aspect, the methods disclosed herein can be used to increase the production of metabolites by cells. In some aspects, the metabolites are secreted into a culture medium and collected. In other aspects, the metabolites remain in the cells, requiring the cells to be lysed prior to collection and purification of the metabolites. In one aspect, the metabolites are the peptides, proteins, and fragments thereof encoded by the genes of the disclosed DNA constructs (e.g. ovalbumin, GMP, lactalbumin, and casein).

In one aspect, prior to collection of any metabolite(s) of interest, fermentation can be stopped. In some aspects, the micro-current will be withdrawn or turned off (e.g., by turning off a power supply to a biofermenter or a similar mechanism). In another aspect, an enzyme such as, for example, lyticase can optionally be used to lyse cells following fermentation. In still another aspect, the cell culture can optionally be autoclaved for a sufficient time following cell lysis in order to ensure no living cells remain in the culture. Following lysis and autoclaving, or instead of performing these two processes, centrifugation, sonication, and filtration can be performed to facilitate collection of relevant metabolites. In an alternative aspect, culture medium including an increased concentration of the desired metabolite(s) from the biofermenter can be used without further processing.

### Use of Micro-Current to Enhance Cell Culture

As used herein, “micro-current” refers to an electric current of from about 50 mV to about 300 mV. In one aspect, when a micro-current is applied to cells (e.g., a substrate on which the cells are growing or a culture medium in which the cells are growing) via one or more electrodes, cell growth and division and/or the production of one or more desirable metabolites from the cells is increased.

“Growth” as used herein refers to an increase in size or population of cells, or both, whereas “increase” used with respect to a treated population of cells (e.g., exposed to micro-current) refers to the treated cells having larger size and/or to the presence of a higher number of cells or colony forming units (CFUs) compared to a reference population of identical but untreated cells (i.e., no exposure to the micro-current) after a predetermined period of time.

“Metabolites” are any substances produced during metabolism. In some aspects, metabolites are essential to cellular function (e.g., glycolysis intermediates) or cell signaling, or can be produced by organisms in times of stress (e.g., heat shock proteins), or are compounds produced by plants or microorganisms that have been found to have functions useful in industry and/or medicine (e.g., vitamins, antibiotics, sweeteners, or the like). Metabolites can be purified (e.g., lycopene and other carotenoids) or can be used as crude extracts (e.g., the organic electrolytes and anti-microbial compounds disclosed herein). In any of these aspects, exposure to a micro-current causes treated cells to increase production of one or more metabolites.

“Production” as used herein refers to the making of one or more metabolites by cells. In one aspect, the metabolites are secreted by the cells into a culture medium. In an alternative aspect, the metabolites remain in the cell interiors and the cells must be lysed to release the metabolites. Metabolite production can be accomplished via one of the following means: (1) for peptide and protein metabolites, transcription of DNA to RNA and translation of RNA to a protein; (2) for non-peptide metabolites, transcription and translation of one or more proteins followed by protein-based catalysis of reactions that transform one or more precursor molecules (e.g., lipids, sugars, amino acids, nucleotides and nucleotide components, and other small molecules) into the desired metabolite; and (3) other methods known in the art but not already listed. In one aspect, exposure to a micro-current as disclosed herein can increase the production of one or more metabolites by cells.

In one aspect, disclosed herein is a method for growing cells, wherein the method includes exposing the cells to a micro-current during the growth of the cells. In another aspect, exposing the cells to the micro-current increases cell population as compared to identical cells that are not exposed to the micro-current.

### Micro-Current

In one aspect, a micro-current is applied to the cells and/or biological devices disclosed herein. In one aspect, the micro-current is from about 50 to about 300 mV, or is about 50, 75, 100, 120, 125, 150, 175, 200, 220, 225, 250, 275, or about 300 mV, or a combination of any of the foregoing values, or a range encompassing any of the foregoing values. In one aspect, the micro-current is about 120 mV. In another aspect, the micro-current is about 200 mV. In an aspect, regardless of the micro-current voltage, metabolite production and cellular growth are higher than for otherwise identical cells in the absence of a micro-current. However, in one aspect, a higher micro-current (e.g. 200 mV) causes colony size to increase compared to a lower (e.g. 120 mV) micro-current. In another aspect, lower micro-current (e.g. 120 mV) may result in a larger number of colonies but with each individual colony having a smaller size than with a higher micro-current (e.g. 200 mV). In any of these aspects, then, the desired outcome (size of colonies, colony count, or both) can be tailored to a specific project.

In one aspect, the cells are exposed to the micro-current using at least one electrode. In a further aspect, the electrode is connected to a power supply. In some aspects, the power supply can be adjusted to provide variable voltages for the micro-current. In an alternative aspect, the electrode can be connected to a battery. In any of these aspects, the at least one electrode can be made from copper, graphite, carbon nanotubes, graphene, titanium, brass, silver, platinum, palladium, iron, nickel, lead, steel, magnesium, aluminum, tin, zinc, tungsten, mixed metal oxides, a spinel-type structure, an olivine-type structure, or a combination thereof. In some aspects, the at least one electrode is made from platinum. In another aspect, if more than one electrode is used in the processes disclosed herein, the electrodes can be made from two different materials (e.g., graphene and platinum).

In another aspect, the cells and/or biological devices disclosed herein are exposed to a micro-current for from about 6 hours to about 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, the cells and/or biological devices disclosed herein are exposed to a micro-current for about 48 hours.

In another aspect, cells can first be fermented, for example, in a biofermenter, at a temperature conducive to cell growth. In one aspect, the cells are fermented at 30° C. In a further aspect, the cells are fermented for a time period sufficient to produce the metabolite(s) of interest. In one aspect, the cells are fermented for from 6 hours to 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, during fermentation, a micro-current can be applied to the cells as described above. In some aspects, the micro-current is applied for the entire culture period. In another aspect, the micro-current is applied for only a part of the culture period, or for several non-consecutive parts of the culture period. In one aspect, the micro-current is the same throughout the entire culture period. In an alternative aspect, the micro-current is varied during the culture period.

Exemplary methods for culturing cells and/or the biological devices disclosed herein are provided in the Examples.

In some aspects, the cells are suspended in a culture medium. In another aspect, the culture medium can be Dulbecco's Modified Eagle Medium (DMEM), RPMI 1640, Minimal Essential Medium (MEM), Eagle's Minimal Essential Medium (EMEM), Iscove's Modified Dulbecco's Medium (IMDM), DMEM/F12 Medium, Murashige and Skoog (MS) medium, White's medium, Agrobacterium minimal medium, Banana AGS basal medium, Blaydes basal medium, Bold's basal medium, Chu (N6) medium, De Greef and Jacobs Medium, DKW basal medium, Economou and Read basal medium, Gamborg (B5) medium, Gresshoff and Doy medium, Heller medium, Hoagland complete medium, Jensen's medium, Kao and Michayluk medium, Litvay medium, NB basal medium, Nitsch medium. NLN medium, Quoirin and Lepoivre medium, Schenk and Hildebrandt medium, TAP medium, TM4G medium, Vacin and Went medium, wheat callus induction medium, Luria Bertani (LB) broth, terrific broth, tryptic soy broth, minimal salts (M9) medium, SOB medium, SOC medium, yeast malt medium, YPD broth, YNB broth, synthetic complete (SC) medium, YPG medium, Hartwell's complete (HC) medium, or a combination thereof. In one aspect, the culture medium is Luria Bertani (LB) broth or yeast malt medium.

In another aspect, the culture medium can contain supplemental compounds such as, for example, vitamins, nucleosides, nucleotides, amino acids, a carbohydrate, an antibiotic, or a combination thereof.

In one aspect, the culture medium can be a liquid. In another aspect, the methods disclosed herein can be performed in a biofermenter. In an alternative aspect, the cells can be distributed on a substrate. In one aspect, the substrate can be agar, a culture dish, contaminated soil, a wastewater treatment device, mineral ore, a plant organ, a tissue scaffold, or a fermentable material. When the substrate is a plant organ, in some aspects, the plant organ can be a root, leaf, stem, rhizome, tuber, flower, seed, fruit, vegetable, callus, or a combination thereof. When the substrate is a fermentable material, in some aspects, the substrate can be milk, a grain, cabbage, soybeans, fish, or a biomass feedstock. When the substrate is a biomass feedstock, in some aspects, the substrate can be forestry residue, logging residue, sawmill residue, animal manure, a recycled material, a carbohydrate waste, corn cob, corn stover, wheat straw, nut hulls, soy hulls, switchgrass, gammagrass, paper, or a combination thereof.

In one aspect, when the cells to be exposed to a micro-current are in a liquid medium (e.g., in a biofermenter), the at least one electrode can be immersed in the liquid medium. In an alternative aspect, the biofermenter can have one or more panels, elements, or contact points built into the biofermenter walls, wherein the panels, elements, or contact points are made from electrode materials as disclosed herein, and wherein the panels, elements, or contact points come into contact with the liquid culture medium.

In still another aspect, when the cells to be exposed to a micro-current are in contact with a substrate, the manner of contact with the at least one electrode with the substrate will depend on the characteristics of the substrate. In a further aspect, if the substrate is a solid material such as, for example, a component of a wastewater treatment device or a tissue scaffold, the substrate can include a conductive material such as, for example, copper, graphite, carbon nanotubes, graphene, titanium, brass, silver, platinum, palladium, iron, nickel, lead, steel, magnesium, aluminum, tin, zinc, tungsten, mixed metal oxides, a spinel-type structure, an olivine-type structure, or a combination thereof. In a further aspect, the conductive material can be present as a wire or network of wires, as a coating, as the substrate itself, or any other form useful for and conducive to the passage of micro-current through the cells. In an alternative aspect, an electrolyte solution or gel can be applied to the substrate in order to facilitate the passage of the micro-current through the cells. In any of these aspects, the cells can be applied to the substrate using any means known in the art and the at least one electrode can be placed into direct contact with the substrate, conductive material, or electrolyte in order to apply the micro-current to the cells.

In one aspect, when the substrate is a material intended for further processing or industrial use (e.g., a fermentable material, contaminated soil, a mineral ore, or the like), the substrate can optionally be milled, crushed, and/or ground to reduce the particle size (as in a mineral ore) to increase the surface area of the substrate. In a further aspect, the substrate can optionally be contacted with a liquid suitable for sustaining cellular life and growth along with the cells intended to perform the processing (e.g., cells to ferment the material, to decontaminate the soil, to extract the mineral from the ore, or another function). Further in this aspect, the liquid can be capable of conducting electricity. In still another aspect, following processing as described herein, the substrate can be contacted with the at least one electrode by a means such as, for example, immersing or burying the electrode in the substrate.

In any of the above aspects, the cells can be genetically modified. In an alternative aspect, the cells are not genetically-modified. In some aspects, the cells can include both genetically-modified and non-genetically-modified cells.

In one aspect, application of a micro-current as disclosed herein increases cell growth. In a further aspect, increased cell growth can be evaluated by culturing identical cells in two groups, wherein one group is exposed to a micro-current and the other group is not exposed to a micro-current. After a given culture period, the group exposed to the micro-current according to the methods disclosed herein will contain a greater number of colony forming units (CFUs) compared to identical cells that were not exposed to a micro-current. In another aspect, the group exposed to the micro-current according to the method disclosed herein will, if capable of producing a metabolite, have produced a greater amount of the metabolite compared to identical cells that were not exposed to a micro-current. In still another aspect, the group exposed to the micro-current according to the method disclosed herein will contain larger cells compared to identical cells that were not exposed to the micro-current. In some aspects, cells exposed to a micro-current will display a combination of characteristics (e.g., both increased metabolite production and increased cell size, or increased metabolite production and a greater number of CFUs, or some combination thereof). In another aspect, different cell types (e.g., E. coli and S. cerevisiae) with different modifications (e.g., wild type, modified with a DNA construct as disclosed herein, etc.) will respond to different levels of micro-current with different growth increases.

In one aspect, cells exposed to a micro-current will display from greater than 1 to about 5 times more CFUs than identical cells not exposed to a micro-current, or about 1.1, 1.5, 2, 2.5, 3, 3.5, 4, 4.5, or about 5 times more CFUs, or a combination of any of the foregoing values, or a range encompassing any of the foregoing values. In another aspect, cells exposed to a micro-current will display about 1.1, 1.16, 1.27, 1.38, 1.41, or about 1.5 times more CFUs than identical cells not exposed to a micro-current.

In one aspect, S. cerevisiae cells containing DNA constructs capable of producing an organic electrolyte, a carotenoid, or complete proteins as disclosed herein form more CFUs when exposed to a 120 mV micro-current. In another aspect, S. cerevisiae cells containing DNA constructs capable of producing a polyactive carbohydrate form more CFUs when exposed to a 200 mV micro-current.

### Metabolite Production

In one aspect, metabolite production increases when cells capable of producing the metabolite are exposed to a micro-current. In a further aspect, when cells capable of producing complete proteins, carotenoids, steviol glycosides, and/or organic electrolytes as disclosed herein are contacted with a micro-current, the cells produce from greater than 1 to about 5 times more of these metabolites than identical cells not contacted with a micro-current, or from about 1.05, 1.1, 1.5, 2, 2.5, 3, 3.5, 4, 4.5, or about 5 times more of the specified metabolites, or a combination of any of the foregoing values, or a range encompassing any of the foregoing values.

### Oral Dosage Forms

In various aspects, the present disclosure relates to nutritional compositions comprising a at least one product of a disclosed method (e.g. an extract or lysate from a biological device as described herein). In some aspects, the nutritional compositions can include pharmaceutically acceptable carriers. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed nutritional compositions can be conveniently presented in unit dosage form and prepared by any of the methods well known in the art of pharmacy and pharmaceutical sciences.

In a further aspect, the disclosed nutritional compositions comprise at least one disclosed extract or lysate and/or at least one product of a disclosed method and a pharmaceutically acceptable carrier. The disclosed nutritional compositions include those suitable for oral administration. In various aspects, the present disclosure also relates to a nutritional composition comprising a pharmaceutically acceptable carrier or diluent.

In practice, the compositions of the present disclosure can be combined as the active ingredient in intimate admixture with a pharmaceutical carrier according to conventional pharmaceutical compounding techniques. Thus, the nutritional compositions of the present disclosure can be presented as discrete units suitable for oral administration such as capsules, cachets or tablets each containing a predetermined amount of the nutritional composition. Further, the compositions can be presented as a powder, as granules, as a solution, as a suspension in an aqueous liquid, as a non-aqueous liquid, as an oil-in-water emulsion or as a water-in-oil liquid emulsion.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the composition to the patient can open a single container or package with the entire dose contained therein, and does not have to mix any components together from two or more containers or packages. Typical examples of unit dosage forms are tablets (including scored or coated tablets), capsules or pills for oral administration; powder packets; wafers; and segregated multiples thereof. This list of unit dosage forms is not intended to be limiting in any way, but merely to represent typical examples of unit dosage forms.

The pharmaceutical compositions disclosed herein comprise a nutritional composition of the present disclosure as an active ingredient, a pharmaceutically acceptable carrier, and optionally one or more additional vitamins or similar agents.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical or food-grade diluents, excipients, extenders, or carriers (termed herein as a pharmaceutically acceptable carrier, or a carrier) suitably selected with respect to the intended form of administration and as consistent with conventional pharmaceutical practices. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent advantageous oral dosage unit forms in which case solid pharmaceutical carriers are obviously employed. However, other dosage forms may be suitable depending upon clinical population (e.g., age and severity of clinical condition), solubility properties of the specific disclosed composition used, and the like. Accordingly, the disclosed compounds can be used in oral dosage forms such as pills, powders, granules, elixirs, tinctures, suspensions, syrups, sublingual dissolvable strips, hydrogel microparticles, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example Eudragit® RS), copolymerizates of acrylic and methacrylic acid esters and trimethyl ammonium methacrylate (for example Eudragit® RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N, N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl) imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In some aspects, the oral dosage form can be a rapidly dissolving film (RDF) or oral thin film (OTF) to be placed in an area of the mouth such as the sublingual area or the buccal cavity. In a further aspect, such films can include strip-forming polymers, plasticizers, sweetening and/or flavoring agents, coloring agents, stabilizers, thickeners, permeation enhancers, disintegrants, saliva stimulating agents, and the like. In a further aspect, these ingredients are already approved for use in other oral pharmaceutical dosage forms such as those described above. In other aspects, the oral dosage form can be a fast-dissolving tablet or lozenge or the like. In one aspect, the strip-forming polymers can be hydrophilic polymers and can be prepared into strip form by solvent casting and/or hot-melt extrusion. In one aspect, RDF or OTF formulations can have a large surface area for fast disintegration and absorption of active ingredients. In a still further aspect, the film formulations may adhere to the oral cavity by a method such as, for example, interaction with saliva, such that the film does not become displaced in the oral cavity and/or accidentally swallowed. In an aspect, the stabilizer can be ethanol, n-propanol, glycerol, a polyethylene glycols with a molecular weights between 200 Da and 600 Da, diethylene glycol monoethyl ether, 1,2-propylene glycol, urea, urethane, acetamide, N-methyl acetamide, N,N-diethyl acetamide, N,N-dimethyl acetamide, ethylenediamine, hydroxyethyl theophylline, tromethamine, an aliphatic amino acid, or any combination thereof. In some aspects, a component can act in more than one capacity (e.g. propylene glycol can be a stabilizer, a permeation enhancer, a plasticizer, or any combination thereof.

Exemplary components of oral thin films include, but are not limited to, the following. In one aspect, the water soluble strip-forming polymer can be selected from hydroxypropyl methylcellulose (HPMC) E3, HPMC E5, HPMC E15, HPMC K-3, methylcellulose A-3, methylcellulose A-6, methylcellulose A-15, pullulan, carboxmethylcellulose or a derivative thereof, polyvinylpyrollidone (PVP) K-90, pectin, gelatin. sodium alginate, hydroxypropylcellulose, polyvinyl alcohol, maltodextrins, calcium alginate, a polyactive carbohydrate, chitosan, or any combination thereof. In one aspect, polyactive carbohydrates are further described in U.S. Pat. Nos. 10,995,353 and 12,129,500. In some aspects, strip-forming polymers can also serve as disintegrants, or a separate disintegrant can be added, such as, for example, cross-linked polyvinyl pyrrolidone, sodium carboxymethyl starch, sodium carboxymethyl cellulose, microcrystalline cellulose, or any combination thereof. In one aspect, suitable permeation enhancers include, but are not limited to, 2,3-lauryl ether, aprotinin, azone, benzalkonium chloride, cetylpyridinium chloride, cetyltrimethyl ammonium bromide, cyclodextrin, dextran sulfate, lauric acid, lysophosphatidylcholine, menthol, phosphatidylcholine, polyoxyethylene, polysorbate 80, propylene glycol, disodium ethylenediaminetetraacetic acid (EDTA), sodium glycocholate, sodium glycodeoxycholate, sodium lauryl sulfate, sodium salicylate, sodium taurocholate, sodium taurodeoxycholate, or any combination thereof. In another aspect, suitable plasticizers include glycerol, dibutyl phthalate, polyethylene glycol, and combinations thereof. In still another aspect, useful surfactants include sodium lauryl sulfate, benzalkonium chloride, polysorbates, and combinations thereof, while useful sweeteners include saccharin, cyclamate, aspartame, steviol glycosides, or combinations thereof. In a still further aspect, saliva stimulating agents, which may also enhance flavor, include, but are not limited to, citric acid, malic acid, lactic acid, ascorbic acid, and combinations thereof. In an aspect, ascorbic acid may be preferred since it is a useful vitamin. (i.e., vitamin C) in nutritional compositions. In some aspects, for oral thin films and/or rapidly dissolving films, the following general amounts of components may be used: active ingredient or lysate/extract: 5-30% by weight, polymer: 45% by weight, plasticizer: 0-20% by weight, sweetener: 3-6% by weight, and saliva stimulating agent: 2-6% by weight. However, amounts of components can be varied depending on the formulation and desired effect and should thus also be considered disclosed.

In another aspect, the nutritional supplements and oral dosage forms can be provided as hydrogel microparticles. In a further aspect, the hydrogel microparticles can include one or more hydrogel forming materials such as, for example, sodium alginate, calcium alginate, a polyactive carbohydrate, chitosan, glucosamine, chondroitin, or any combination thereof. In one embodiment, the hydrogel microparticles can include chitosan (1% w/v) in acetic acid 3% (w/v) and sodium alginate (2% w/v). Further in this aspect, to produce the hydrogels, one or more of sodium hydroxide (NaOH) and/or calcium chloride (CaCl₂)) can be added to the hydrogel forming materials. In an aspect, the hydrogel microparticles can encapsulate the active compounds (proteins, vitamins, and the like) from the disclosed lysates and extracts and may facilitate absorption of the compounds, delivery of the compounds, or the like.

In some aspects, any of the above-listed oral dosage forms can be formulated for addition to food or beverage products, either by incorporating the dosage forms into the food or beverage products at the time of manufacture, or being added by the consumer to the food or beverage products. In a further aspect, the disclosed lysates and extracts can be provided as freeze-dried, spray-dried, or other powders for incorporation into foods and beverages by the consumer or in manufacturing facilities.

### Advantages and Applications of the Disclosed Formulations and Compositions

In an aspect, the disclosed formulations and compositions can provide supplemental nutrition to those in need thereof for a variety of medical reasons. In one aspect, patients with neurological or other conditions that impair swallowing or otherwise cause dysphagia (e.g., stroke, amyotrophic lateral sclerosis, and/or Parkinson's disease), forms of supplemental nutrition capable of being sublingually absorbed may be particularly important. In another aspect, individuals with gastrointestinal disease or reduced stomach capacity due to bariatric surgery may benefit from additional proteins and vitamins in non-bulky or low-volume forms. In some aspects, cancer patients and others experiencing nausea may benefit from nutrition sources that can be absorbed sublingually. In an additional aspect, some individuals with dietary restrictions such as vegans may wish for complete protein sources without consuming products produced by animals, and some athletes may wish for additional protein for performance purposes, with or without consuming bulky foods and large volumes of liquid. In one aspect, the present compositions and formulations can address each of these needs. In some aspects, however, when the consumer is able to consume bulk foods and beverages but in need of supplemental nutrition, the disclosed compositions can be added to the foods and/or beverages that are already part of the consumer's diet.

In an aspect, one advantage of a sublingual strip or film is that it can be consumed without additional liquid, such as, for example, would be required for swallowing a tablet or capsule. In another aspect, sublingual strips may be advantageous because components thereof can be directly absorbed into the bloodstream. In a further aspect, direct absorption can bypass the gastrointestinal tract and avoid digestion, swallowing or choking difficulties, vomiting or nausea, and the like. In alternative aspects, the strips can be dissolved in the buccal cavity instead of sublingually.

### ASPECTS

The present disclosure can be described in accordance with the following numbered aspects, which should not be confused with the claims.

Aspect 1. A DNA construct comprising the following genetic components:
(a) a gene that encodes casein;
(b) a gene that encodes ovalbumin or a fragment thereof;
(c) a gene that encodes lactalbumin; and
(d) a gene that encodes glycomacropeptide (GMP).

Aspect 2. The DNA construct of aspect 1, wherein the gene that encodes the casein has SEQ ID NO. 4 or at least 70% homology thereto.

Aspect 3. The DNA construct of aspect 1 or 2, wherein the gene that encodes the fragment of ovalbumin has SEQ ID NO. 1 or at least 70% homology thereto.

Aspect 4. The DNA construct of any one of aspects 1-3, wherein the gene that encodes lactalbumin has SEQ ID NO. 3 or at least 70% homology thereto.

Aspect 5. The DNA construct of any one of aspects 1-4, wherein the gene that encodes GMP has SEQ ID NO. 2 or at least 70% homology thereto.

Aspect 6. The DNA construct of any one of aspects 1-5, wherein the construct further comprises at least one promoter.

Aspect 7. The DNA construct of aspect 6, wherein the at least one promoter is a T3 promoter, a T7 promoter, an iron promoter, an araBAD promoter, a GAL1 promoter, or any combination thereof.

Aspect 8. The DNA construct of aspect 7, wherein the at least one promoter is GAL1 promoter, and the GAL1 promoter is positioned before the gene that encodes casein, the gene that encodes ovalbumin or a fragment thereof, the gene that encodes lactalbumin, the gene that encodes GMP, or any combination thereof.

Aspect 9. The DNA construct of any one of aspects 1-8, wherein the DNA construct further comprises a gene that confers resistance to an antibiotic.

Aspect 10. The DNA construct of aspect 9, wherein the antibiotic comprises tetracycline, neomycin, kanamycin, ampicillin, hygromycin, chloramphenicol, amphotericin B, bacitracin, carbapenem, cephalosporin, ethambutol, fluoroquinolones, isoniazid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

Aspect 11. The DNA construct of any one of aspects 1-10, wherein the DNA construct further comprises at least one terminator.

Aspect 12. The DNA construct of aspect 11, wherein the at least one terminator is an CYC1 terminator.

Aspect 13. The DNA construct of any one of aspects 1-12, wherein the construct comprises from 5′ to 3′ the following genetic components in the following order: (a) a gene that encodes casein; (b) a gene that encodes ovalbumin or a fragment thereof; (c) a gene that encodes lactalbumin; and (d) a gene that encodes GMP.

Aspect 14. The DNA construct of any one of aspects 1-13, wherein the construct comprises from 5′ to 3′ the following genetic components in the following order: (a) a gene that encodes a casein having SEQ ID NO. 4 or at least 70% homology thereto; (b) a gene that encodes a fragment of ovalbumin having SEQ ID NO. 1 or at least 70% homology thereto; (c) a gene that encodes lactalbumin having SEQ ID NO. 3 or at least 70% homology thereto; and (d) a gene that encodes GMP having SEQ ID NO. 2 or at least 70% homology thereto.

Aspect 15. The DNA construct of any one of aspects 1-14, wherein the construct comprises from 5′ to 3′ the following genetic components in the following order: (a) a gene that encodes casein, (b) a CYC1 terminator, (c) a GAL1 promoter, (d) a gene that encodes a fragment of ovalbumin, (e) a CYC1 terminator, (f) a GAL1 promoter, (g) a gene that encodes lactalbumin, (h) a CYC1 terminator, (i) a GAL1 promoter, and (j) a gene that encodes GMP.

Aspect 16. The DNA construct of any one of aspects 1-15, wherein the construct comprises from 5′ to 3′ the following genetic components in the following order: (a) a gene that encodes casein having SEQ ID NO. 4 or at least 70% homology thereto, (b) a CYC1 terminator, (c) a GAL1 promoter, (d) a gene that encodes a fragment of ovalbumin having SEQ ID NO. 1 or at least 70% homology thereto, (e) a CYC1 terminator, (f) a GAL1 promoter, (g) a gene that encodes lactalbumin having SEQ ID NO. 3 or at least 70% homology thereto, (h) a CYC1 terminator, (i) a GAL1 promoter, and (j) a gene that encodes GMP having SEQ ID NO. 2 or at least 70% homology thereto.

Aspect 17. The DNA construct of any one of aspects 1-16, wherein the DNA construct has SEQ ID NO. 5.

Aspect 18. A vector comprising the DNA construct of any one of aspects 1-17.

Aspect 19. The vector of aspect 18, wherein the vector is a plasmid.

Aspect 20. The vector of aspect 19, wherein the plasmid is pWLneo, pSV2cat, pOG44, pXT1, pSG, pSVK3, pBSK, pBSKII, pYES, pYES2, pET, pBAD, pUC, or pUC19.

Aspect 21. The vector of aspect 19, wherein the plasmid is pYES2.

Aspect 22. A biological device comprising host cells transformed with the DNA construct in any one of aspects 1-21.

Aspect 23. The biological device of aspect 22, wherein the host cells comprise fungi.

Aspect 24. The biological device of aspect 23, wherein the fungi comprise Saccharomyces cerevisiae.

Aspect 25. An extract produced by culturing the biological device of any one of aspects 22-24 in a culture medium, wherein the extract comprises casein, ovalbumin or a fragment thereof, lactalbumin, and GMP.

Aspect 26. The extract of aspect 25, wherein the host cells are lysed to produce a lysate.

Aspect 27. The extract of aspect 25 or 26, wherein the cells are exposed to a micro-current during the culturing of the cells.

Aspect 28. The extract of any one of aspects 25-27, wherein the culture medium comprises Dulbecco's Modified Eagle Medium (DMEM), RPMI 1640, Minimal Essential Medium (MEM), Eagle's Minimal Essential Medium (EMEM), Iscove's Modified Dulbecco's Medium (IMDM), DMEM/F12 Medium, Murashige and Skoog (MS) medium, White's medium, Agrobacterium minimal medium, Banana AGS basal medium, Blaydes basal medium, Bold's basal medium, Chu (N6) medium, De Greef and Jacobs Medium, DKW basal medium, Economou and Read basal medium, Gamborg (B5) medium, Gresshoff and Doy medium, Heller medium, Hoagland complete medium, Jensen's medium, Kao and Michayluk medium, Litvay medium, NB basal medium, Nitsch medium. NLN medium, Quoirin and Lepoivre medium, Schenk and Hildebrandt medium, TAP medium, TM4G medium, Vacin and Went medium, wheat callus induction medium, Luria Bertani (LB) broth, terrific broth, tryptic soy broth, minimal salts (M9) medium, SOB medium, SOC medium, yeast malt medium, YPD broth, YNB broth, synthetic complete (SC) medium, YPG medium, Hartwell's complete (HC) medium, or a combination thereof.

Aspect 29. The extract of any one of aspects 25-27, wherein the culture medium comprises Luria Bertani (LB) broth or yeast malt medium.

Aspect 30. The extract of any one of aspects 25-29, wherein the culture medium comprises supplemental vitamins, nucleosides, nucleotides, amino acids, a carbohydrate, an antibiotic, or a combination thereof.

Aspect 31. The extract of any one of aspects 25-30, wherein the culture medium comprises a liquid.

Aspect 32. The extract of aspect 31, wherein the host cells are cultured in a biofermenter.

Aspect 33. The extract of any one of aspects 25-30, wherein the host cells are distributed on a substrate.

Aspect 34. The extract of aspect 33, wherein the substrate comprises agar.

Aspect 35. The extract of any one of aspects 27-34, wherein the host cells are exposed to the micro-current using at least one electrode.

Aspect 36. The extract of aspect 35, wherein the at least one electrode comprises copper, graphite, carbon nanotubes, graphene, titanium, brass, silver, platinum, palladium, iron, nickel, lead, steel, magnesium, aluminum, tin, zinc, tungsten, mixed metal oxides, a spinel-type structure, an olivine-type structure, or a combination thereof.

Aspect 37. The extract of aspect 35, wherein the at least one electrode comprises platinum.

Aspect 38. The extract of any one of aspects 27-37, wherein the micro-current is from about 50 mV to about 300 mV.

Aspect 39. The extract of any one of aspects 27-37, wherein the micro-current is about 120 mV.

Aspect 40. The extract of any one of aspects 27-37, wherein the micro-current is about 200 mV.

Aspect 41. The extract of any one of aspects 27-40, wherein cell growth is greater than 1 to about 5 times higher for the host cells exposed to the micro-current compared to otherwise identical cells not exposed to the micro-current.

Aspect 42. The extract of aspect 41, wherein exposing the host cells to the micro-current increases production of at least one metabolite compared to identical cells that are not exposed to the micro-current.

Aspect 43. The extract of aspect 42, wherein the at least one metabolite comprises a peptide or a protein.

Aspect 44. The extract of aspect 43, wherein the peptide or protein comprises casein, ovalbumin or a fragment thereof, lactalbumin, GMP, or any combination thereof.

Aspect 45. The extract of any one of aspects 27-45, wherein the host cells are exposed the micro-current for from about 30 minutes to about 72 hours.

Aspect 46. The extract of any one of aspects 25-45, further comprising an extract or a lysate from a biological device comprising a second population of host cells comprising a second DNA construct comprising the following genetic components:
(a) a gene that encodes lycopene cyclase; and
(b) a gene that encodes β-carotene hydroxylase.

Aspect 47. The extract of aspect 46, wherein the gene that encodes lycopene cyclase has SEQ ID NO. 6 or at least 70% homology thereto.

Aspect 48. The extract of aspect 46 or 47, wherein the gene that encodes β-carotene hydroxylase has SEQ ID NO. 7 or at least 70% homology thereto.

Aspect 49. The extract of any one of aspects 46-48, wherein the second DNA construct further comprises:
(c) a gene that encodes 1-deoxy-D-xylulose-5-phosphate synthase (DXS).

Aspect 50. The extract of aspect 49, wherein the gene that encodes DXS has SEQ ID NO. 8 or at least 70% homology thereto.

Aspect 51. The extract of aspect any one of aspects 46-50, wherein the second DNA construct has SEQ ID NO. 9.

Aspect 52. The extract of aspect any one of aspects 46-50, wherein the second DNA construct has SEQ ID NO. 10.

Aspect 53. The extract of aspect any one of aspects 46-52, wherein the second population of host cells comprises fungi.

Aspect 54. The extract of aspect 53, wherein the fungi comprise Saccharomyces cerevisiae.

Aspect 55. The extract of any one of aspects 25-54, further comprising an extract or lysate from a biological device comprising third population of cells comprising a third DNA construct comprising the following genetic components:
(a) a gene that encodes a hydrogenase;
(b) a gene that encodes a p-type ATPase;
(c) a gene that encodes tonB;
(d) a gene that encodes a heat shock protein; and
(e) a gene that encodes RuBisCO large subunit 1.

Aspect 56. The extract of aspect 55, wherein the gene that encodes the hydrogenase has SEQ ID NO. 11 or at least 70% homology thereto.

Aspect 57. The extract of aspect 55 or 56, wherein the gene that encodes the p-type ATPase has SEQ ID NO. 12 or at least 70% homology thereto.

Aspect 58. The extract of any one of aspects 55-57, wherein the gene that encodes the tonB has SEQ ID NO. 13 or at least 70% homology thereto.

Aspect 59. The extract of any one of aspects 55-58, wherein the gene that encodes the heat shock protein has SEQ ID NO. 14 or at least 70% homology thereto.

Aspect 60. The extract of any one of aspects 55-59, wherein the gene that encodes the RuBisCO large subunit 1 has SEQ ID NO. 15 or at least 70% homology thereto.

Aspect 61. The extract of any one of aspects 55-60, wherein the third DNA construct further comprises:
(f) a gene that encodes phosphoenol pyruvate carboxylase; and
(g) a gene that encodes phosphoenol pyruvate carboxykinase.

Aspect 62. The extract of aspect 61, wherein the gene that encodes phosphoenol pyruvate carboxylase has SEQ ID NO. 16 or at least 70% homology thereto.

Aspect 63. The extract of aspect 61 or 62, wherein the gene that encodes phosphoenol pyruvate carboxykinase has SEQ ID NO. 17 or at least 70% homology thereto.

Aspect 64. The extract of any one of aspects 55-63, wherein the third DNA construct has SEQ ID NO. 18.

Aspect 65. The extract of any one of aspects 55-63, wherein the third DNA construct has SEQ ID NO. 19.

Aspect 66. The extract of any one of aspects 55-65, wherein the third population of cells comprises fungi.

Aspect 67. The extract of aspect 66, wherein the fungi comprise Saccharomyces cerevisiae.

Aspect 68. A nutritional composition comprising the extract according to any one of aspects 1-67.

Aspect 69. The nutritional composition of aspect 68, formulated as an oral dosage form.

Aspect 70. The nutritional composition of aspect 69, wherein the oral dosage form comprises a capsule, a tablet, a sublingual strip, a buccal strip, or a beverage.

Aspect 71. The nutritional composition of any one of aspects 68-70, further comprising at least one excipient.

Aspect 72. The nutritional composition of aspect 71, wherein the excipient comprises a sweetener, a flavoring agent, a coloring agent, a stabilizer, a thickener, or any combination thereof.

Aspect 73. The nutritional composition of aspect 72, wherein the sweetener comprises saccharin, cyclamate, aspartame, steviol glycosides, or any combination thereof.

Aspect 74. The nutritional composition of aspect 72 or 73, wherein the stabilizer comprises ethanol, n-propanol, glycerol, a polyethylene glycols with a molecular weights between 200 Da and 600 Da, diethylene glycol monoethyl ether, 1,2-propylene glycol, urea, urethane, acetamide, N-methyl acetamide, N,N-diethyl acetamide, N,N-dimethyl acetamide, ethylenediamine, hydroxyethyl theophylline, tromethamine, an aliphatic amino acid, or any combination thereof.

Aspect 75. The nutritional composition of any one of aspects 72-74, wherein the thickener comprises corn starch, xanthan gum, gelatin, pectin, potato starch, tapioca starch, arrowroot, agar-agar, or any combination thereof.

Aspect 76. The nutritional composition of any one of aspects 70-75, wherein the nutritional composition is a sublingual strip or a buccal strip and wherein the at least one excipient comprises a permeation enhancer, a disintegrant, a saliva stimulating agent, a strip-forming polymer, a plasticizer, or any combination thereof.

Aspect 77. The nutritional composition of aspect 76, wherein the strip-forming polymer comprises hydroxypropyl methylcellulose (HPMC) E3, HPMC E5, HPMC E15, HPMC K-3, methylcellulose A-3, methylcellulose A-6, methylcellulose A-15, pullulan, carboxmethylcellulose or a derivative thereof, polyvinylpyrollidone (PVP) K-90, pectin, gelatin. sodium alginate, hydroxypropylcellulose, polyvinyl alcohol, maltodextrins, calcium alginate, a polyactive carbohydrate, chitosan, or any combination thereof

Aspect 78. The nutritional composition of aspect 76 or 77, wherein the permeation enhancer comprises 2,3-lauryl ether, aprotinin, azone, benzalkonium chloride, cetylpyridinium chloride, cetyltrimethyl ammonium bromide, cyclodextrin, dextran sulfate, lauric acid, lysophosphatidylcholine, menthol, phosphatidylcholine, polyoxyethylene, polysorbate 80, propylene glycol, disodium ethylenediaminetetraacetic acid (EDTA), sodium glycocholate, sodium glycodeoxycholate, sodium lauryl sulfate, sodium salicylate, sodium taurocholate, sodium taurodeoxycholate, or any combination thereof.

Aspect 79. The nutritional composition of any one of aspects 76-78, wherein the saliva stimulating agent comprises citric acid, malic acid, lactic acid, ascorbic acid, or any combination thereof.

Aspect 80. The nutritional composition of any one of aspects 76-79, wherein the disintegrant comprises cross-linked polyvinyl pyrrolidone, sodium carboxymethyl starch, sodium carboxymethyl cellulose, microcrystalline cellulose, or any combination thereof.

Aspect 81. The nutritional composition of any one of aspects 76-80, wherein the plasticizer comprises glycerol, dibutyl phthalate, polyethylene glycol, or any combination thereof.

Aspect 82. The nutritional composition of any one of aspects 68-81, further comprising one or more additional vitamins or minerals.

Aspect 83. The nutritional composition of aspect 82, wherein the one or more additional vitamins are selected from all-trans-retinols, all-trans-retinyl-esters, all-trans-β-carotene another provitamin A carotenoid, thiamine, riboflavin, niacin, niacinamide, pantothenic acid, pyridoxine, biotin, folic acid another folates, a cobalamins, ascorbic acid, a calciferol, a tocopherol, a tocotrienol, a phylloquinone, a menaquinone, a menadione, choline, nicotinamide adenine dinucleotide, nicotinamide adenine dinucleotide phosphate, or any combination thereof.

Aspect 84. The nutritional composition of aspect 82 or 83, wherein the one or more additional minerals are selected from calcium, chloride, magnesium, phosphate, potassium, sodium, chromium, copper, fluoride, iodine, iron, manganese, molybdenum, selenium, zinc, or any combination thereof.

Aspect 85. The nutritional composition of aspect 68, formulated as a powder or liquid additive to be mixed with food or a beverage.

Aspect 86. The nutritional composition of aspect 68, formulated as hydrogel microparticles.

Aspect 87. The nutritional composition of aspect 86, wherein the hydrogel microparticles comprise chitosan, acetic acid, sodium alginate, calcium alginate, a polyactive carbohydrate, glucosamine, chondroitin, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Preparation of DNA Constructs

The DNA construct was composed of the genetic components described herein and assembled in plasmid vectors (e.g., pYES2). Sequences of genes and/or proteins with desired properties were identified in GenBank; these included a gene that encodes casein, a gene that encodes an ovalbumin fragment, a gene that encodes lactalbumin, and a gene that encodes GMP. These sequences were synthesized by CloneTex Systems, Inc. (Austin, TX). Other genetic parts were also obtained for inclusion in the DNA constructs including, for example, promoter genes (e.g., GAL1 promoter), reporter genes (e.g., enhanced green fluorescent reporter protein), and terminator sequences (e.g., CYC1 terminator). These genetic parts included restriction sites for ease of insertion into plasmid vectors.

The cloning of the DNA construct into the biological devices was performed as follows. Sequences of individual genes were amplified by polymerase chain reaction using primers that incorporated restriction sites at their 5′ ends to facilitate construction of the full sequence to be inserted into the plasmid. Genes were then ligated using standard protocols to form an insert. The plasmid was then digested with restriction enzymes according to directions and using reagents provided by the enzymes' supplier (Promega). The complete insert, containing restriction sites on each end, was then ligated into the plasmid. Successful construction of the insert and ligation of the insert into the plasmid were confirmed by gel electrophoresis.

In some experiments, each gene was PCR amplified using gene-specific overlap primers and assembled sequences were sub-cloned into a pYES2 vector. PCR amplified pieces of all fragments were combined using homologous recombination technology (Gibson Assembly). Clones obtained after transformation were sequenced and analyzed for DNA sequence accuracy.

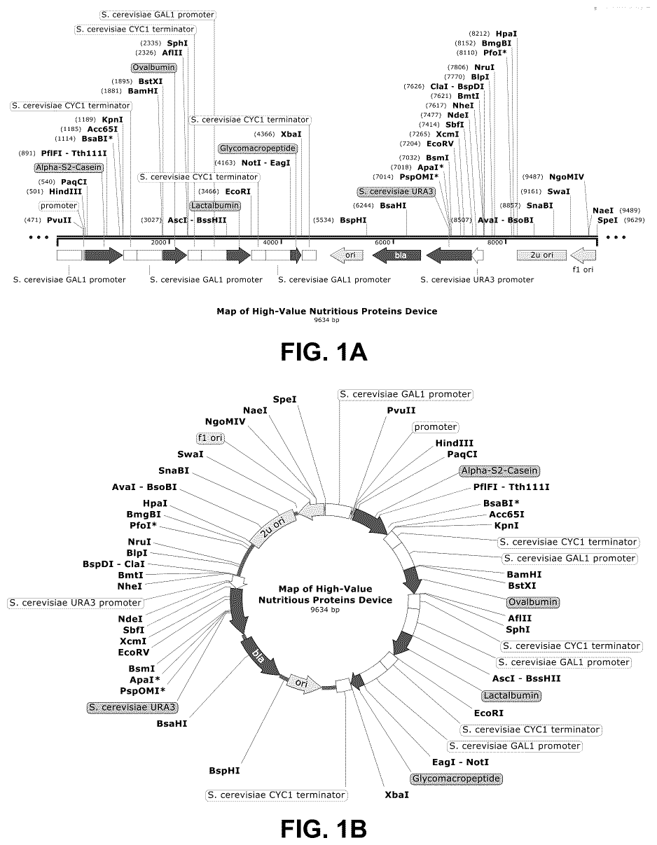

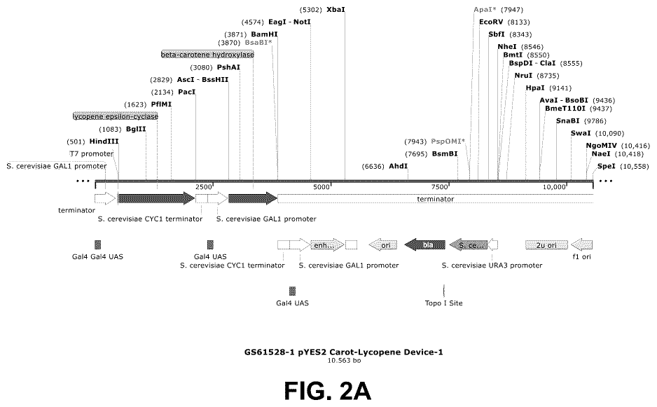

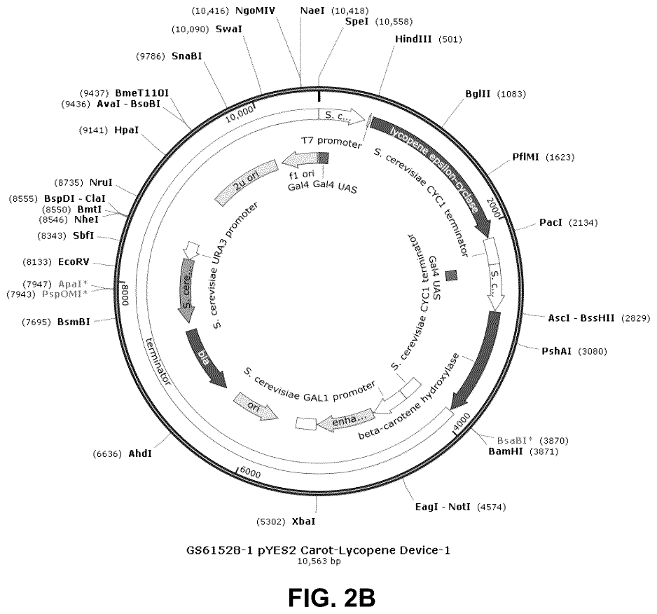

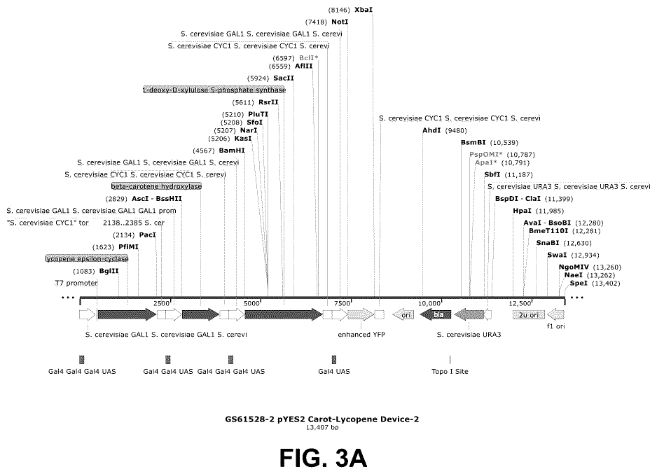

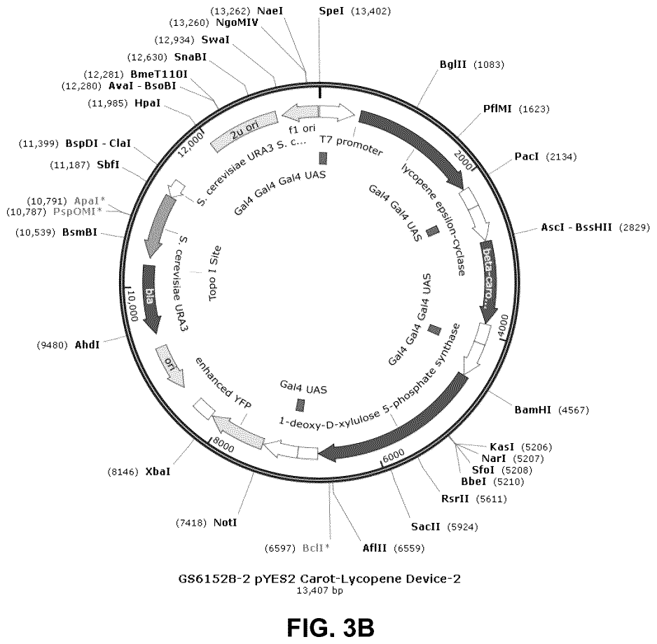

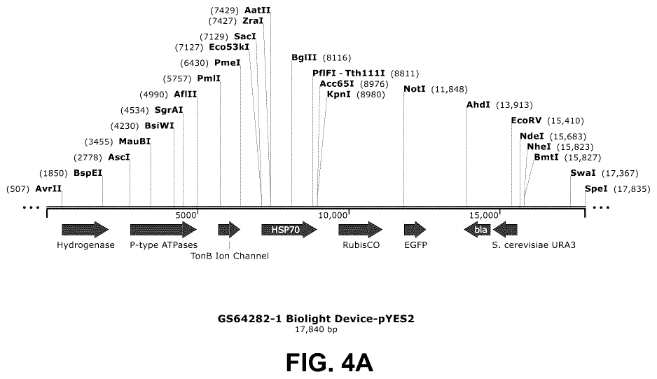

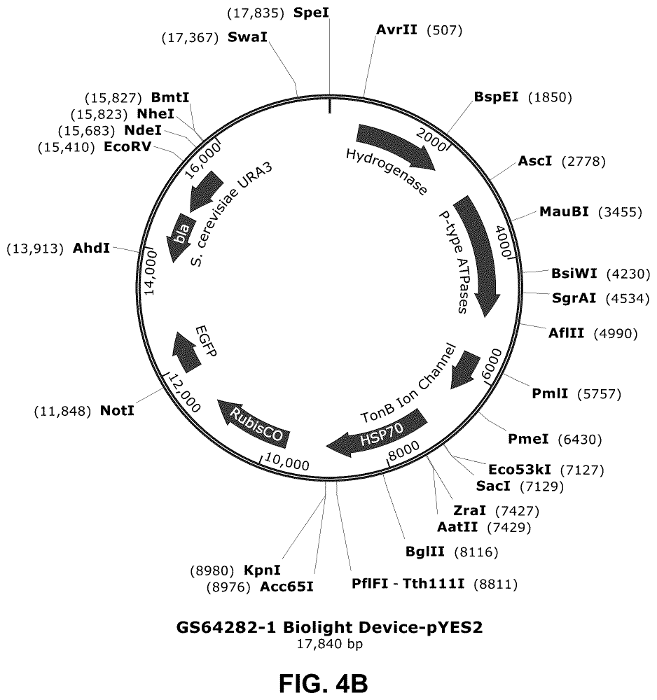

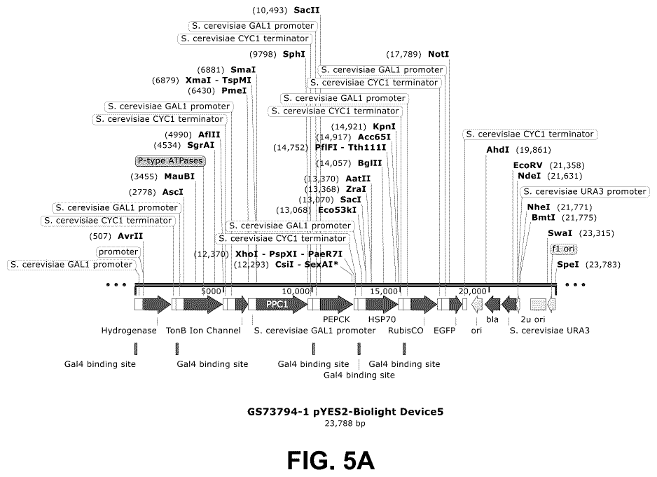

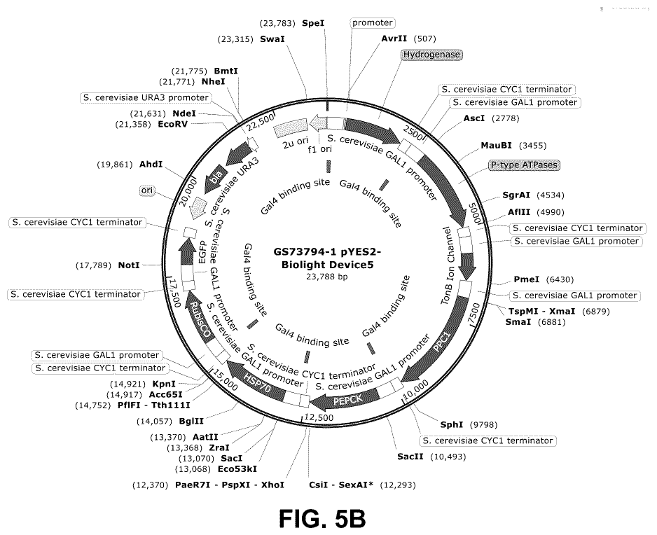

From 5′ to 3′, one version of the construct for making a complete protein producing DNA composition or extract includes (a) a gene that encodes casein, (b) a gene that encodes an ovalbumin fragment, (c) a gene that encodes lactalbumin, and (d) a gene that encodes GMP (FIGS. 1A-1B). Similar procedures were followed for the DNA constructs shown in FIGS. 2A-5B.

PCR was used to enhance DNA concentration using a Mastercycler Personal 5332 ThermoCycler (Eppendorf North America) with specific sequence primers and the standard method for amplification (Sambrook, J., E. F. Fritsch, and T. Maniatis, 1989, Molecular Cloning: A Laboratory Manual, 2nd ed., Vol. 1, Cold Spring Harbor Laboratory Press: Cold Spring Harbor, NY). Digestion and ligation were used to ensure assembly of DNA synthesized parts using restriction enzymes and reagents (PCR master mix of restriction enzymes: XhoI, KpnI, XbaI, EcoRI, BamHI, and HindIII, with alkaline phosphatase and quick ligation kit, all from Promega). DNA was quantified using a NanoVue spectrophotometer (GE Life Sciences) and a standard UV/Visible spectrophotometer using the ratio of absorbances at 260 nm and 280 nm. In order to verify final ligations, DNA was visualized and purified via electrophoresis using a Thermo EC-150 power supply.

The DNA construct was made with gene parts fundamental for expression of sequences such as, for example, native and constitutive promoters, reporter genes, and transcriptional terminators or stops. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the method described below.

### Example 2: Selection of Microorganisms and Development of Competent Cells

Some constructs were produced using transfected yeasts (Saccharomyces cerevisiae, ATCC® 200892™). Yeast cells were made competent by subjecting them to an electrochemical process adapted from Gietz and Schiestl (Nature Protocols, 2007, 2:35-37). Briefly, a single yeast colony was inoculated into 100 mL YPD (yeast extract peptone dextrose) growth media. Yeast was grown overnight on a shaker at 30° C. to OD₆₀₀=1.0. (Acceptable results were obtained with OD₆₀₀ values ranging from 0.6 to 1.8.) Cells were centrifuged at 2000 rpm in a tabletop centrifuge and resuspended in 10 mL TEL buffer (10 mM Tris-HCl, 1 mM EDTA, 0.1 M LiAc, PH=7.5) and shaken vigorously overnight at room temperature. Alternatively, INVSc1 cells were prepared to be competent using a kit from Sigma-Aldrich, Inc. Cells were again centrifuged and resuspended in 1 mL TEL buffer. Cells prepared in this manner could be stored in the refrigerator for up to one month.

### Example 3: Transformation of Microbial Cells

Competent cells were stored in the freezer until needed. Cells were thawed on ice and 100 μL of competent cells in TEL buffer were placed in a sterile 1.5 mL microcentrifuge tube. To this was added 5 μL of a 10 mg/mL solution of salmon sperm DNA (carrier DNA). Transforming DNA was added in various amounts. From 1 to 5 μg was sufficient for plasmids from commercial sources, but more DNA was required when transforming yeast with artificial DNA constructs. 10 μL of the DNA device were added to the microcentrifuge tube containing the competent yeast cells and the contents of the tube were mixed. The DNA-yeast suspension was incubated for 30 min at room temperature.

A PLATE solution (consisting of 40% PEG-3350 in 1×TEL buffer) was prepared. 0.7 mL of PLATE solution was added to the DNA-yeast suspension and the contents were mixed thoroughly and incubated for 1 h at room temperature. The mixture was placed in an electromagnetic chamber for 30 minutes. Cells were then heated at 42° C. for 5-10 minutes and 250 μL aliquots were plated on yeast malt agar to which selective growth compounds had been added. Plates were incubated overnight at 30° C.

DNA expression and effectiveness of transformation were determined by fluorescence of the transformed cells expressed in fluorescence units (FSUs) using a 20/20 Luminometer (Promega) according to a protocol provided by the manufacturer. Plasmid DNA extraction, purification, PCR, and gel electrophoresis were also used to confirm transformation. Different transformed devices were obtained. Different types of fluorescent reporter proteins were used (e.g., yellow, red, green, and cyan) for all transformed cells and/or constructs. However, the yellow fluorescent protein was preferred. When no fluorescent reporter protein was assembled, no fluorescence was observed.

S. cerevisiae cells were subjected to transformation with the modified pYES2 plasmids for producing metal- and contaminant-binding components as described above. Transformed yeast cells were incubated for 30 min at 28-30° C. Colonies of transformed yeast cells were selected, their DNA isolated and subjected to PCR amplification. Two control treatments were also carried out: (1) a negative control involving competent yeast and nuclease free water instead of a plasmid and (2) a positive control involving competent yeast with unmodified pYES2 plasmid.

Four clones were selected from a transformed plate and processed for full-length DNA sequencing. A clone with 100% DNA sequence accuracy was selected for further processing and was used to obtain a high concentration of plasmid construct at a mid-scale plasmid purification level. Yeast competent cells were transformed with the recombinant plasmid and selected on synthetic complete (SC) dropout plate deficient in uracil. Well isolated clones were isolated and preserved in YPD medium containing 15% glycerol for storage at −80° C.

### Example 4: Production of Microbial Extracts and Protein Metabolites

### Microbial Extracts Containing Protein Metabolites

The following non-limiting procedure was used to produce the disclosed extracts:

### Method

(a) Yeast transformed with the device depicted in FIGS. 1A-1B were fermented at 37° C. for 48 hours, where culture was conducted with 25 ml of device inoculum in 1 L Luria broth and having 1 μg/mL ampicillin and 100 μM isopropyl-β-D-thiogalactopyranoside (IPTG).
(b) The culture was sterilized by autoclaving at 121° C. for 30 minutes and then centrifuged.
(c) The mixture was filtered with an 0.45 μm filter to produce a supernatant composed of the desired extract.

The Protein Nutritional Device (PND), which is cloned in yeast, will be grown in the specific media with or without micro-current within a time-course process for at least a week. Then, the sample of the lysate produced by the PND will be taken and subject to protein analysis and yield under laboratory conditions. This lysate will be tested in various oral dosage forms, including sublingually and as beverage, as well as others.

Supplementary nutritional value can be added to the PND formulations including, but not limited to, vitamins A and C. This addition will make the PND more nutritional with better benefit for the consumer since these vitamins serve as antioxidants and also contribute to overall energy levels. For this purpose, the PND lysate will be mixed with a lysate from one or more additional biological devices producing other nutrients at different ratios. In some experiments, the composition also contains Stevia or an extract or lysate from a Stevia-producing device, which would improve the taste of the nutritional composition.

Exemplary biological devices to produce Stevia (steviol glycosides) are described in U.S. Pat. No. 11,365,417 and US Patent Application Publication 2023/0212588. Exemplary biological devices to produce carotenoids are described in U.S. Pat. No. 11,603,549. Genes from steviol glycoside devices and carotenoid devices can also be combined into one device to streamline processing. Additional biological devices for producing carotenoids, steviol glycosides, and organic electrolytes are described herein and shown in linear and circular forms in FIGS. 2A-5B.

Once the extracts and/or lysates are produced, they can be processed into oral dosage forms as described herein. In some aspects, lysates from the protein-producing devices and other devices described herein are mixed in any proportion (e.g. 95:1, 5:1, 2:1, 1:1, etc.) in order to produce oral dosage forms containing both a complete protein and one or more vitamins, minerals, organic electrolytes, flavorants, or the like. In some cases, lysates or extracts produced from three or more devices are mixed.

### Example 5: Hydrogel/Microparticle Encapsulation

Hydrogel beads were produced with chitosan (1% w/v) in acetic acid 3% (w/v) and sodium alginate (2% w/v). Compounds to produce the hydrogels were sodium hydroxide (NaOH) and calcium chloride (CaCl₂)), respectively. Extracts and lysates from the disclosed devices were homogenized in 1.5 mL microcentrifuge tubes. Furthermore, chitosan and sodium alginate were added to the final mixture and hydrogel beads were obtained. Proportions used to produce hydrogels with the disclosed protein-containing lysates and extracts varied. In one experiment, 900 μL of chitosan/sodium alginate solution were used with 300 μL of protein-containing lysate or extract.

### Example 6: Protein Concentration in Device Extracts

Extracts were obtained from the disclosed device cultures that were grown without or with microcurrent (100, 200, or 300 mV) in a time course experiment at 24 h, 48 h, and 72 h. 300 mV and 72 h produced the most favorable results. These samples were centrifuged for 10 minutes then the pellet was washed with cold PBS 0.1 M, pH7.4, and transferred to a 1.5 mL centrifuge tube. The washed pellets were centrifuged at 10,000×g for 2 min at 4° C. This pellet washing procedure was repeated twice, and after the last wash, pellets were ready to measure the protein content. 20 μL of each pellet were placed in a new 1.5 mL centrifuge tube, to which 400 μL of extracting solution (Colorimetric Assay kit from MSE Supplies, LLC, Tucson, AZ) were added. These samples were homogenized with an ultrasonic cell disruptor at 4° C. and centrifuged at 12,000×g for 10 min at 4° C. to remove insoluble material. The supernatant was collected and kept on ice for protein detection. Results are presented in Table 15:

TABLE 15
Protein Concentrations in Device Extracts

| | | | | Final |
|---|---|---|---|---|
| | Absorbance | Concentration | Dilution | Concentration |
| Sample | (AU) | (μg/μL) | Factor | (μg/μL) |
| Transformed Yeast, | 1.599 | 1.233 | 1.667 | 2.055 |
| 0 mV current | | | | |
| Transformed Yeast, | 1.512 | 1.141 | 1.667 | 1.902 |
| 300 mV current | | | | |
| Non-transformed | 1.084 | 0.717 | 1.667 | 1.194 |
| Yeast, 300 mV | | | | |
| current | | | | |

Transformed yeast cultures showed higher concentrations of protein, with or without microcurrent, than non-transformed yeast cultures.

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be exemplary.

What is claimed is:

1. A DNA construct comprising the following genetic components:
(a) a gene that encodes casein;
(b) a gene that encodes ovalbumin or a fragment thereof;
(c) a gene that encodes lactalbumin; and
(d) a gene that encodes glycomacropeptide (GMP).

2. The DNA construct of claim 1, wherein the gene that encodes the casein has SEQ ID NO. 4 or at least 70% homology thereto.

3. The DNA construct of claim 1, wherein the gene that encodes the fragment of ovalbumin has SEQ ID NO. 1 or at least 70% homology thereto.

4. The DNA construct of claim 1, wherein the gene that encodes lactalbumin has SEQ ID NO. 3 or at least 70% homology thereto.

5. The DNA construct of claim 1, wherein the gene that encodes GMP has SEQ ID NO. 2 or at least 70% homology thereto.

6. The DNA construct of claim 1, wherein the construct comprises from 5′ to 3′ the following genetic components in the following order: (a) the gene that encodes casein; (b) the gene that encodes ovalbumin or a fragment thereof; (c) the gene that encodes lactalbumin; and (d) the gene that encodes GMP.

7. The DNA construct of claim 1, wherein the construct comprises from 5′ to 3′ the following genetic components in the following order: (a) a gene that encodes a casein having SEQ ID NO. 4 or at least 70% homology thereto; (b) a gene that encodes a fragment of ovalbumin having SEQ ID NO. 1 or at least 70% homology thereto; (c) a gene that encodes lactalbumin having SEQ ID NO. 3 or at least 70% homology thereto; and (d) a gene that encodes GMP having SEQ ID NO. 2 or at least 70% homology thereto.

8. The DNA construct of claim 7, wherein the construct comprises from 5′ to 3′ the following genetic components in the following order: (a) the gene that encodes casein having SEQ ID NO. 4 or at least 70% homology thereto, (b) a CYC1 terminator, (c) a GAL1 promoter, (d) the gene that encodes a fragment of ovalbumin having SEQ ID NO. 1 or at least 70% homology thereto, (e) a CYC1 terminator, (f) a GAL1 promoter, (g) the gene that encodes lactalbumin having SEQ ID NO. 3 or at least 70% homology thereto, (h) a CYC1 terminator, (i) a GAL1 promoter, and (j) the gene that encodes GMP having SEQ ID NO. 2 or at least 70% homology thereto.

9. The DNA construct of claim 1, wherein the DNA construct has SEQ ID NO. 5.

10. A vector comprising the DNA construct of claim 1.

11. The vector of claim 10, wherein the vector is a plasmid selected from pWLneo, pSV2cat, pOG44, pXT1, pSG, pSVK3, pBSK, pBSKII, pYES, pYES2, pET, pBAD, pUC, or pUC19.

12. A biological device comprising host cells transformed with the DNA construct of claim 1.

13. The biological device of claim 12, wherein the host cells comprise Saccharomyces cerevisiae.

14. An extract produced by culturing the biological device of claim 13 in a culture medium, wherein the extract comprises casein, ovalbumin or a fragment thereof, lactalbumin, and GMP.

15. The extract of claim 14, wherein the cells are exposed to a micro-current of from about 50 mV to about 300 mV during the culturing of the cells.

16. The extract of claim 15, wherein exposing the host cells to the micro-current increases production of at least one metabolite compared to identical cells that are not exposed to the micro-current.

17. The extract of claim 16, wherein the at least one metabolite comprises casein, ovalbumin or a fragment thereof, lactalbumin, GMP, or any combination thereof.

18. A nutritional composition comprising the extract of claim 14.

19. The nutritional composition of claim 18, formulated as an oral dosage form comprising a capsule, a tablet, a sublingual strip, a buccal strip, or a beverage.

20. The nutritional composition of claim 18, further comprising one or more additional vitamins or minerals.
